(19) United States
(12) Patent Application Publication
(10) Pub. No.: US 2026/0132358 A1
(43) Pub. Date: May 14, 2026

(54) Systems, Methods And Compositions For The Preservation And Rehabilitation Of Living Allogenic Heart Valves

(71) Applicant: THE TRUSTEES OF COLUMBIA UNIVERSITY IN THE CITY OF NEW YORK, New York, NY (US)

(72) Inventors: David M. Kalfa, Miami, FL (US); Elizabeth M. Cordoves, New York, NY (US); Gordana Vunjak-Novakovic, New York, NY (US)

(21) Appl. No.: 19/377,829

(22) Filed: Nov. 3, 2025

Related U.S. Application Data
(63) Continuation-in-part of application No. PCT/US2024/027291, filed on May 1, 2024.
(60) Provisional application No. 63/499,394, filed on May 1, 2023.
(60) Provisional application No. 63/591,671, filed on Oct. 19, 2023.
(60) Provisional application No. 63/898,027, filed on Oct. 13, 2025.

(51) Int. Cl. C12M 3/00; A01N 1/126; C12M 1/00; C12M 1/12; C12M 1/34; C12M 3/06

(52) U.S. Cl. CPC C12M 21/08; A01N 1/126; C12M 25/10; C12M 27/14; C12M 29/12; C12M 41/00; C12M 41/40

(57) ABSTRACT

A tissue culture system, comprising: a support configured to be rotated about an axis of rotation; and one or more culture vessels configured to be mounted to at least a portion of the support, a culture vessel comprising: (i) a chamber configured to receive a biological tissue, and (ii) a conduit in communication with the chamber and configured to facilitate a flow of a composition, wherein the support is configured to cause rotation of the one or more culture vessels during rotation of the support, and wherein the conduit is configured to allow for flow of the composition through the chamber during rotation of the culture vessel. Related methods and tissue treatment compositions are also provided.

### CROSS REFERENCE TO RELATED APPLICATIONS

The present application claims priority to and the benefit of U.S. Provisional Patent Application No. 63/898,027 (filed Oct. 13, 2025). The present application is also a continuation-in-part of international patent application PCT/US2024/027291 (filed May 1, 2024), which application claims priority to and the benefit of U.S. Provisional Patent Application No. 63/591,671 (filed Oct. 19, 2023) and U.S. Provisional Patent Application No. 63/499,394 (filed May 1, 2023). All foregoing applications are incorporated by reference herein in their entireties for any and all purposes.

### GOVERNMENT RIGHTS

This invention was made with government support under EB027062 awarded by the National Institutes of Health. The government has certain rights in the invention.

### TECHNICAL FIELD

The present disclosure generally relates to the preservation and rehabilitation of biological tissues, and more particularly to systems and compositions for treating living allogenic heart valves.

### BACKGROUND

The current treatment for valvular disease includes the use of mechanical valves and tissue-based valves. While mechanical valves are relatively durable, such valves cannot grow within the body and can be thrombogenic. Tissue-based valves are able to mimic native valve hemodynamics. However, such valves are also unable to grow and are subject to structural degradation. Accordingly, there is a long-felt need in the art for systems and methods for preserving biological tissues, in particular tissue-based valves.

### SUMMARY

In meeting the described long-felt needs, the present disclosure provides a tissue culture system, comprising: a support configured to be rotated about an axis of rotation; and one or more culture vessels configured to be mounted to at least a portion of the support, a culture vessel comprising: (i) a chamber configured to receive a biological tissue, and (ii) a conduit in communication with the chamber and configured to facilitate a flow of a composition, wherein the support is configured to cause rotation of the one or more culture vessels during rotation of the support, and wherein the conduit is configured to allow for flow of the composition through the chamber during rotation of the culture vessel.

Also provided is a culture vessel configured for culturing a biological tissue, the culture vessel comprising: a chamber configured to receive a biological tissue; and a conduit in communication with the chamber and configured to facilitate a circumferential flow of a composition, wherein at least a portion of the chamber and the conduit are configured to be mounted to a support, and wherein the chamber and the conduit are configured to be rotated about an axis of rotation so as to cause the composition to flow through the chamber.

Further provided is a method for culturing biological tissue, the method comprising: rotating an annular culture vessel having a biological tissue retained therein so as to give rise to motion of a fluid composition within the annular culture vessel, the rotating giving rise to flow of the fluid composition along the biological tissue.

Additionally disclosed is a composition for treating a biological tissue, comprising: a base solvent: at least one of an animal serum and an animal-free serum: an antioxidant: insulin, an insulin analog, or an insulin mimetic: a glutamine: at least one of an antibiotic and an antimycotic; and optionally albumin and ascorbic acid.

Further provided is a system configured for culturing a biological tissue is provided, the system includes a support extending between a first end and a second end, at least a portion of the support being configured to be rotated about an axis of rotation: one or more culture vessel configured to be mounted to at least a portion of the support, the culture vessel including a chamber being configured to receive a biological tissue and a conduit in communication with the chamber being configured to facilitate a flow of a composition: wherein the support is configured to cause rotation of the culture vessel during rotation of the support and the conduit is configured to allow for flow of the composition through the chamber during rotation of the culture vessel.

In some embodiments, at least a portion of the support is substantially tubular. In some embodiments, at least a portion of the culture vessel is substantially annular. In some embodiments, the culture vessel is configured to rotate about the axis of rotation of the support during rotation of the support. In some embodiments, at least a portion of the culture vessel is configured to be mounted to the support circumferentially.

In some embodiments, the conduit of the culture vessel extends between a first end and a second end, and each of the first end of the conduit and the second end of the conduit is secured to the chamber of the culture vessel. In some embodiments, the conduit of the culture vessel defines a first opening at the first end of the conduit and a second opening at the second end of the conduit, and each of the first opening and the second opening are in communication with the chamber of the culture vessel. In some embodiments, one or more of the first end of the conduit of the culture vessel and the second end of the conduit of the culture vessel includes a first coupling member, the chamber of the culture vessel includes a second coupling member, and the first coupling member of the conduit is configured to be releasably coupled to the second coupling member of the chamber.

In some embodiments, the chamber of the culture vessel includes a platform, and the platform is configured to secure at least a portion of the biological tissue within the chamber.

In some embodiments, one or more of the chamber and the conduit of the culture vessel is configured to facilitate unidirectional flow of the composition through the chamber.

In some embodiments, the biological tissue is an allograft. In some embodiments, the allograft is a valve of a heart of an animal.

Also disclosed is a culture vessel configured for culturing a biological tissue is provided, the culture vessel includes a chamber configured to receive a biological tissue: a conduit in communication with the chamber configured to facilitate a flow of a composition: wherein at least a portion of the chamber and the conduit are configured to be mounted to a support and the chamber and the conduit are configured to be rotated about an axis of rotation to cause the composition to flow through the chamber.

In some embodiments, at least a portion of the culture vessel is substantially annular. In some embodiments, the conduit of the culture vessel extends between a first end and a second end and each of the first end of the conduit, and the second end of the conduit is secured to the chamber of the culture vessel. In some embodiments, the conduit of the culture vessel defines a first opening at the first end of the conduit and a second opening at the second end of the conduit, and each of the first opening and the second opening are in communication with the chamber of the culture vessel.

In some embodiments, one or more of the chamber and the conduit of the culture vessel is configured to facilitate unidirectional flow of the composition through the chamber.

A method for culturing biological tissue is provided, the method includes collecting a biological tissue: treating the biological tissue with a composition: storing the biological tissue in the composition in a chamber of a culture vessel: mounting the culture vessel to at least a portion of a support; and rotating at least a portion of the support about an axis of rotation.

In some embodiments, mounting the culture vessel includes inserting at least a portion of the support through at least a portion of the culture vessel. In some embodiments, rotating the support includes rotating the culture vessel about the axis of rotation. In some embodiments, rotating the culture vessel about the axis of rotation includes causing the composition to flow through at least a portion of the chamber of the culture vessel. In some embodiments, storing the biological tissue includes securing at least a portion of the biological tissue to a platform within the chamber of the culture vessel.

A composition for treating a biological tissue is provided, the composition includes a base solvent: one or more supplement selected from a group comprising: glucose, 1-glucose, dextran, phenol red, sodium pyruvate, HEPES, animal serum, and an animal serum-free formulation configured for cell culture; and one or more antibiotic selected from a group comprising: penicillin, streptomycin, vancomycin, imipenem, amphotericin, gentamicin, cefotaxime, fluconazole, polymyxin, and lincomycin.

In some embodiments, the one or more supplement further comprises one or more of a signaling molecule and a signaling compound. In some embodiments, the one or more supplement further comprises one or more of a cell-derived molecule and a cell-derived compound, one or more of an immunomodulatory molecule and an immunomodulatory compound, one or more of an antiapoptotic molecule and an antiapoptotic compound, and one or more of a metabolic acid molecule and a metabolic acid compound.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various aspects discussed in the present document. In the drawings:

FIG. 1 provides exemplary depictions of a living allogenic heart valve replacement.

FIG. 2 depicts data showing lifetime risk of bioprosthetic valves (BP) which are associated with high reoperation rates.

FIG. 3 depicts statistical data indicating patients having valve replacement surgery experience decreasing freedom for homograft failure in subsequent years from surgery.

FIG. 4 depicts statistical data showing the decrease in the proportion of surviving pediatric patients with bovine conduits. Small conduits include those with a diameter of 12-14 mm, medium conduits include those with a diameter of 16-18 mm, and large conduits include those with a 20-22 mm diameter.

FIG. 5 depicts statistical data showing a declining freedom from pulmonary valve dysfunction for cryopreserved homografts.

FIG. 6A and FIG. 6B depict photomicrographs of Hematoxylin and eosin (H&E) stained cusps from valves from orthotopic heart transplants. FIGS. 6A and 6B both show the expected layered architecture, as well as a normal complement of interstitial and endothelial cells. Images are 90× magnifications.

FIGS. 7A-7B. FIG. 7A illustrates an H&E stained un-implanted cryopreserved valve tissue, and FIG. 7B illustrates and H&E stained cryopreserved explant removed at autopsy 2 days after implantation. FIGS. 7A-7B show reduced interstitial cellularity. Original magnification 1753; insets 903.

FIG. 8 depicts a schematic of an exemplary concept as contemplated herein, in which a valvular homograft is collected from a donor, and the homograft is prepared for implantation.

FIG. 9 and FIG. 10 show statistical data demonstrating that “fresh” homografts result freedom from structural deterioration matching or surpassing that of cryopreserved homografts.

FIG. 11 is a graph of statistical data showing the incidence of aortic valve regurgitation as a function of valve storage time.

FIGS. 12A, 12B and 12C depict the histologic appearance of fresh valves prior to preservation. FIG. 12A depicts the cellularity of the leaflet within the elastic layer.

FIG. 12B depicts the integrity of the collagen bands. FIG. 12C depicts that the endothelial layer is intact.

FIGS. 13A, 13B and 13C depict histology slides illustrating the histologic appearance of a heart valve after 4 weeks of preservation. FIG. 13A highlights the heart valve leaflet cellularity within the elastic layer, FIG. 13B highlights the heart valve leaflet integrity of the collagen bands, and FIG. 13C highlights the intact endothelial layer.

FIGS. 14A, 14B and 14C illustrate the histologic appearance of a valve after 3 months of preservation. FIG. 14A highlights the heart valve leaflet cellularity within the elastic layer, FIG. 14B highlights the heart valve leaflet integrity of the collagen bands, and FIG. 14C highlights the endothelial layer.

FIG. 15 is a schematic illustrating an allograft appropriate for the recipient being selected from long term storage, and then implanted.

FIG. 16 is a Venn diagram depicts several environmental elements considered in developing the techniques for preserving a valvular allograft's viability and capacity for growth.

FIG. 17 depicts and exemplary procedure for harvesting a valvular graft, sterilization of the graft and rehabilitation of the valve in a solution laden with physiologic biochemical cues.

FIG. 18 illustrates the systolic mechanical forces on the valve tissue.

FIG. 19 illustrates the diastolic mechanical forces on the valve tissue.

FIG. 20 shows an exemplary embodiment of a system as contemplated by the present disclosure showing a number of annular culture vessels mounted in parallel onto a rotating tubular support or mandrel.

FIG. 21 depicts an exemplary annular culture vessels including a chamber for receiving a heart valve tissue connected to a looped conduit connected to either end of the tissue chamber.

FIG. 22 depicts an exemplary culture vessel in accordance with an exemplary embodiment.

FIG. 23 is an enlarged view of chamber in accordance with an exemplary embodiment.

FIG. 24 depicts additional features of the bioreactor loop including a viewing window, backflow channels, and inlet.

FIG. 25 depicts an exemplary protocol for intake and processing of porcine right ventricular outlet (RVOT) tissue.

FIG. 26 depicts the contents of a first preservation solution, referred to herein as PS1.

FIG. 27 depicts the contents of a second preservation solution, referred to herein as PS2.

FIG. 28, FIG. 29 and FIG. 30 depict an exemplary mechanical rocker used for applying mechanical stimulation to tissues in accordance with exemplary embodiments.

FIG. 31 illustrates the results of whole leaflet viability testing after one week of storage. Samples were tested with PS1 and PS2. Some samples included mechanical stimulation (mech) and ATP.

FIG. 32 depicts viability results of whole leaflet viability testing after two weeks of storage. Testing showed that PS1 and PS2 were able to maintain valve leaflet viability up to 2 weeks in storage.

FIG. 33 illustrates viability results of whole leaflet viability testing after three weeks of storage.

FIG. 34 depicts the results of pulmonary artery (PA) viability testing after one week of storage.

FIG. 35 depicts the results of PA viability testing after two weeks of storage.

FIG. 36 depicts the results of PA viability testing after three weeks of storage.

FIG. 37 depicts an exemplary rationale and method for monitoring tissue glucose uptake between media changes for providing insight as to the metabolic activity of stored valvular tissue.

FIG. 38 and FIG. 39 depict culture media glucose levels determined for fridge-stored valve leaflets using PS1 (FIG. 38) and PS2 (FIG. 39). N>1, Samples within each group come from different porcine donors (between 1-4 technical replicates per sample). Statistical analysis: one-way ANOVA with each group compared to the cell-free control.

FIG. 40 and FIG. 41 depict culture media glucose levels for fridge-stored valve leaflets using PS1 (FIG. 40) and PS2 (FIG. 41) with mechanical stimulation. N>1, Samples within each group come from different porcine donors (between 1-4 technical replicates per sample). Statistical analysis: one-way ANOVA with each group compared to the cell-free control.

FIG. 42 and FIG. 43 depict culture media glucose levels for incubator-stored valve leaflets using PS1 (FIG. 42) and PS2 (FIG. 43) with mechanical stimulation. N>1

Samples within each group come from different porcine donors (between 1-4 technical replicates per sample). Statistical analysis: one-way ANOVA with each group compared to the cell-free control.

FIG. 44, FIG. 45 and FIG. 46 show porcine leaflet architecture after one-week storage for PS1 at 37° C. (FIG. 44), PS1 with ATP at 37° C. (FIG. 45), and PS1 with mechanical stimulation and ATP at 37° C. (FIG. 46). The scalebar in each of FIGS. 44-46 is 200 microns.

FIGS. 47-54 illustrate fluorescence imaging results of porcine pulmonary valve leaflets after one week of storage. Hoechst (all cells), Propidium Iodide (dead cells) and Calcein (live cells) dyes were to show the presence of live and dead cells. Control samples included fresh tissue (FIG. 47) and EtOH killed control tissue (FIG. 48). Imaging was performed on samples with PS1 with mechanical stimulation and 37° C. (incubator) storage (FIG. 49), PS1 and 4° C. (refrigerator) storage (FIG. 50) and PS1 with mechanical stimulation and 4° C. (refrigerator) storage (FIG. 51). Imaging was performed on samples with PS2 with mechanical stimulation and 37° C. (incubator) storage (FIG. 52), PS2 and 4° C. (refrigerator) storage (FIG. 53) and PS2 with mechanical stimulation and 4° C. (refrigerator) storage (FIG. 54). All preservation conditions show presence of viable cells on the leaflet surface after 1 week of storage.

FIGS. 55-62 illustrate imaging tests (Hoechst, Propidium Iodide and Calcein) to show the presence of live and dead cells in porcine pulmonary valve leaflets after two weeks of storage. Control samples included fresh tissue (FIG. 55) and EtOH killed control tissue (FIG. 56). Imaging was performed PS1 with mechanical stimulation and 37° C. (incubator) storage (FIG. 57), PS1 and 4° C. (refrigerator) storage (FIG. 58) and PS1 with mechanical stimulation and 4° C. (refrigerator) storage (FIG. 59). Imaging was performed PS2 with mechanical stimulation and 37° C. (incubator) storage (FIG. 60), PS2 and 4° C. (refrigerator) storage (FIG. 61) and PS2 with mechanical stimulation and 4° C. (refrigerator) storage (FIG. 62). Live/dead imaging of the leaflet surface demonstrated that mechanical stimulation at 4° C. is associated with increased cell death.

FIG. 63 shows survival results for pediatric patients showing freedom from pulmonary valve dysfunction in recipients of bovine conduits (left graph) and cryopreserved homograft (right panel).

FIG. 64 provides a schematic illustrating the general concept behind homograft partial heart transplants.

FIG. 65 provides illustrative indications of “fresh” homograft performance.

FIG. 66 provides exemplary depictions of a living allogenic heart valve replacement.

FIG. 67 provides considerations related to preservation of valve allograft tissue physiology.

FIG. 68 provides illustrative information concerning porcine pulmonary allograft collection and viability testing.

FIG. 69 provides an exemplary TUNEL analysis of valve leaflet viability.

All scalebars are 100 μm. Bars are mean+/−SD, one-way ANOVA was performed, each group is compared to fresh tissues. Three regions of interest were evaluated per datapoint.

FIG. 70 provides an exemplary phenotypic analysis of resident valve leaflet cells. Expression levels were obtained using RT qPCR. N=2-9, 2 technical replicates per datapoint. Mean+/−SD, one-way ANOVA, each group was compared to fresh tissues.

FIG. 71 provides exemplary histologic assessments of valve leaflet architecture preservation.

FIG. 72 provides background information concerning creation of a biomimetic mechanical environment for long-term storage.

FIG. 73 provides a depiction of an exemplary bioreactor according to the present disclosure.

FIG. 74 provides an illustration of an embodiment of a bioreactor according to the present disclosure.

FIG. 75 provides information concerning considerations related to intrinsic immunogenicity of living valvular tissue.

FIG. 76 provides aortic leaflet histological sections demonstrating significant microarchitectural shifts over the course of weeks-long storage in electrolyte solution (commercially-available Hanks Balanced Salt Solution).

FIG. 77 depicts the constituent components of the Allogenic Valve Solution (AVS).

FIG. 78 depicts an exemplary automated motor for generating bioreactor rotation.

FIG. 79 is photograph of an exemplary automated motor for inducing bioreactor rotation. The image on the left is a view looking through the observation window to visualize a valve mounted in the chamber. The bioreactor mounted on the automated motor is capable of inducing valve/open close behavior.

FIG. 80 provides an exemplary prototype of an embodiment of the bioreactor using a pulley system to cause rotation.

FIG. 81 provides an exemplary prototype of an embodiment of the bioreactor using a pulley system and an automated motor to cause rotation.

FIG. 82 depicts images from an ANSYS® FLUENT® model of the bioreactor system. ANSYS® FLUENT® was employed to model pressure and flow velocity magnitude inside the moving bioreactor.

FIG. 83 provides an exemplary velocity magnitude heatmap and a pressure magnitude heatmap of the flow in the bioreactor determined using ANSYS® FLUENT® software. Heatmaps of velocity and pressure magnitudes were developed along the coronal cross-section of the bioreactor.

FIG. 84 provides an exemplary velocity magnitude heatmap and a pressure magnitude heatmap of the flow in the bioreactor showing that physiologic flow velocities were achieved in computer-simulated models of bioreactor rotation determined using ANSYS® FLUENT® software.

FIG. 85 is a schematic showing how each bioreactor loop containing tissue is mounted onto a rotating mandrel embodiment multiple loops mounted on a rotating mandrel.

FIG. 86 is an exemplary experimental schematic for interrogating the role of temperature in ex vivo valve viability.

FIG. 87 shows results from experiments evaluating valve leaflet tissue and pulmonary artery tissue viability when stored under static condition in HBSS or AVS in hypothermic conditions (4° C.) for up to 3 weeks. Bars represent means+/−SD, two-way ANOVA, all columns were compared to fresh tissue controls.

FIG. 88 shows results from experiments evaluating valve leaflet tissue and pulmonary artery tissue viability when stored in HBSS or AVS in normothermic conditions (37° C.) for up to 3 weeks. Bars represent means+/−SD, two-way ANOVA, all columns were compared to fresh tissue controls.

FIG. 89 is an exemplary experimental overview for interrogating the role of biochemical cues in ex vivo valve viability. Exemplary metabolic cues and antioxidants as well as phenotypic cues were evaluated.

FIG. 90 shows results from valve leaflet tissue viability experiments conducted over a 7-week time course with AVS alone or supplemented with biochemical cues under normothermic conditions. Results are shown as means+/−SD, 2-3 technical replicates were collected per datapoint, and two-way ANOVA was performed. All columns were compared to fresh tissue controls.

FIG. 91 shows results from pulmonary artery tissue viability experiments conducted over a 7-week time course with AVS alone or supplemented with biochemical cues under normothermic conditions. Results are shown as means+/−SD, 2-3 technical replicates were collected per datapoint, and two-way ANOVA was performed. All columns were compared to fresh tissue controls.

FIG. 92 is a graph showing cumulative tissue glucose consumption. Media glucose testing shows a linear increase in the cumulative tissue glucose consumption. Samples have an N of 2 to 20: each plotted point shown represents a mean value.

FIG. 93 shows an exemplary experimental model for evaluating whether it is possible to recover viable cells from stored valve tissues. Brightfield images of cells isolated form valve samples stored in AVS for 4 weeks.

FIG. 94 shows brightfield images of cells 24 hours after being isolated from valve tissues that had been stored in AVS with or without biochemical cues for 7 weeks in normothermic conditions.

FIG. 95 shows results from a cell proliferation assay conducted on valve interstitial cells 36 hours after being isolated from valves that were stored for 7 weeks. Results indicated that the isolated cells demonstrate proliferation capacity.

FIG. 96 shows histological analysis of H&E staining of pulmonary valve leaflets that were stored or preserved under normothermic conditions in AVS with or without biochemical cues for 5 weeks or 6 weeks. A fresh tissue sample was also stained for comparison.

FIG. 97: Graphical Abstract. Created using BioRender.com.

FIGS. 98A-98G: Valvular tissue viability in hypothermic versus normothermic storage. FIG. 98A. Porcine sample procurement. Valvular leaflet (FIG. 98B) and pulmonary artery (FIG. 98C) tissues show a rapid decrease in viability in static cold storage in Valve Preservation Solution. Leaflet (FIG. 98D) and pulmonary artery (FIG. 98E) tissues similarly show a loss of viability by 2 weeks ex vivo in Hanks Balanced Salt Solution with Knock Out Serum. (FIG. 98F) Whole tissue viability in normothermic HBSS with Knock Out Serum is significantly reduced in the first 3 weeks ex vivo, versus normothermic VPS where viability is maintained (FIG. 98G). Mean+/−SD, 2-3 technical replicates per datapoint with N=2-20 biological replicates per group and timepoint. Statistics were Kruskal-Wallis test with Dunn's multiple comparisons test comparing longitudinal values to baseline. Fresh valve samples are the same in FIGS. 98B/D and FIGS. 98C/E (also the same as fresh valves shown in FIGS. 3 and 4). VPS=Valve Preservation Solution. HBSS=Hanks Balanced Salt Solution.

FIGS. 99A-99F: Valvular tissue viability in normothermic storage. Valves demonstrate preserved leaflet (FIG. 99A) and pulmonary artery (FIG. 99B) viability for up to 7 weeks ex vivo, with a minimal decrease in lactate release over time (FIG. 99C). FIG. 99D. Supernatant lactate dehydrogenase decreases significantly throughout the first weeks of ex vivo storage. FIG. 99E. Allografts show consistent glucose consumption over time. FIG. 99F. Brightfield imaging of valve interstitial cells isolated from 4-week stored valves. For FIG. 99A-FIG. 99B, 2-3 technical replicates were obtained for each datapoint. For FIG. 99A-FIG. 99E, mean+/−SD are shown, with N=2-20 individual tissue samples per group and timepoint. Statistics were Kruskal-Wallis test with Dunn's multiple comparisons test comparing each longitudinal timepoint to the first-available value. Fresh samples are the same as those shown in FIGS. 2 and 4.

FIGS. 100A-100I: Parameter-controlled study of biochemical additives.

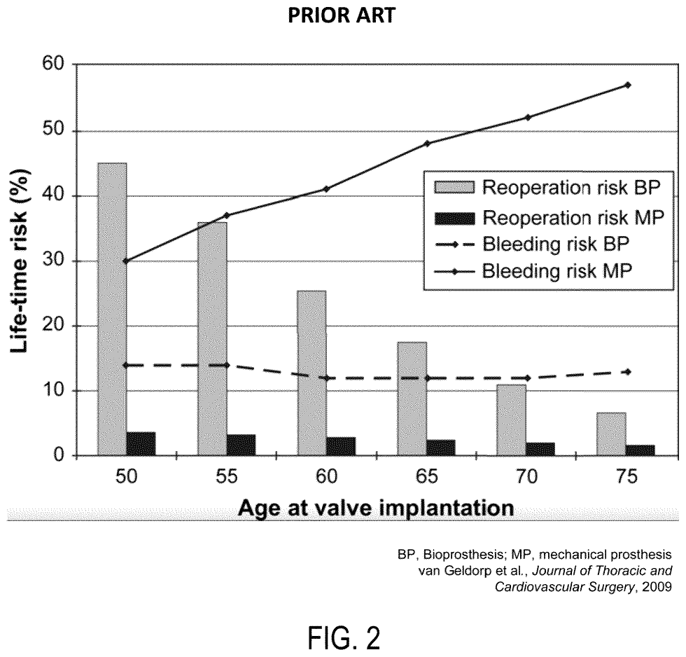

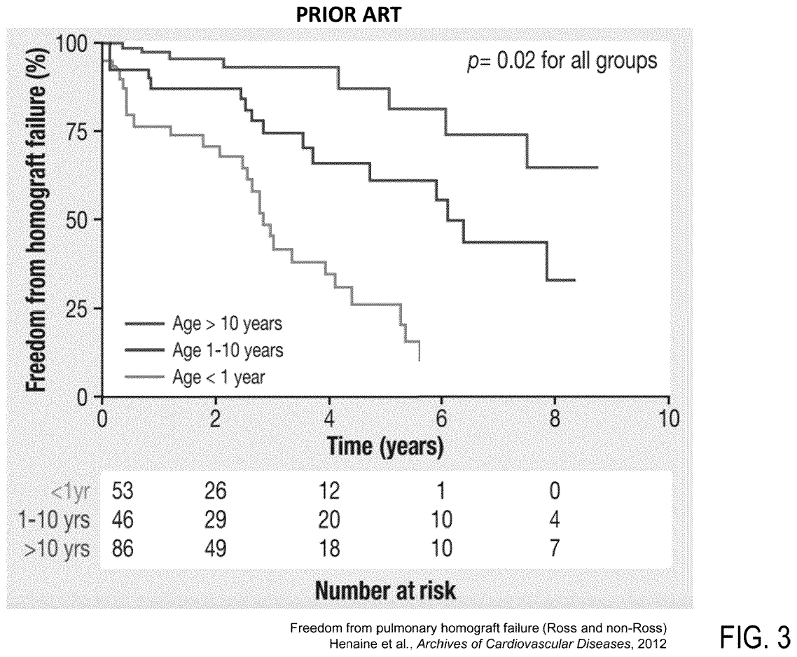

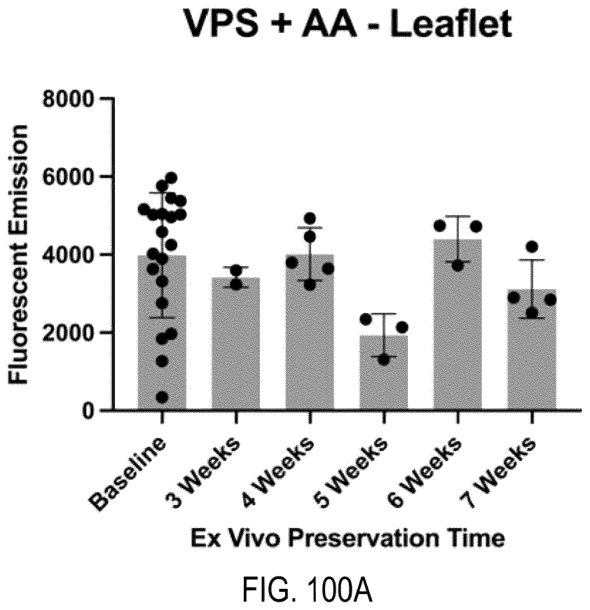

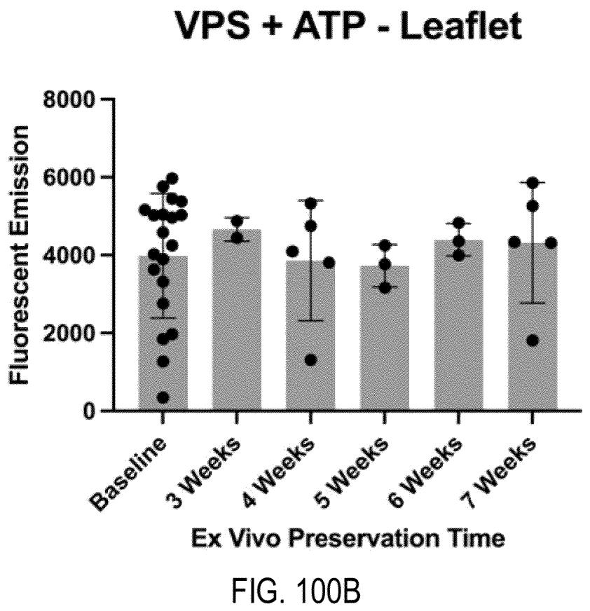

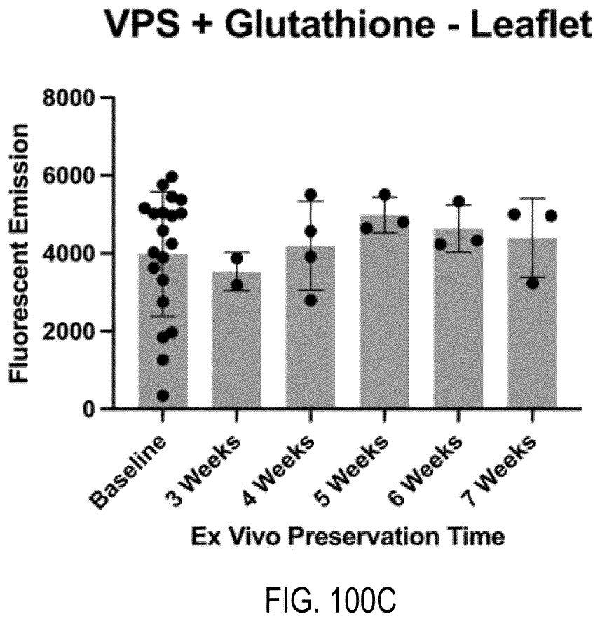

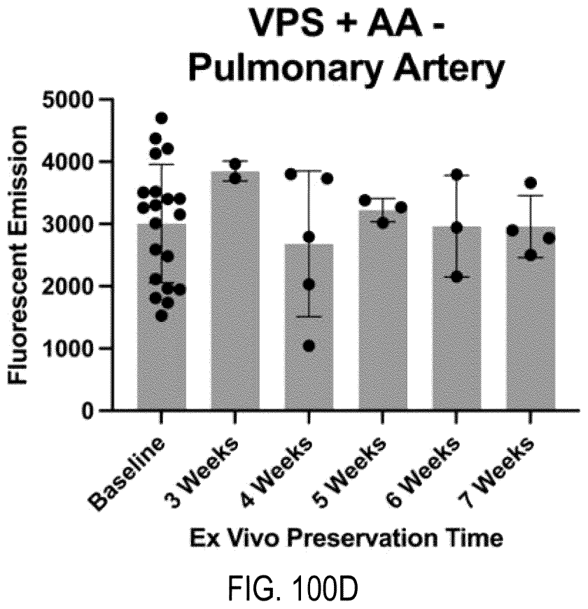

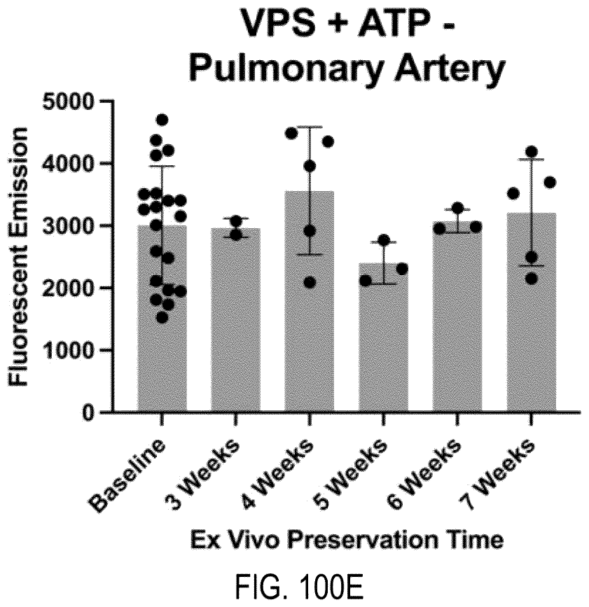

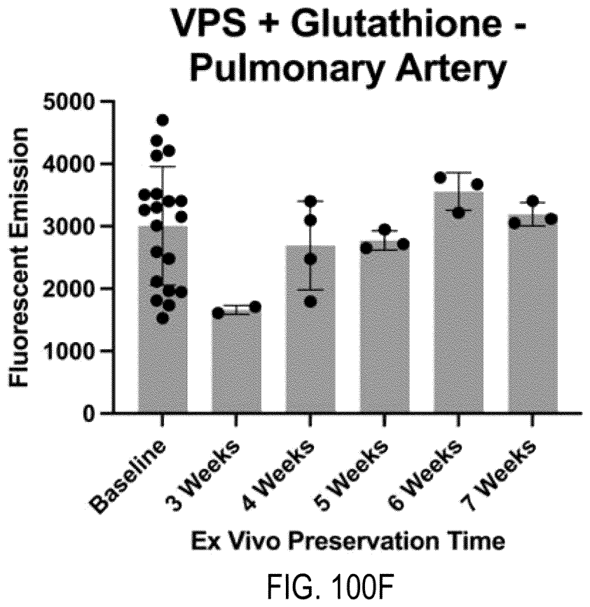

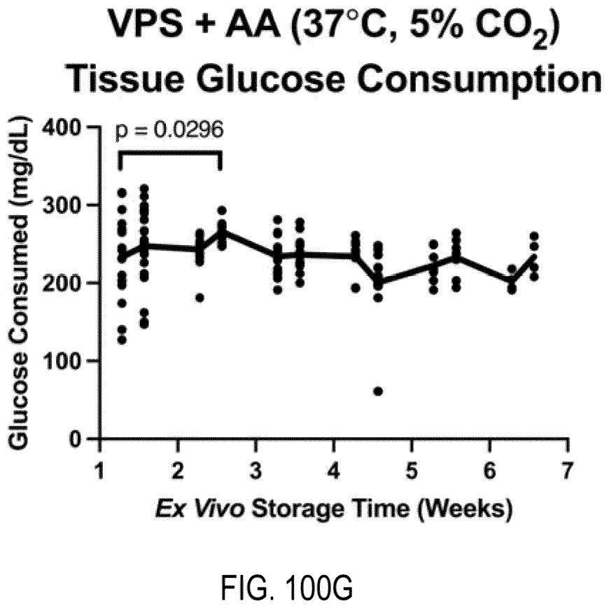

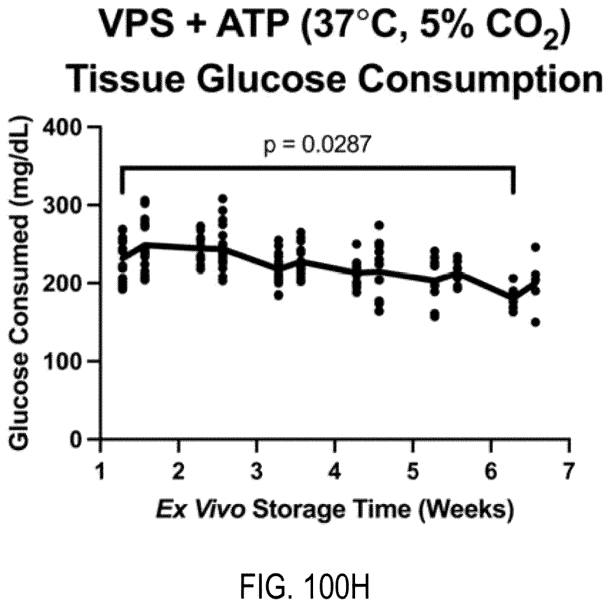

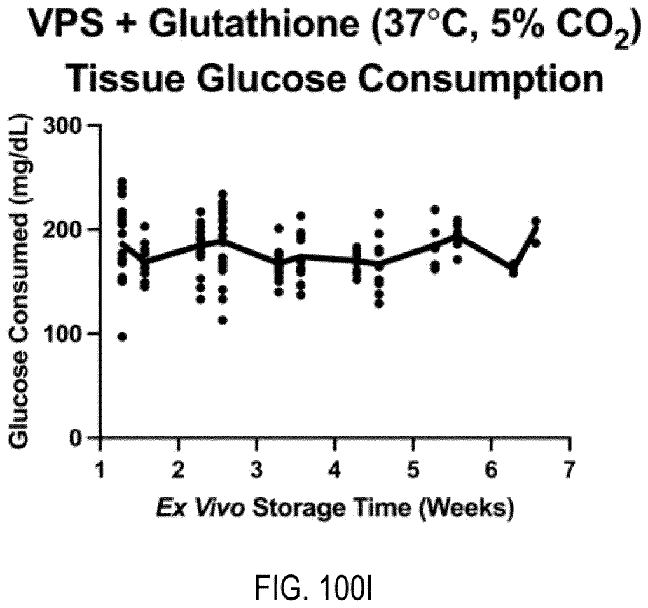

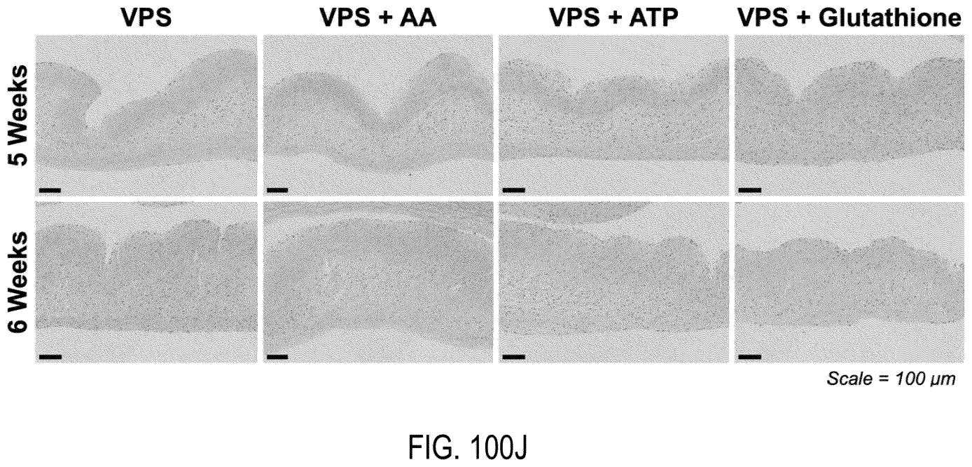

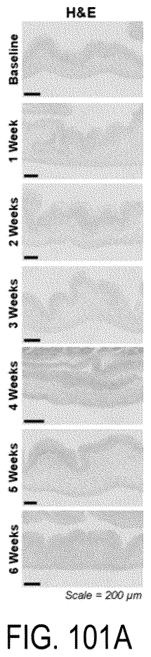

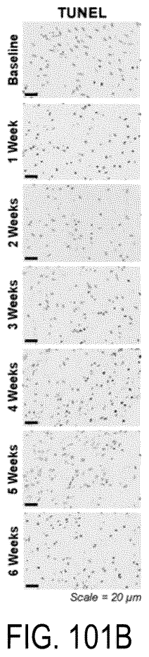

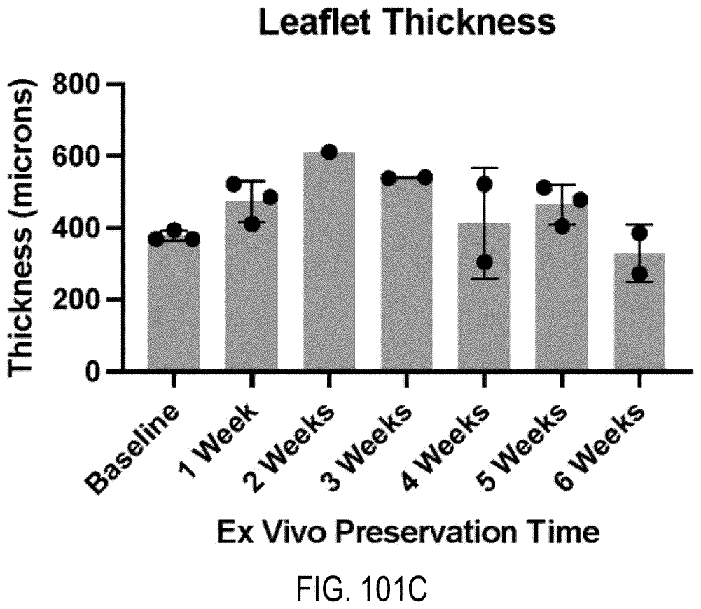

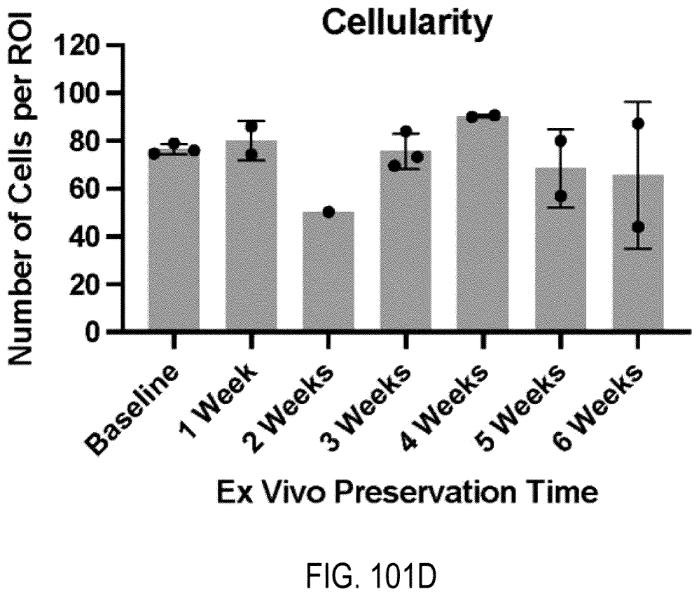

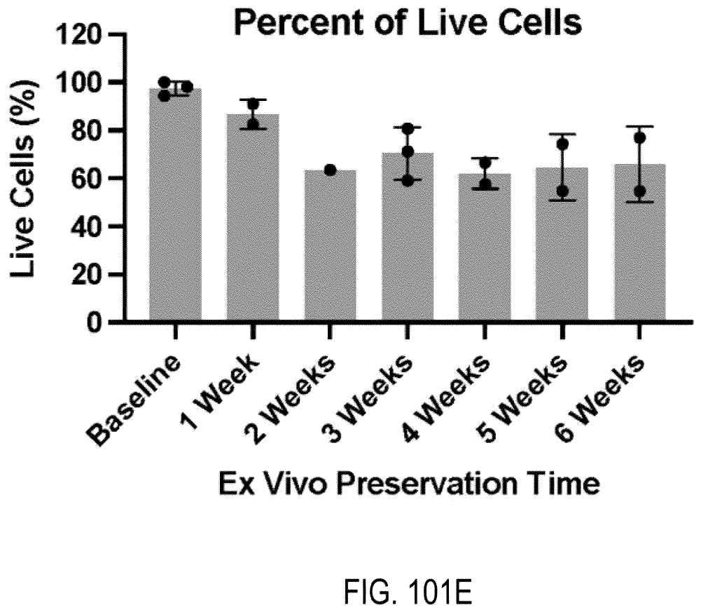

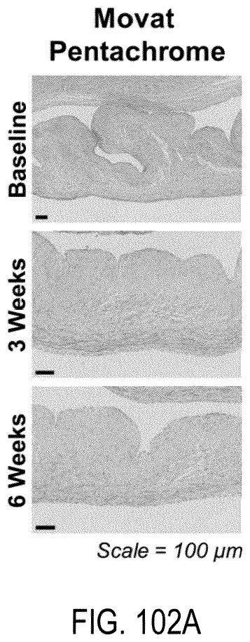

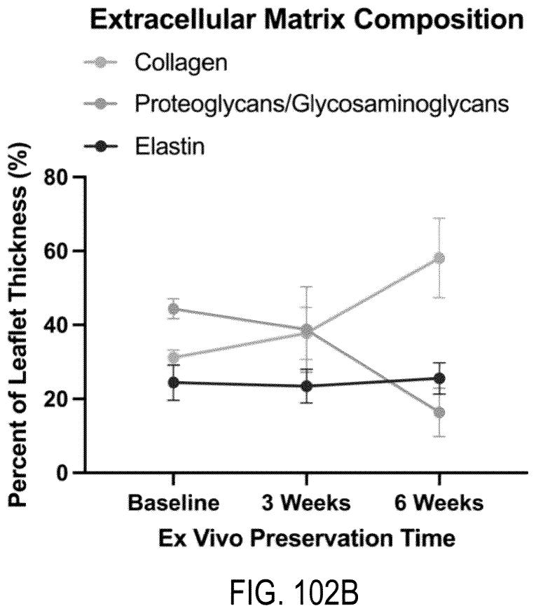

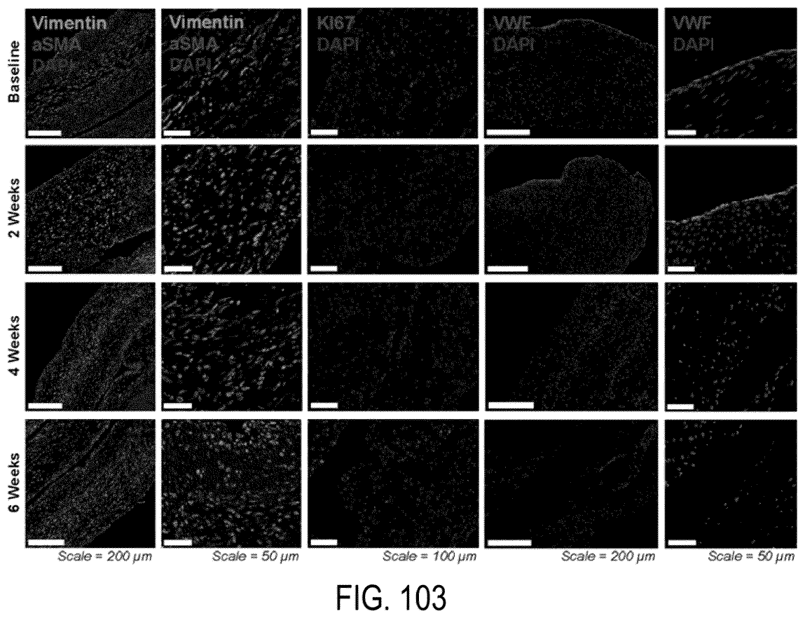

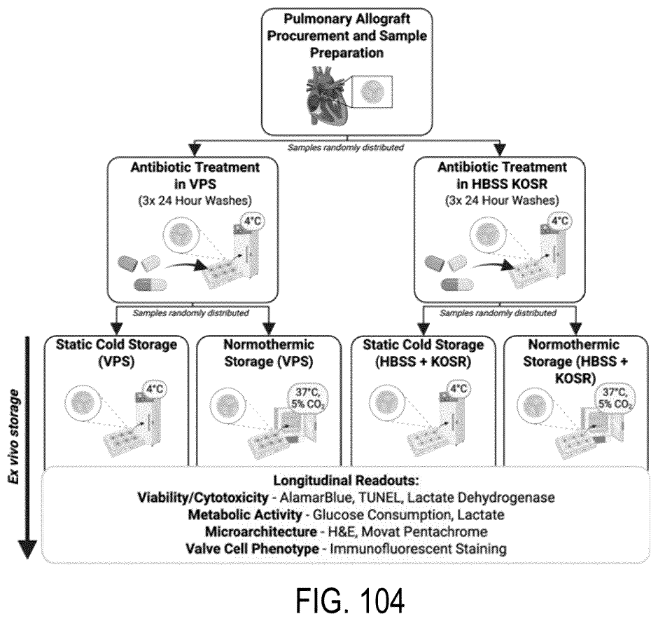

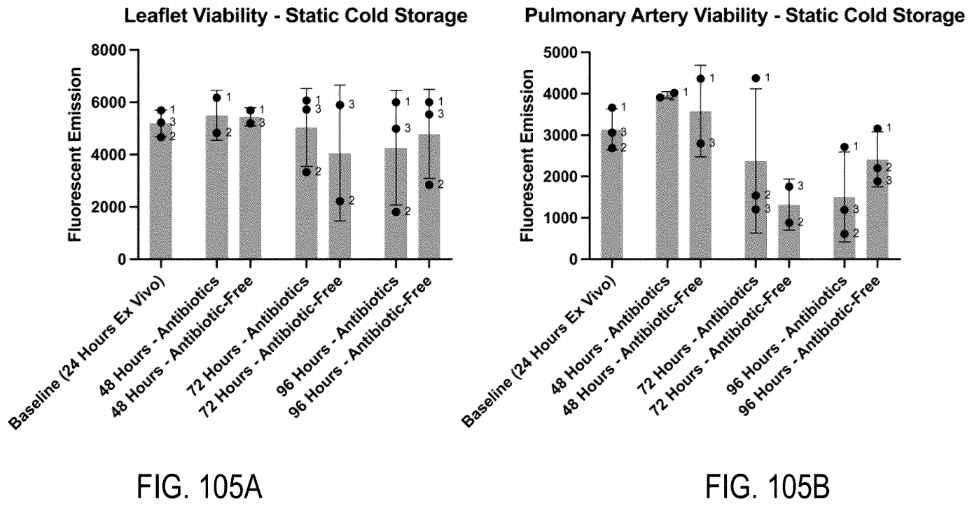

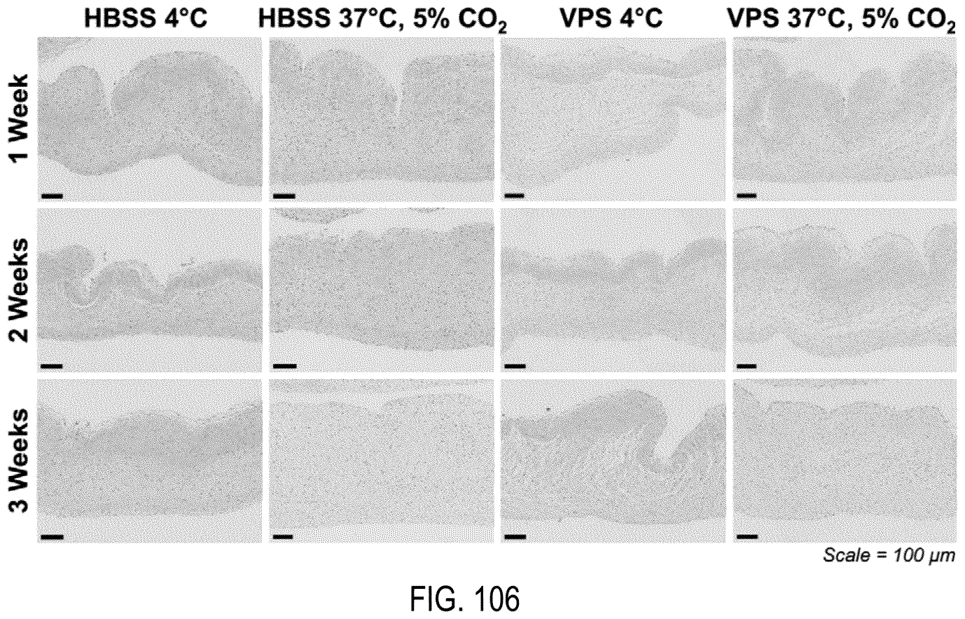

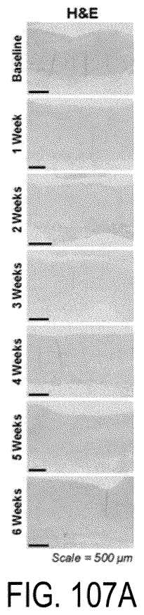

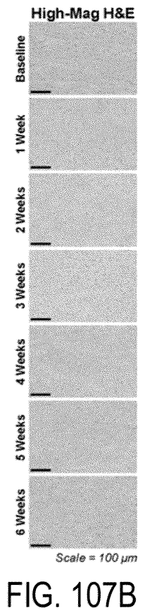

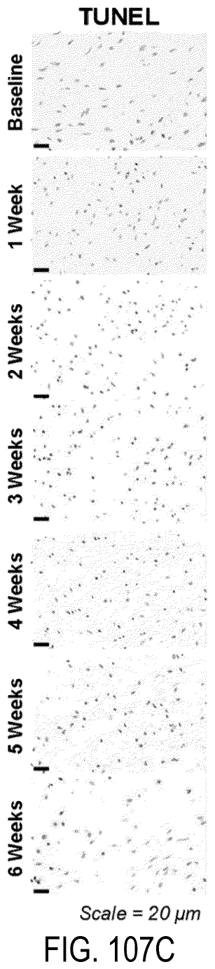

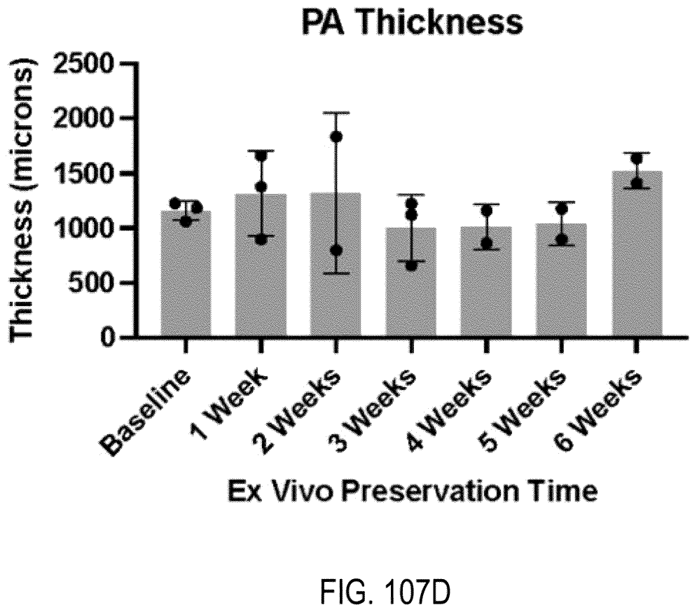

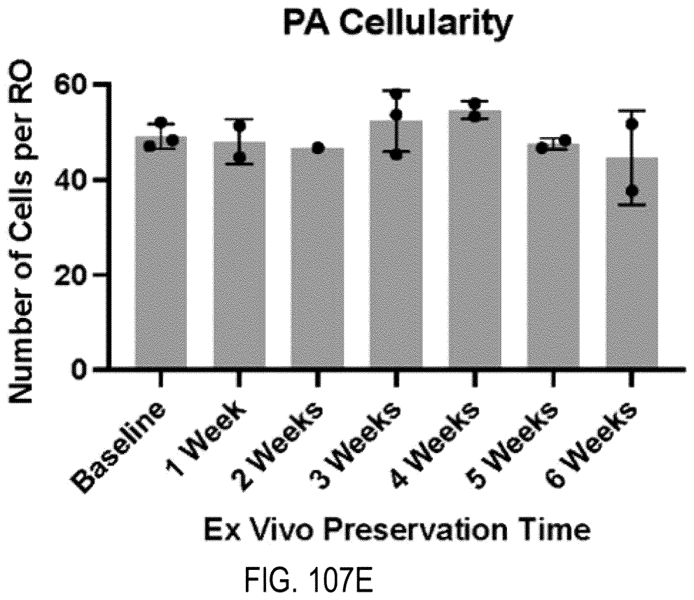

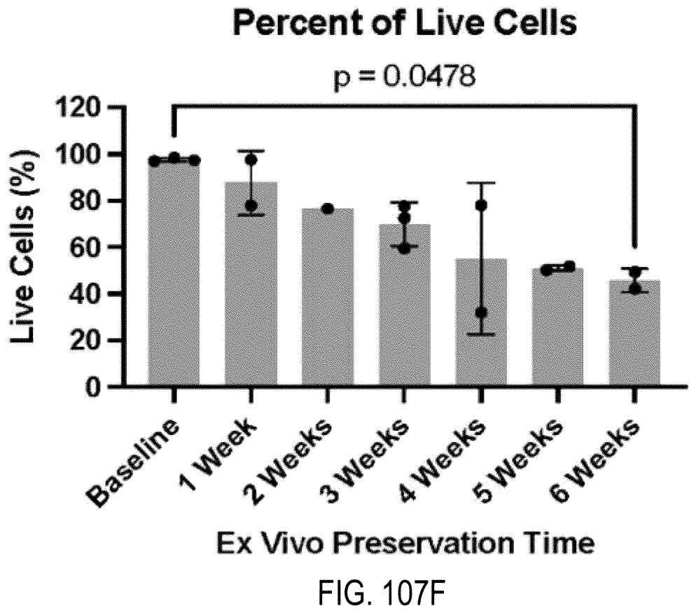

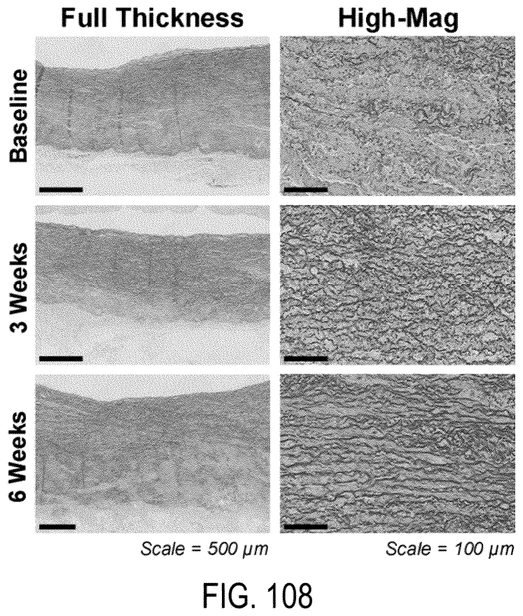

Arachidonic acid (FIG. 100A, 100D), Adenosine 5′-triphosphate (FIG. 100B, 100E), and Glutathione (FIG. 100C, 100F) were added to Valve Preservation Solution throughout the culture period to assess their effect on longitudinal valve viability. None of these additives significantly improved the longitudinal viability of leaflet (FIG. 100A-100C) or pulmonary artery (FIG. 100D-100F) tissue. For FIG. 100A-100F, 2-3 technical replicates were obtained for each datapoint, with N=2-20 biological replicates per group and timepoint. FIG. 100G-100I. No biochemical additives increased glucose consumption throughout storage (marker of metabolic activity). For FIG. 100G-100I, there were N=2-20 tissue samples per group and timepoint. FIG. 100J. H&E cross-sections through the valvular leaflet for each condition at 5- and 6-weeks ex vivo (representative images from N=2-3 biological replicates per group and timepoint). For FIGS. 100A-100F, mean+/−SD are shown. Statistics were Kruskal-Wallis test with Dunn's multiple comparisons test comparing each longitudinal timepoint to the first-available value. Fresh valve samples are the same in FIG. 100A-100C and FIG. 100D-100F (shared with FIGS. 2 and 3). VPS=Valve Preservation Solution: AA=Arachidonic Acid: ATP=Adenosine 5′-triphosphate. H&E=Hematoxylin & Eosin.

FIGS. 101A-101E: Histologic analysis of leaflet viability and microarchitecture. FIG. 101A. Representative H&E cross-sections through the leaflet demonstrating preservation of tissue microarchitecture through approximately 3 weeks ex vivo, at which point the leaflet trilaminar architecture begins to degrade. FIG. 101B. Representative leaflet TUNEL stains. FIGS. 101C-101D. Leaflet thickness and cellularity are preserved throughout storage. E. The percent of live cells decreases in the first 2 weeks ex vivo, with tissue viability preserved at 70% thereafter. Representative images are shown from N=1-3 biological replicates per group and timepoint. For FIGS. 101C-101E, mean+/−SD are shown: N=1-3 biological replicates per timepoint. Statistics were Kruskal-Wallis test with Dunn's multiple comparisons test comparing each longitudinal timepoint to baseline. H&E=Hematoxylin & Eosin.

FIGS. 102A-102B: Leaflet extracellular matrix composition throughout storage. 102A. Movat Pentachrome staining highlighting the relative preservation of collagen (yellow), glycosaminoglycans (blue) and elastin (black) throughout the valvular leaflet up to 3 weeks ex vivo. 102B. Extracellular matrix composition was quantified at baseline, and 3- and 6-weeks ex vivo. Between 3 and 6 weeks of storage, glycosaminoglycans are lost and the valvular leaflet becomes primarily collagen-based, with physiologic elastin present in the ventricularis layer. Mean+/−SD are shown, N=2 biological replicates per timepoint.

FIG. 103: Leaflet cell phenotype throughout storage. Immunofluorescent staining of the leaflet cross-section showing preservation of leaflet Vimentin expression and lack of alpha-Smooth Muscle Actin expression, relative to baseline. Valve interstitial cells retain a quiescent phenotype, without significantly upregulated proliferation (KI67). Valve endothelial cells (VWF) are preserved through 2 weeks ex vivo. Representative images are shown from N=2 biological replicates per group and timepoint. aSMA=alpha-Smooth Muscle Actin. VWF=von Willebrand Factor.

FIG. 104: Experimental design. Upon receipt, pulmonary allografts were dissected into discrete samples. All samples were treated with an antibiotic cocktail of Vancomycin, Lincomycin, and Polymyxin in either Valve Preservation Solution or Hanks Balanced Salt Solution with Knock Out Serum. After three 24-hour washes in the antibiotic solution, samples were evenly and randomly distributed to their experimental condition for longitudinal storage (either cold or normothermic static storage).

FIGS. 105A-FIG. 105B: Short-term valvular allograft preservation in static cold storage. FIG. 105A. Valvular leaflets tend towards preserved viability following 96 hours ex vivo (72 hours of antibiotic treatment post-tissue receipt). FIG. 105B. Pulmonary artery tissue tends towards reduced viability within 72 hours ex vivo (48 hours post-tissue receipt), irrespective of whether antibiotics were included in the solution. Mean+/−SD are shown, with 3 technical replicates obtained for each datapoint: N=2-3 biological replicates per group and timepoint (each donor labeled). Statistics were Kruskal-Wallis test with Dunn's multiple comparisons test comparing longitudinal columns to baseline.

FIG. 106: Histologic tissue preservation in normothermic versus static cold storage. Tissue microarchitecture demonstrates interstitial edema by 3 weeks ex vivo. In HBSS at 37° C. (5% CO₂), complete loss of the trilaminar architecture is observed by 3 weeks ex vivo. In VPS at 37° C. (5% CO₂), the trilaminar architecture begins to degrade at 3 weeks ex vivo. Representative images are shown from N=2-3 biological replicates per group and timepoint. VPS=Valve Preservation Solution. HBSS=Hanks Balanced Salt Solution.

FIGS. 107A-107F: Histologic analysis of pulmonary artery viability and microarchitecture. FIG. 107A-FIG. 107B. Representative full thickness and high-magnification H&E cross-sections through the pulmonary artery. FIG. 107C. Representative pulmonary artery TUNEL stains. For FIGS. 107A-107C, representative images are shown from N=2-3 biological replicates per group and timepoint. FIGS. 107D-107E. Pulmonary artery thickness and cellularity are preserved throughout storage. FIG. 107F. The percent of live cells decreases steadily throughout storage, with 70% of cells alive at 3 weeks and 45% of cells alive at 6 weeks. For D-F mean+/−SD are shown: N=1-3 biological replicates per timepoint. Statistics were Kruskal-Wallis test with Dunn's multiple comparisons test comparing longitudinal columns to baseline. H&E=Hematoxylin & Eosin.

FIG. 108: Pulmonary artery extracellular matrix composition throughout storage. Movat Pentachrome staining demonstrating consolidation of elastin fibers throughout the first 3 weeks ex vivo. Representative images are shown from N=2 biological replicates per timepoint.

### DETAILED DESCRIPTION OF ILLUSTRATIVE EMBODIMENTS

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments can be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” can be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

The present disclosure provides systems and methods for preservation and/or rehabilitation of living allogenic heart valves (LAV). Several critical factors have a bearing on the successful preservation and rehabilitation of the living allogenic heart valves. These critical factors include temperature cues, biochemical cues and mechanical cues. Embodiments of the systems and methods of the present disclosure account for each of these critical factors.

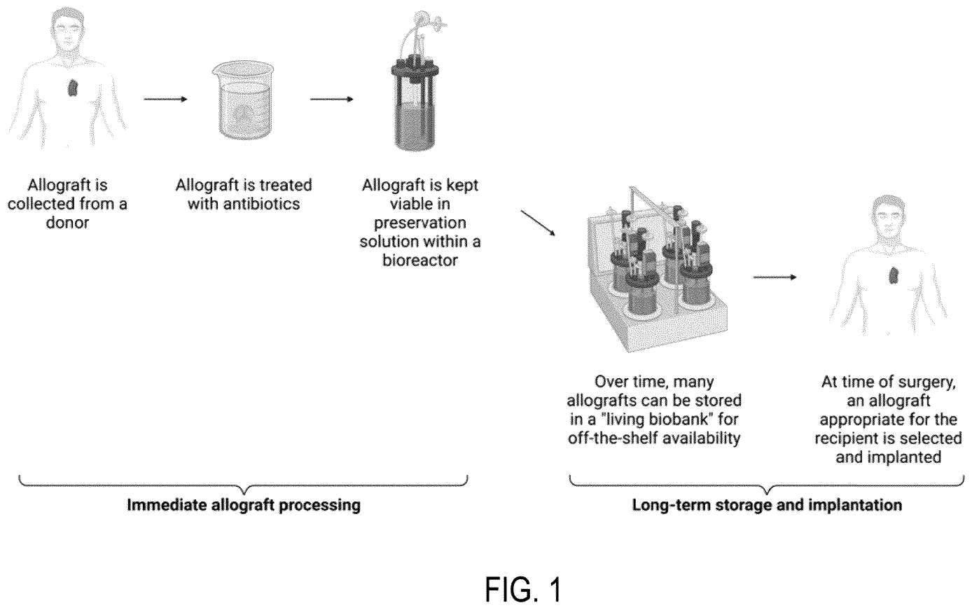

As illustrated in FIG. 1, the present disclosure provides novel systems and methods for processing heart valve allografts. The systems and methods provide for immediate allograft processing for improving tissue viability. The systems and methods also provide for long term allograft storage and implantation into a recipient. The heart valve allografts can be kept viable in long-term storage providing an “off-the-shelf” source of living valve replacements. In some embodiments, the living allogenic heart valve replacements (LAV) are capable of growth and repair. Possible sources of tissue used in LAVs includes: healthy valves from hearts deemed unsuitable for transplant (cardiomyopathy, ischemic time, etc.), and healthy valves from patients receiving heart transplants.

As illustrated in FIG. 2, bioprosthetic values (BP) are associated with high reoperation rates. For example, patients aged 50 at the time of valve implantation may experience a lifetime reoperation risk of over 40%, with bleeding risks over 10%. Mechanical prosthesis (MP), while exhibiting a lower reoperation risk, require life-long use of anticoagulants, and have associated bleeding risks from 30% for patients aged 50 to over 50% for patients aged 75. Despite its prevalence and mortality, no safe long-term treatment exists for irreparable valve disease.

Current treatment for congenital valve disease likewise includes the use of mechanical valves and tissue-based valves, having the draw backs noted herein. Since neither valve replacement is capable of growth or remodeling, young patients are subject to (at times multiple) reoperations.

As illustrated in FIG. 3, patients having valve replacement surgery experience decreasing freedom for homograft failure in subsequent years from surgery. The decline is especially marked in younger patients less than one year old at the time of valve replacement. Thus, young patients with irreparable valve disease are especially vulnerable to failed valve replacements and subsequent reoperation.

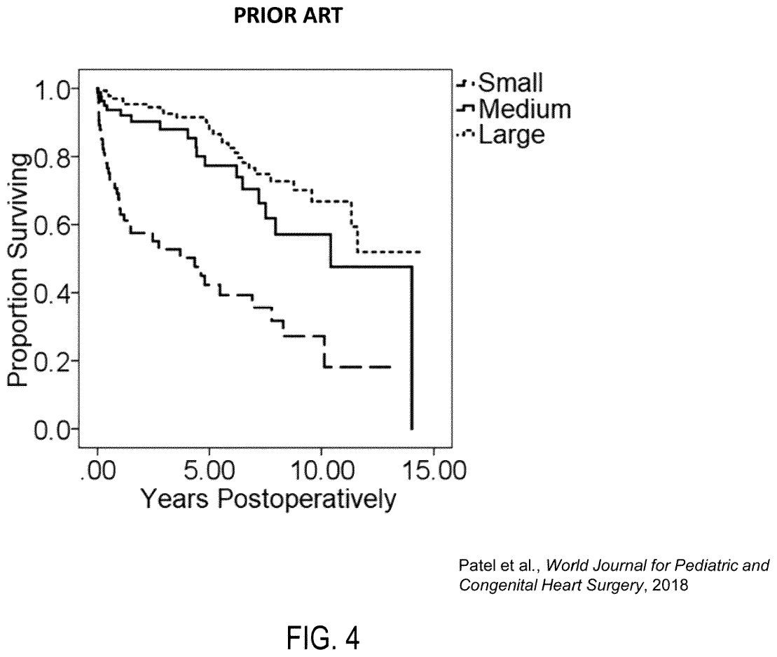

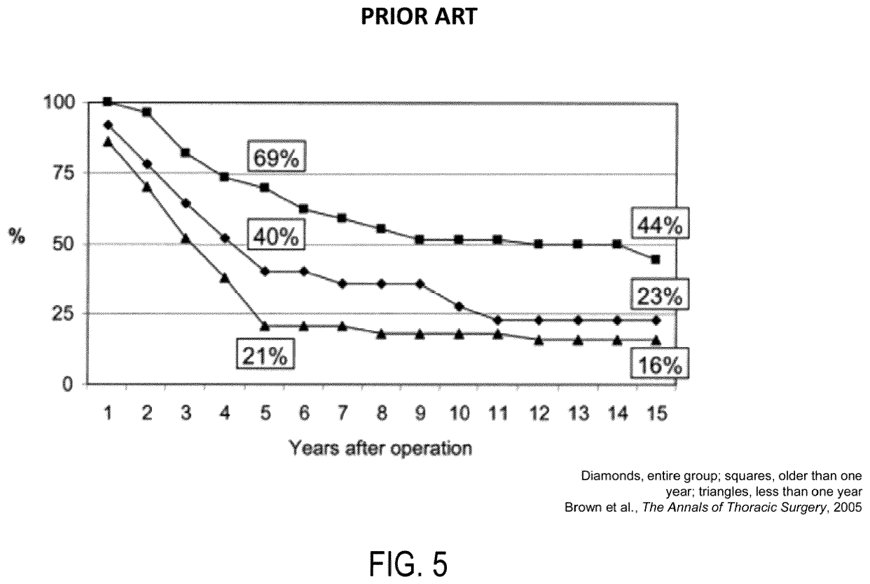

Younger patients receiving smaller grafts are at highest risk of reintervention or reoperation following a tissue-based valve replacement. The common risk factors for valve dysfunction and failure include smaller valves and conduit size, younger age at the time of surgery, and the complexity of congenital heart disease. Common failure modes include calcification and fibrosis. FIG. 4 illustrates the declining proportion of pediatric patients with bovine conduits surviving postoperatively, with lowest rates of survival for small conduits (12-14 mm). FIG. 5 illustrates declining freedom from pulmonary valve dysfunction for cryopreserved homografts, stabilizing after the first five years after operation to 21-16% for patients less than one year old at the time of surgery.

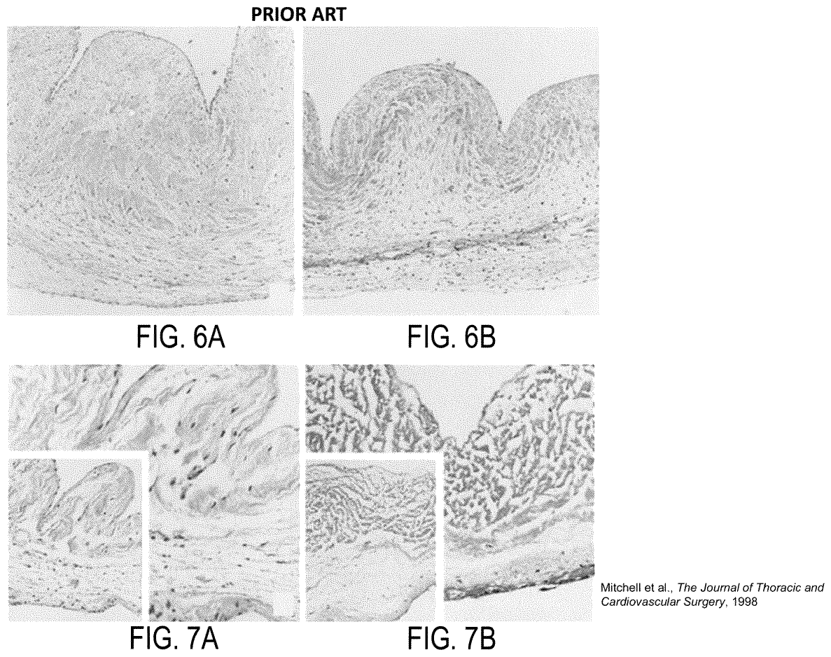

Cryopreservation significantly reduces homograft cell viability and alters tissue architecture. Whereas “fresh” cardiovascular valve tissue processed immediately exhibited viability of 48±3%, cryopreserved cardiovascular valve tissue frozen for 3 weeks exhibited viability of 8±1%. FIGS. 6A-6B are photomicrographs of cusps from valves from orthotopic heart transplants, both showing the expected layered architecture, as well as a normal complement of interstitial and endothelial cells (Hematoxylin and eosin (H&E), original magnifications 90×). FIG. 7A illustrates un-implanted cryopreserved valve tissue, and FIG. 7B illustrates cryopreserved explant removed at autopsy 2 days after implantation. FIGS. 7A-7B show reduced interstitial cellularity (Hematoxylin and eosin stain, original magnification 1753: insets 903).

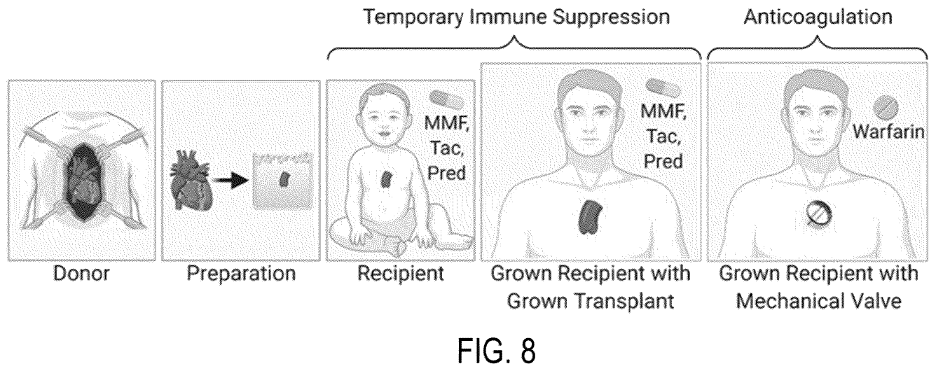

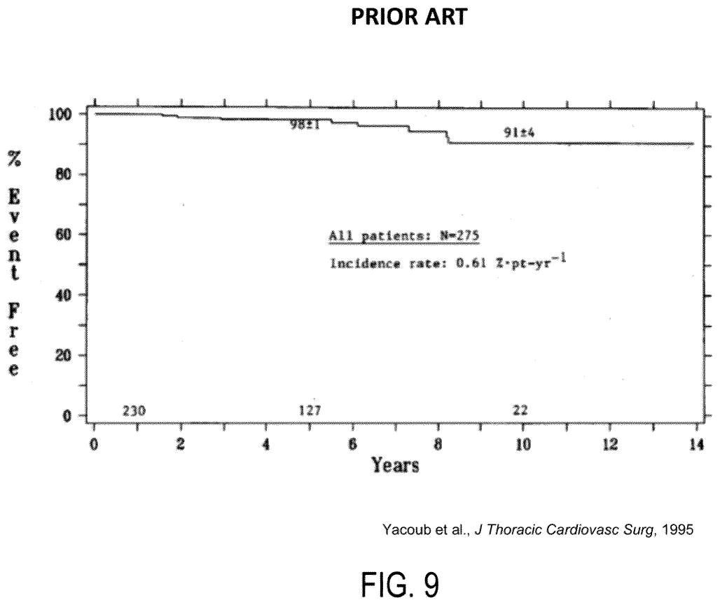

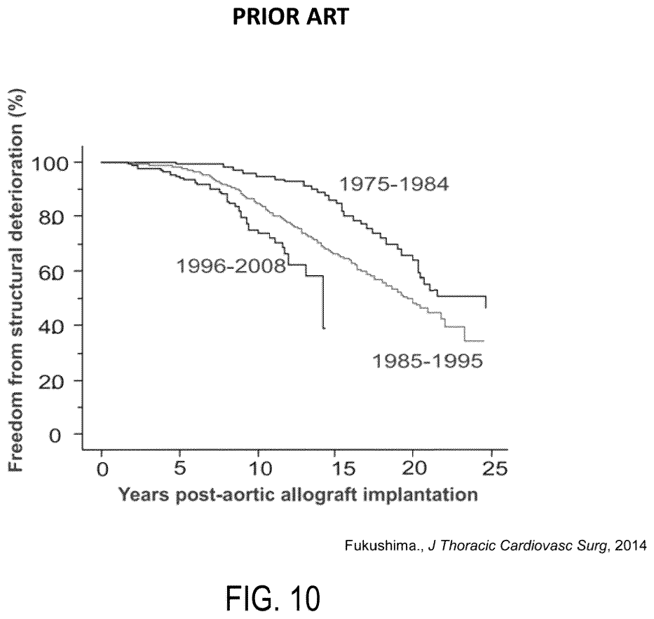

Partial heart transplant shows promise as a means of offering living valvular homograft. FIG. 8 illustrates the concept in which a valvular homograft is collected from a donor, and the homograft is prepared for implantation. Once implanted, the recipient is given temporary immunosuppression to prevent homograft rejection. The implanted valvular homograft is capable of growing with the patient. FIGS. 9-10 show that “fresh” homografts demonstrated freedom from structural deterioration matching or surpassing that of cryopreserved homografts.

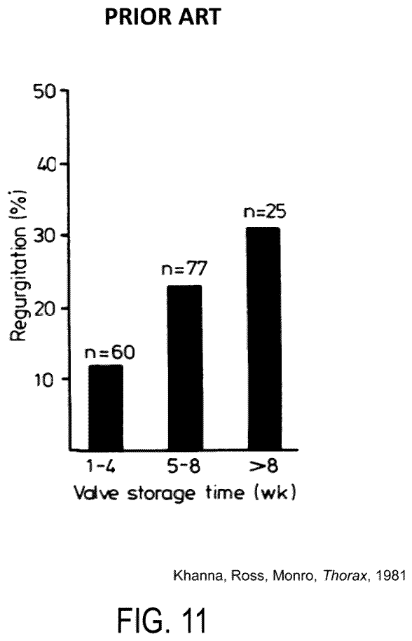

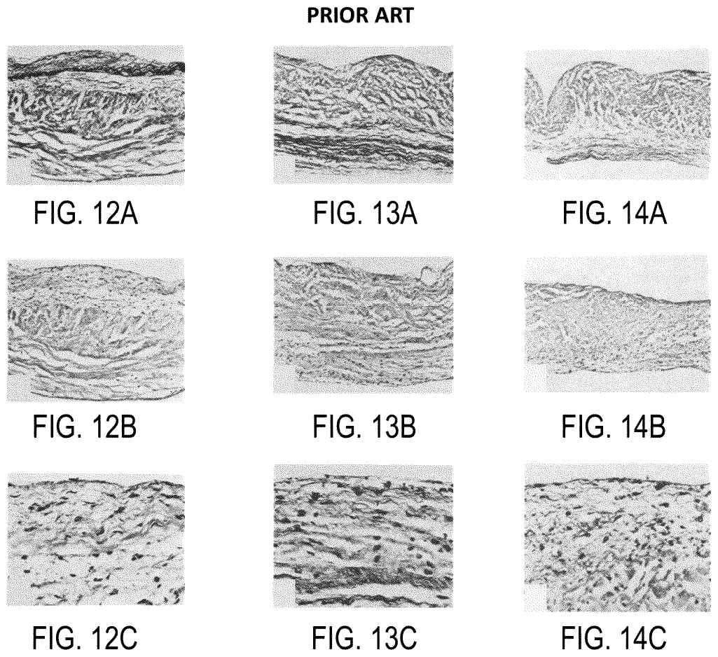

There are recognized shortcomings to the use of “fresh”/“homovital” homographs. Long-term storage of “fresh” homografts is associated with valvular regurgitation and reduced leaflet cellularity. FIG. 11 illustrates the incidence of aortic valve regurgitation as a function of valve storage time. FIGS. 12A-C illustrate the histologic appearance of fresh valves prior to preservation, noting the cellularity of the leaflet within the elastic layer (FIG. 12A) and the integrity of the collagen bands (FIG. 12B). The endothelial layer (FIG. 12C) is intact. FIGS. 13A-13C illustrate the histologic appearance of a valve after 4 weeks of preservation. Cellularity and integrity of layers is similar to the freshly harvested valve shown in FIGS. 12A-12C. FIGS. 14A-14C illustrate the histologic appearance of a valve after 3 months of preservation. There is a slight decrease in cellularity and width of the elastic layer. It was found that definite advantages were realized with the use of “fresh” wet-stored antibiotic-sterilized human homograft valves. However, problems with availability and lack of certainty concerning preservation and storage techniques limited their widespread use. The combination of their resistance to infection, excellent hydraulic function, absence of need for anticoagulation, and versatility in difficult outflow reconstructions made them optimal choices beyond the single issue of durability.

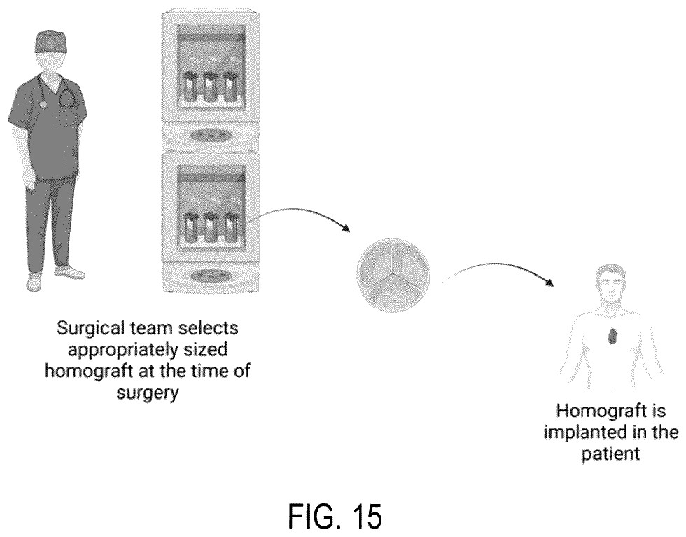

The disclosed subject matter relates to the treatment of a homograft, e.g., a living rehabilitated allogenic heart valve replacement (LAV). As illustrated in FIG. 1, the process includes immediate allograft processing and long-term storage and implantation. The immediate allograft processing involves the collection of the allograft from a donor, treating the allograft with antibiotics, and keeping the allograft viable in preservation solution within a bioreactor. For storage and implantation, many allografts can be stored in a “living biobank” for off-the-shelf availability. At the time of surgery, an allograft appropriate for the recipient is selected and implanted. FIG. 15. Heart valve allografts can be kept viable in long-term storage, providing an “off-the-shelf” source of living valve replacements capable of growth and repair, and rehabilitated using biological, biochemical, biomechanical and immunomodulatory agents in order to increase their viability, durability, availability, growth potential and overall performance.

The techniques described herein have a number of advantages, for example, LAVs are viable valvular tissue capable of growth and remodeling: a large donor pool increases valve availability: valve allografts are available “off-the-shelf” in a variety of sizes, for rapid access at time of surgery. Further advantages are set forth in Table A below.

TABLE A

| | “partial heart | | | |
|---|---|---|---|---|
| | transplant” or | | | Disclosed approach - living |
| | “valve allograft | Homovital | Cryopreserved | rehabilitated valve allograft |
| | transplant” | homograft | homograft | (LAV) |
| Donor | Living Donor | Deceased | Deceased | Living or deceased (increased |
| | | | | availability) |
| Transplant-related | Yes | No | No | no |
| complex logistics | | | | |
| Immunosuppression | Yes | Not done | Not done | May not be needed thanks to |
| used | | | | immunomodulatory rehabilitation |
| | | | | (and the use of ABO compatible |
| | | | | allografts) |
| Regulatory | Complex | Complex | Standardized | standardized |
| Off the shelf | No | No | Yes | Yes |
| availability in term of | | | | |
| numbers and sizes | | | | |
| (possibility to create at | | | | |
| bank with a wide size | | | | |
| availability) | | | | |
| Growth and self repair | Yes | ? | No | Yes |
| Living tissue/viable | Yes | Yes ? | No | Yes |
| cells | | | | |
| Extended storage time | No | No | Yes | Yes |
| Extended | No | No | No | Yes |
| death/harvest time | | | | (rehabilitation strategy) |
| Biological and | No | No | No | Yes |
| mechanical | | | | |
| rehabilitation | | | | |
| Reproducibility, | No | No | Yes | Yes |
| safety, and quality | | | | |
| control | | | | |
| Processing adapted to | No | No | No | Yes |
| the age, origin and | | | | |
| type of the tissue | | | | |
| ABO matching | Can be done | Was not | Usually not | Can be done |
| | | done | done | |
| Donor pool | Small | Large | Large | Large - larger if can extend |
| | | | | harvest time with a rehabilitation |
| | | | | strategy |
| Cost efficiency | Cost Related | Cost Related | Cost Related | Limited cost related to bioreactor |
| | To Transplant | To Reop | To Reop | (no transplant logistics and no |
| | Logistics | | | expected reop) |
| | (OPO Fee, Jet | | | |
| | Travel Fee, | | | |
| | Etc.) | | | |

Tissues used in the procedures described herein can be supplied from a number of possible sources. For example, sources include healthy valves from hearts deemed unsuitable for transplant (cardiomyopathy, ischemic time, etc.); healthy valves from patients receiving heart transplants; and valves from all current donors from whom cryopreserved homografts are harvested and the cryopreserved valves from deceased donors vs. fresh cadavers.

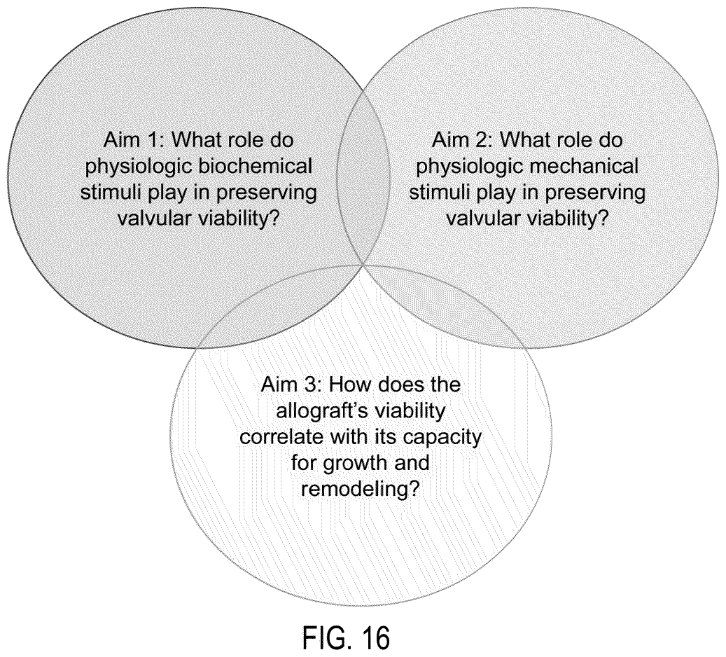

In developing the techniques for preserving a valvular allograft's viability and capacity for growth, several environmental elements can be considered, shown in FIG. 16. These considerations include: First, what role do physiologic biochemical stimuli play in preserving valvular viability? Second, what role do physiologic mechanical stimuli play in preserving valvular viability? Third, how does the allograft's viability correlate with its capacity for growth and remodeling?

### Biochemical Preservation of Allograft Viability

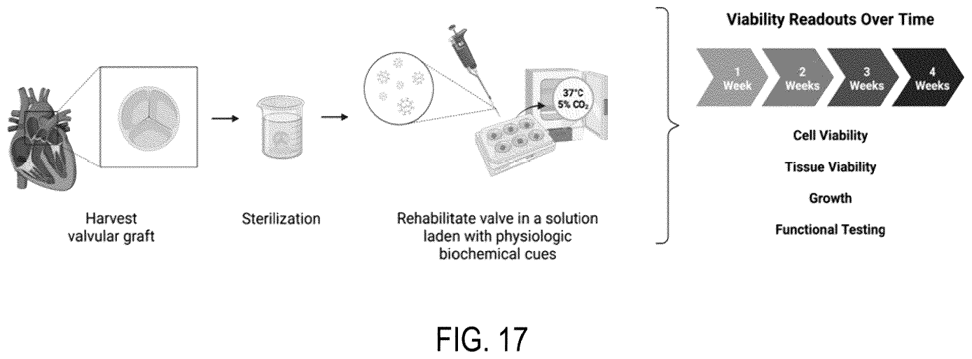

Physiomimetic biochemical cues enable the preservation of healthy valvular tissue. FIG. 17 illustrates the procedure of harvesting the valvular graft, sterilization of the graft and rehabilitation of the valve in a solution laden with physiologic biochemical cues. The introduction of stimuli promoting tissue reperfusion, recovery, and homeostasis promotes the rehabilitation of homografts that have been subject to extended ischemia. Further, the approach capitalizes on storage as an opportunity to reduce the immunogenicity of homografts, by potentially incorporating immune-modulating supplements into the preservation solution. Viability readouts over time include cell viability, tissue viability, growth and functional testing.

A basal preservation solution is used to preserve the tissue. In an exemplary embodiment, a composition includes a base solvent, one or more supplements such as glucose, 1-glucose, dextran, phenol red, sodium pyruvate, HEPES, animal serum, and an animal serum-free formulation configured for cell culture; and one or more antibiotic such as penicillin, streptomycin, vancomycin, imipenem, amphotericin, gentamicin, cefotaxime, fluconazole, polymyxin, and lincomycin. In an exemplary embodiment, the composition further includes a signaling molecule and/or a signaling compound. In an exemplary embodiment, the composition further includes a cell-derived molecule and/or a cell-derived compound, one or more of an immunomodulatory molecule and an immunomodulatory compound, one or more of an antiapoptotic molecule and an antiapoptotic compound, and one or more of a metabolic acid molecule and a metabolic acid compound.

In an exemplary embodiment, the base solvent is Dulbecco's Modified Eagle Medium. In some embodiments, additives and supplements are also used including, Glucose, L-Glutamine, Phenol red, Sodium pyruvate, HEPES, Bovine serum albumin, Fetal bovine serum, KnockOut serum (THERMOFISHER™), CDM-HD (KD Bio), Panexin CD (Ilex Life Sciences), FastGro Synthetic (MP Biomedicals), or an alternative chemically-defined replacement to fetal bovine serum, and Dextran. In some embodiments, basal antibiotics are used, including penicillin, streptomycin, vancomycin, imipenem, amphotericin, gentamicin, cefotaxime, fluconazole, polymyxin, lincomycin.

In another embodiment, the base solvent is Advanced DMEM/F12 with Non-Essential Amino Acids and Sodium Pyruvate. In some embodiments, additives and supplement are used including Knock Out Serum, ascorbic acid, albumin, insulin, L-glutamine (GLUTAMAX), and an antibiotic and antimycotic solution.

The basal preservation solution can include additional supplements, such as signaling factors, hormones, steroids, small molecules; cell-derived materials, anti-apoptotic molecules or compounds: immuno-modulatory molecules or compounds: metabolic aids, and antioxidants; and phenotypic cues. In some embodiments, signaling factors, hormones, steroids, small molecules include fibroblast growth factor 2, vascular endothelial growth factor, bone morphogenic protein 2, retinoic acid, TGFB inhibitors (i.e., SB-431542, SB 525334, SB 505124, dorsomorphin, LY 364947, LY3200882), bone morphogenic protein 1 inhibitors (i.e., K02288, LDN212854), Y-27632, SIRT1 activator 3, muscone, nitric oxide, dexamethasone, hydrocortisone, and/or other corticosteroids, ascorbic acid, L-ascorbic acid 2-phosphate, insulin, thyroid hormone (T3) or other physiological hormones, and caffeine. In some embodiments, cell-derived materials include iPS-derived extracellular vesicles: placenta-derived extracellular vesicles: valve endothelial cell-derived extracellular vesicles: valve interstitial cell-derived extracellular vesicles: cardiomyocyte-derived extracellular vesicles: microRNAs. In some embodiments, immuno-modulatory molecules or compounds include IL-4: IL-6: IL-10: IL-11: IL-13: IL-1 antagonist: bilirubin: carbon monoxide; and mesenchymal stem cell conditioned media. In some embodiments anti-apoptotic molecules or compounds include Necrostatin-1: broad-spectrum caspase inhibitor: individual caspase inhibitors; and autophagy inhibitors. In some embodiments, metabolic aids include ATP, arachidonic acid (AA), supplemental oxygen, and supplemental carbon dioxide. In some embodiments, antioxidants include glutathione. In some embodiments, phenotypic cues include SB431542 (SB). One can provide oxygen supplementation to the composition, for example via one or more of synthetic blood, red blood cells, oxygen carriers, and exogenous oxygen delivery. Additional additives can also include dextran and Albumax™ or other lipid-rich BSA.

### Mechanical Preservation of Allograft Viability

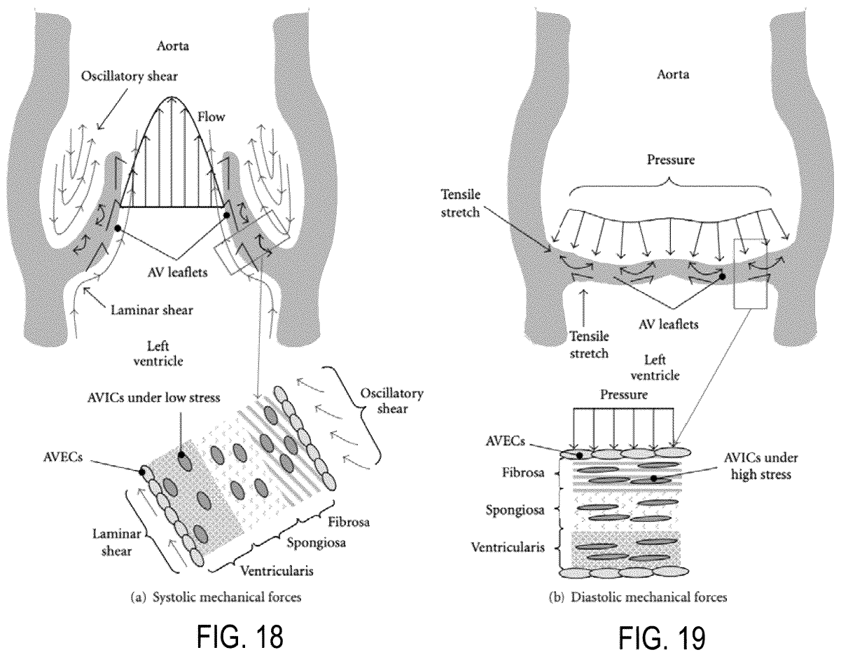

To improve valve tissue viability, native-like mechanical cues are applied to the valve tissue. As illustrated in FIGS. 18-19, valve cells experience laminar and oscillatory shear, as well as bending and tensile stretch throughout open/close cycles. FIG. 18 illustrates the systolic mechanical forces on the valve tissue, and FIG. 19 illustrates the diastolic mechanical forces. This mechanical stimulation is intimately related to valve cell phenotype and morphology, as well as subsequent protein deposition and ECM organization Heart valve leaflets are subject to dynamic stress throughout open/close cycles, which actively sends mechanical signals to resident valve cells, influencing their behavior.

In an exemplary embodiment, the native-like mechanical cues are provided by a pump-less bioreactor. Conventional bioreactors used to recapitulate physiologic open/close cycles are typically low-throughput, and require extensive space and high-cost equipment such as pumps and filters. In an exemplary embodiment, the bioreactor avoids the use of a pump, which can introduce a great deal of bulk and increases the risk of contamination. Further, the bioreactor recapitulates the opening and closing of the valvular homograft at a physiologic rate, and allows for individualized culture of each valve, thus reducing the risk of cross-contamination, and allowing for the removal and inspection of individual homografts.

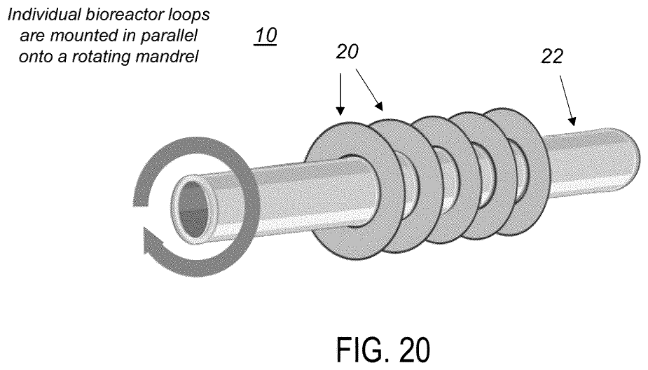

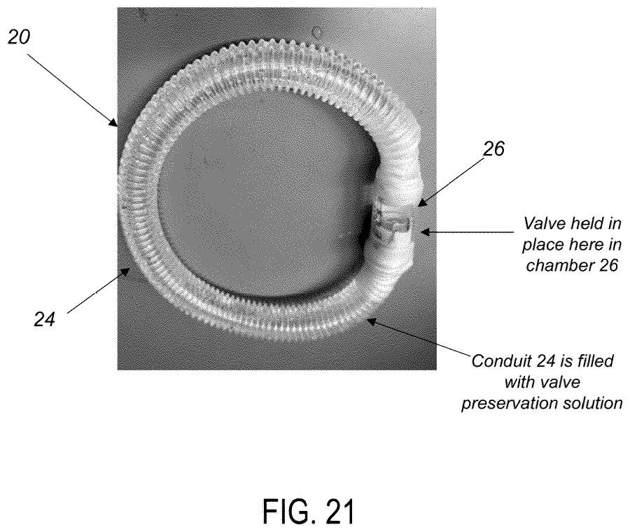

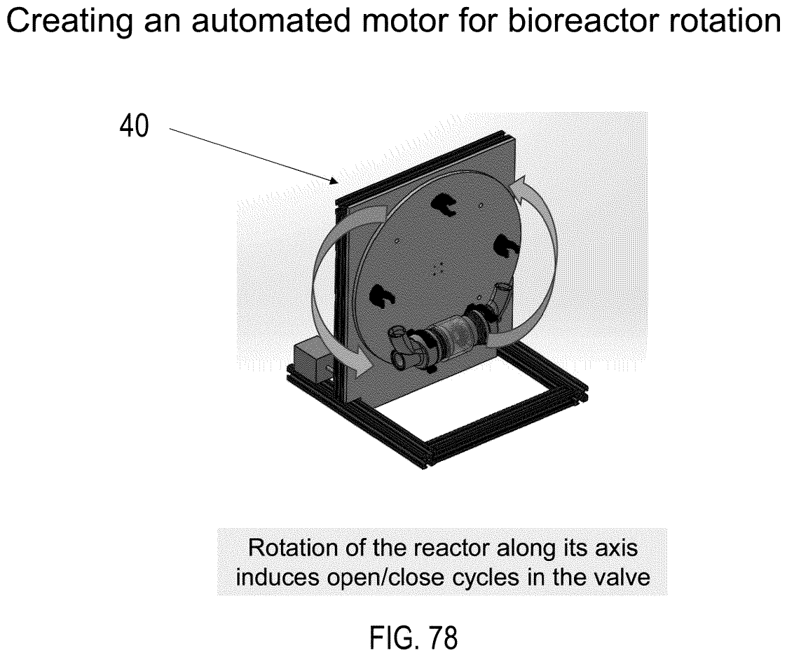

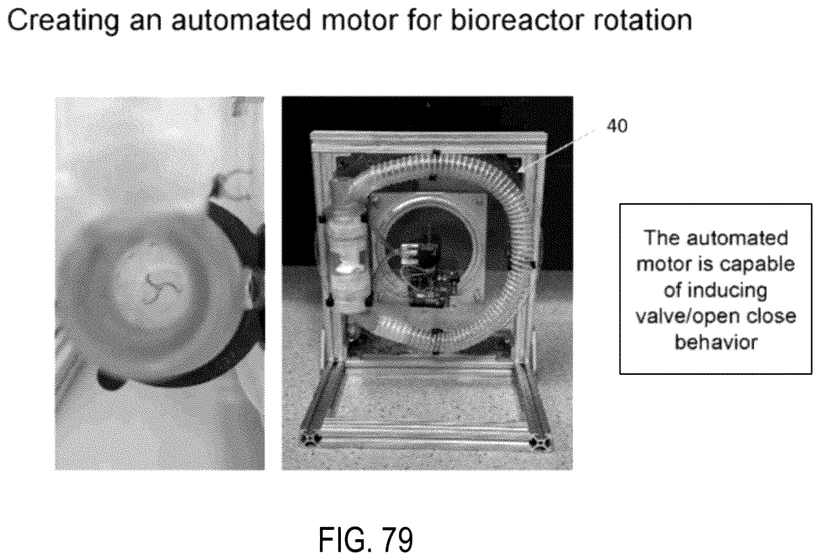

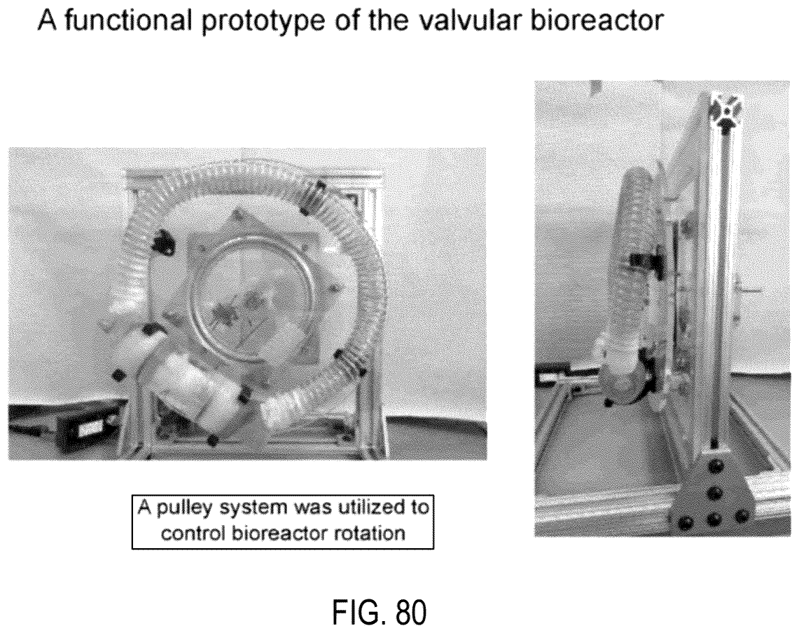

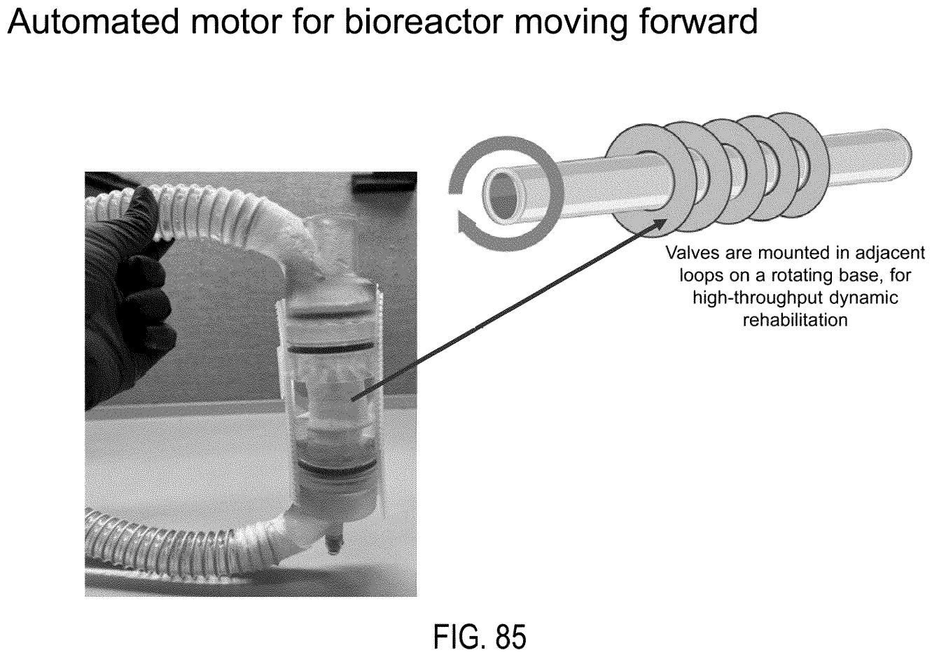

In an exemplary embodiment, the bioreactor 10 includes a “rotating-loop” design that enables a controlled, closed-circuit flow through the valve without the need for a pump. As shown in FIG. 20, the bioreactor system 10 includes a number of bioreactor loops or culture vessels 20 mounted in parallel onto a rotating tubular support or mandrel 22. As shown in FIG. 78, in some embodiments, the bioreactor system 10 includes a number of bioreactor loops or culture vessels 20 mounted in parallel onto a rotating manifold 40. In some embodiments, the mandrel 22 or manifold 40 is driven by a motor directly (shown in FIG. 79) for rotating the bioreactor loop. In some embodiments, the mandrel 22 or manifold 40 is driven by a motor coupled to a pulley system (shown in FIG. 80) for rotating the mandrel 22 or manifold 40. As shown in FIG. 21 and FIG. 85, each bioreactor loop 20 includes a chamber 26 that holds a biological tissue, such as the valve, and a conduit 24 in communication with the chamber 26 to facilitate a flow of a composition, such as the valve preservation solution, through the valve tissue. In this manner, many valves can be mounted and cultured simultaneously. The bioreactor loop or culture vessel 20, including the chamber 26 and the conduit 24, are mounted to the support or mandrel 22. The chamber 26 and the conduit 24 are rotated about an axis of rotation to cause the composition to flow through the valve held in the chamber 26. The chamber 26 and the conduit 24 can have an annular configuration. For example, the conduit 24 extends between a first end and a second end, and each of the first end of the conduit 24 and the second end of the conduit 24 is secured to the chamber 26. The conduit 24 defines a first opening at the first end of the conduit 24 and a second opening at the second end of the conduit 24, and each of the first opening and the second opening are in communication with chamber 26. The chamber 26 and the conduit 24 facilitate unidirectional flow of the composition through the chamber 26.

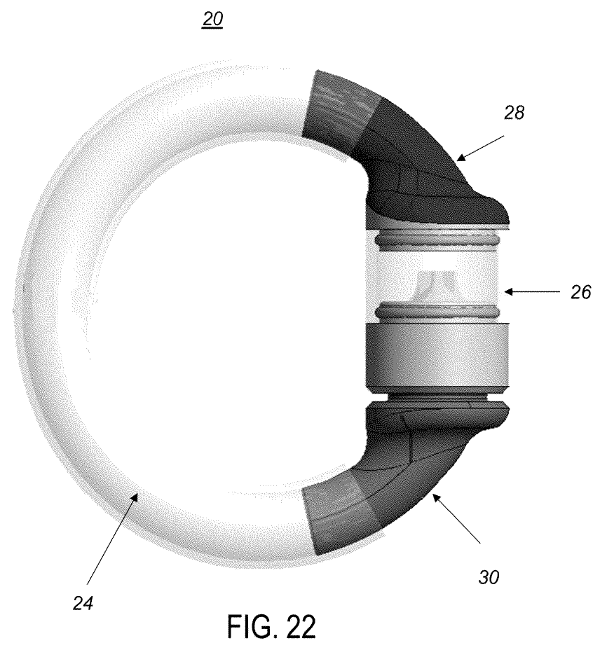

By inducing open/close cycles at a physiologic rate, the rotating loop-bioreactor will provide flow velocities and stress similar to those present in vivo. FIGS. 21 and 22 illustrate an exemplary bioreactor loop or culture vessel 20 in accordance with an exemplary embodiment. The entire loop 20 is mounted onto a rotating mandrel 22.

Movement of preservation solution throughout the loop 20 induces opening/closing of the valve held in the chamber 26. In one embodiment, rotating loop 20 holds ˜200 mL of preservation solution. Adapters 28, 30 connect the valve housing chamber 26 and the conduit 24 to allow the valve to be mounted and removed easily.

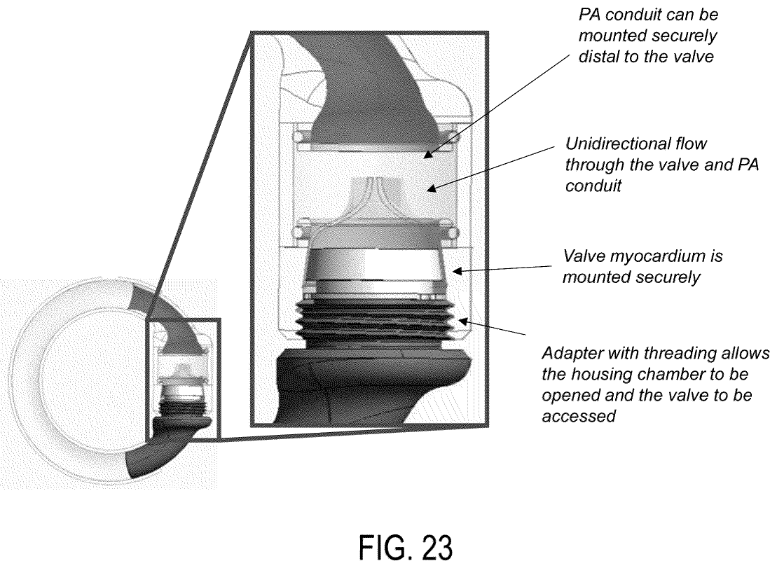

FIG. 23 is an enlarged view of chamber 26. In some embodiments, adapters 28, 30 have threading to allow the chamber 26 to be opened and the valve to be accessed. The valve myocardium is mounted securely to a cylindrical mount. The pulmonary artery (PA) conduit is mounted securely distal to the valve. Unidirectional flow of the preservation fluid passes through the valve and the PA conduit.

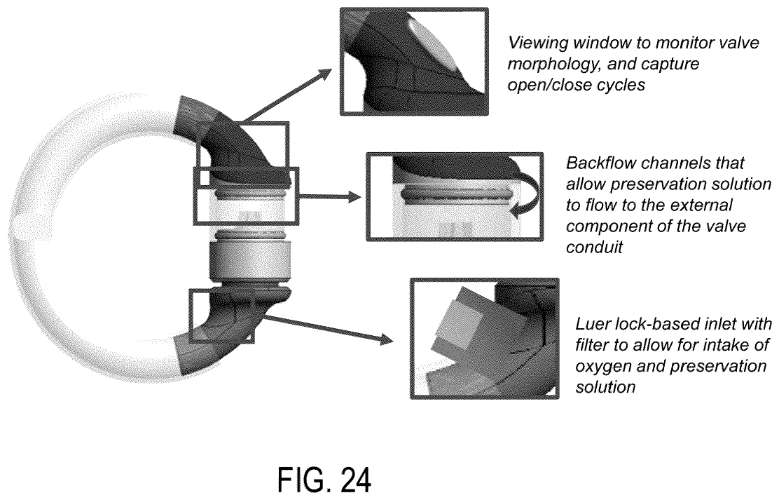

FIG. 24 illustrates additional features of the bioreactor loop 20. One or more of the adaptors 28, 30 are provided with a transparent viewing window to monitor valve morphology and capture open/close cycles. The chamber 26 includes backflow channels that allow the preservation solution to flow to the external component of the valve conduit. One or more of the adapters is provided with a Luer lock-based inlet with a filter to allow for intake of oxygen and preservation solution.

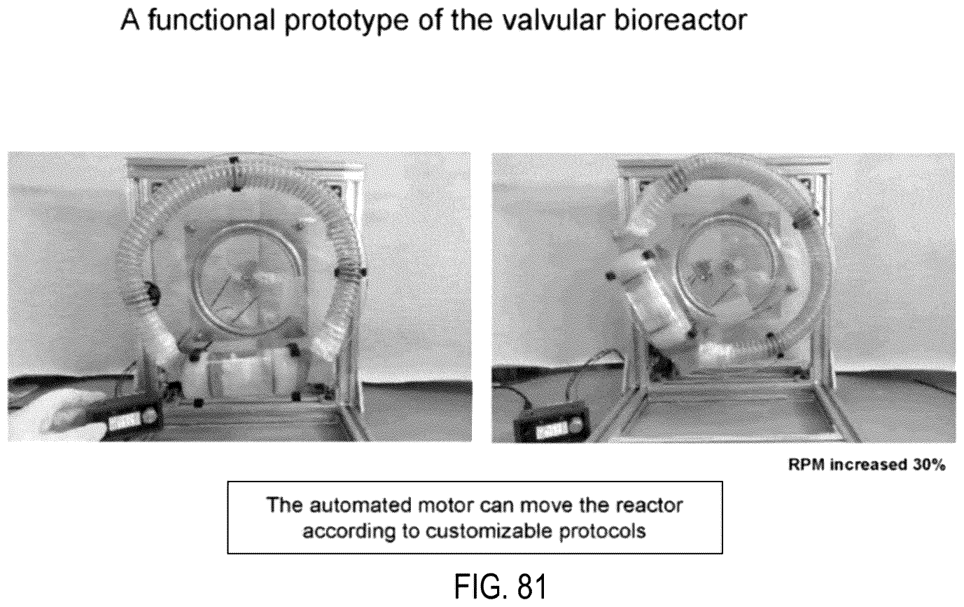

FIG. 78 shows another means for rotating bioreactor loop 20. In some embodiments, bioreactor loop is mounted onto a manifold 40 driven by an automated motor for rotating the mounted bioreactor. In some embodiments, the motor is mounted directly only to manifold for directly inducing rotation. A prototype of the automated motor platform or manifold 40 for inducing bioreactor rotation is shown in FIG. 79. In some embodiments, a pulley system is employed for inducing rotation of the manifold 40 using a motor not directly mounted onto the manifold 40, such as that shown in FIG. 80. In some embodiment, the motor causes rotation of the loop up to 360°. As shown in FIG. 81, in some embodiments, the motor causes rotation of the loop in an oscillating motion. The oscillation can include an oscillation from an initial point of up to +/−10°, of up to +/−15°, of up to +/−20°, of up to +/−25°, of up to +/−30°, of up to +/−35°, of up to +/−40°, of up to +/−45°, of up to +/−50°, of up to +/−55°, of up to +/−60°, of up to +/−65°, of up to +/−70°, of up to +/−75°, of up to +/−80°, of up to +/−85°, of up to +/−90°, of up to +/−95°, of up to +/−100°, including any and all increments therebetween. The oscillating motion can include a frequency in the range of from about 40 cycles per minute to about 180 cycles per minute. Preferred frequencies can include in the range of from about 60 cycles per minute to about 100 cycles per minute.

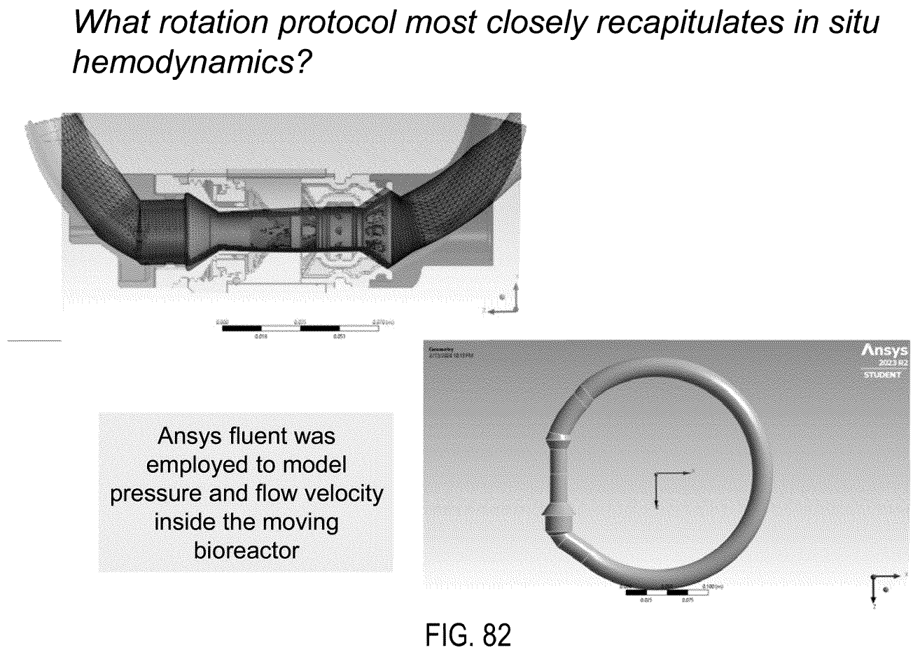

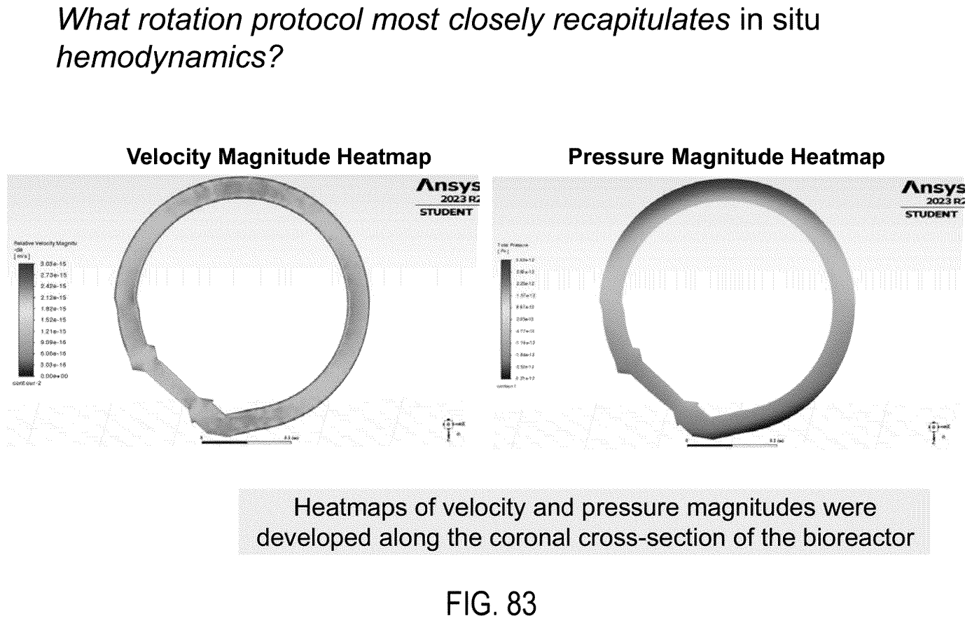

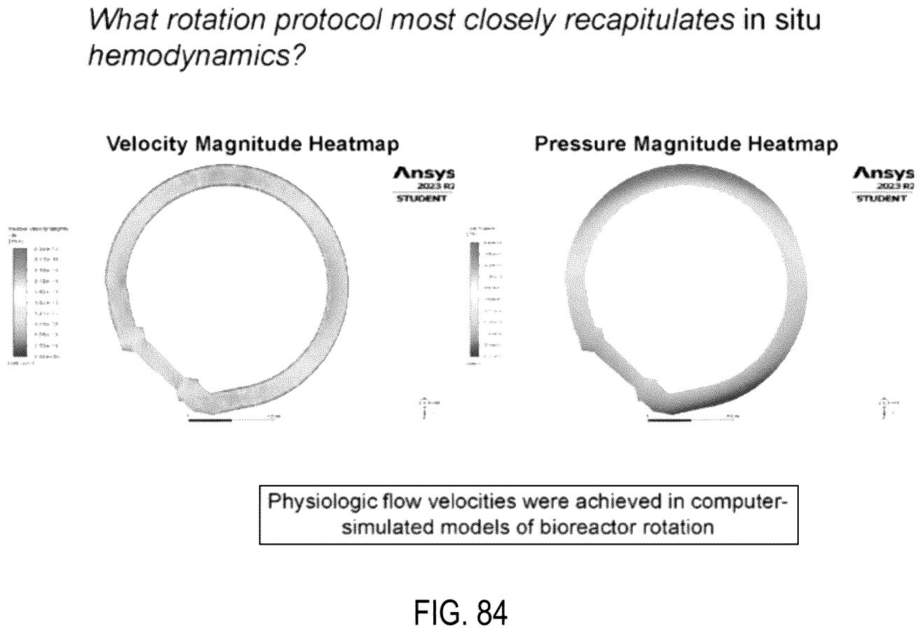

Hemodynamic analysis was performed using ANSYS FLUENT modeling in order to determine whether the bioreactor achieves native-like maximum velocity magnitude and transvalvular pressure values. An exemplary model of the bioreactor loop in shown in FIG. 82. Heatmaps of velocity and pressure magnitudes were developed along the coronal cross-section of the bioreactor, shown in FIG. 83. Physiologic flow velocities were achieved in computer-simulated models of bioreactor rotation, shown in the heatmaps depicts in FIG. 84. Preferred values for maximum velocity magnitude include velocities in the range of from about 0.5 m/s to about 5 m/s. In more preferred embodiments, the velocity magnitude is about 1 m/s. In some configurations, the velocity magnitude can be from about 0) to about 2 m/s. The velocity magnitude can be modulated according to the type of patient: for example, the velocity magnitude used for an adult patient may vary from the velocity magnitude used for a pediatric patient. Preferred values for maximum transvalvular pressure include pressures in the range of from about 10 mmHg to about 100 mmHg. More preferred values for maximum transvalvular pressure are in the range of from about 20 mm Hg to about 60 mm Hg, with more preferred maximum pressure values in the range of from about 30 mmHg to about 50 mm Hg.

### NON-LIMITING EXAMPLES

### Example A

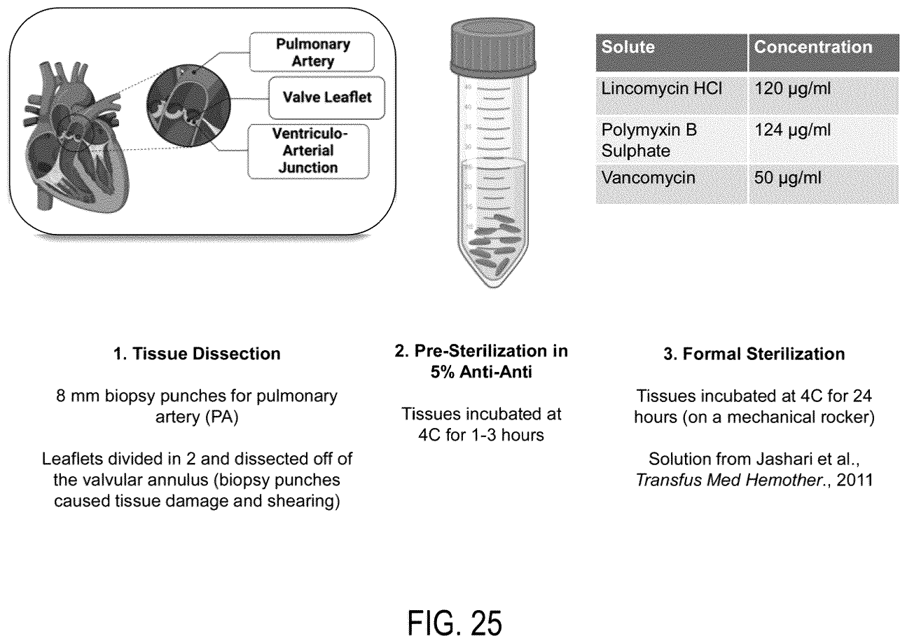

Porcine valve homografts are biopsied and sterilized with antibiotics in preparation for long-term preservation studies. As illustrated in FIG. 25, a protocol for intake and processing of porcine right ventricular outlet (RVOT) tissue includes tissue dissection, pre-sterilization and formal sterilization. During tissue dissection, an 8 mm biopsy punch is used for the PA. The valve leaflets are divided in two and dissected from the valvular annulus. Pre-sterilization solution is used to help eliminate bacteria and fungi before placing the tissues in the formal “antibiotic solution”. In an exemplary embodiment, the tissues are immersed in a solution of 5% Antibiotic/Antimycotic in phosphate buffered serum at 4 C. The 5% Antibiotic/Antimycotic solution contains 10,000 units/mL of penicillin, 10,000 μg/mL of streptomycin, and 25 μg/mL of Gibco Amphotericin B (commercially available at THERMOFISHER™). The tissue is incubated at 4° C. for 1-3 hours. During formal sterilization, the tissues are incubated at 4° C. for 24 hours, e.g., on a mechanical rocker. An exemplary antibiotic solution includes Lincomycin HCl at 120 L/ml concentration, Polymyxin B Sulphate at 124 μL/ml concentration, and Vancomycin at 50 μL/ml concentration.

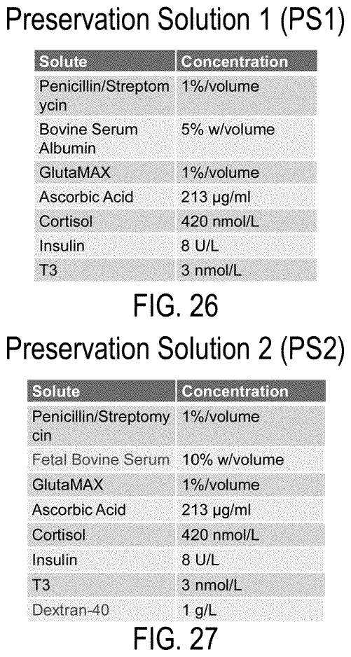

Two preservation solutions were tested, both at 37° C. (incubator) 4° C. (refrigerator). The first preservation solution (referred to herein as PS1) is noted in FIG. 26 and the second preservation solution (referred to herein as PS2) is noted in FIG. 27. Significantly, PS2 replaces bovine serum albumin with fetal bovine serum, and adds Dextran-40.

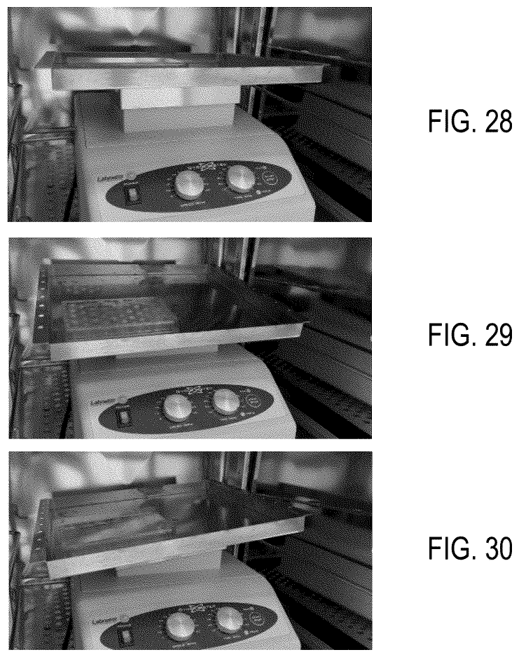

In this example, “mechanical stimulation” of tissues refers to long-term culture on a mechanical rocker (see FIGS. 28-30). Incubated samples were placed on a gyroscopic rocker at a speed of 50-60 cycles/min Fridge-stored samples were placed on a traditional rocker at a speed of 50-60 cycles/min.

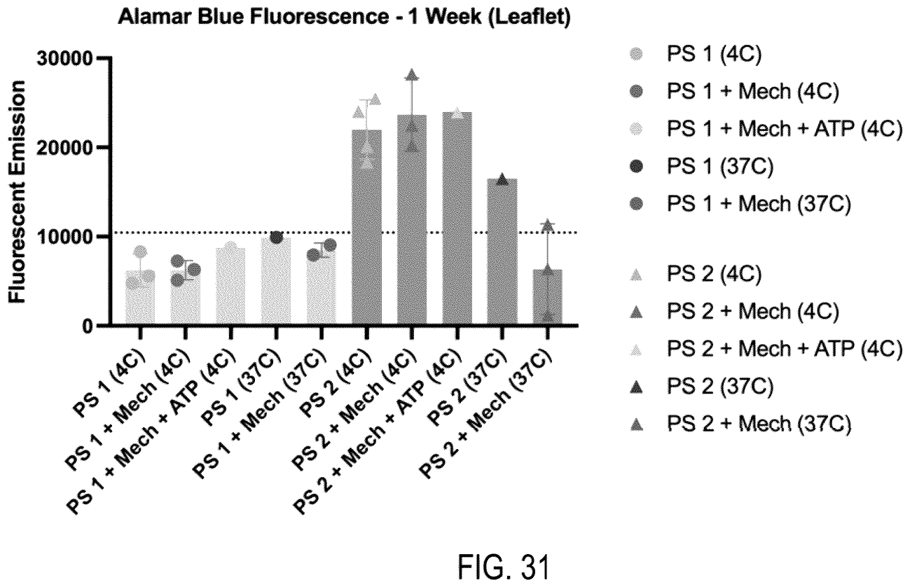

FIG. 31 illustrates the results of whole leaflet viability testing after one week of storage. Samples were tested with PS1 and PS2. Some samples included mechanical stimulation (mech) and ATP. In an exemplary embodiment, the mechanical stimulation is culturing the tissues on an orbital rocker (FIGS. 28-30) set to a speed of 60 revolutions per minute. ATP refers to supplementation of the preservation solution with 60 μM concentration of Adenosine Triphosphate (commercially purchased from Millipore Sigma). Some samples were stored at 37° C., and some samples were stored at 4° C. Samples within each group come from different porcine donors (1-4 technical replicates per data point). (N>1, Mean+/−standard deviation. Dotted line represents baseline leaflet tissue viability (n=1). Results normalized to the fluorescent emission of tissue-free media.) Viability was assessed via Alamar blue fluorescence assay. Higher fluorescence values correspond to increased tissue viability. Data after one week of storage found that PS2 improves leaflet viability.

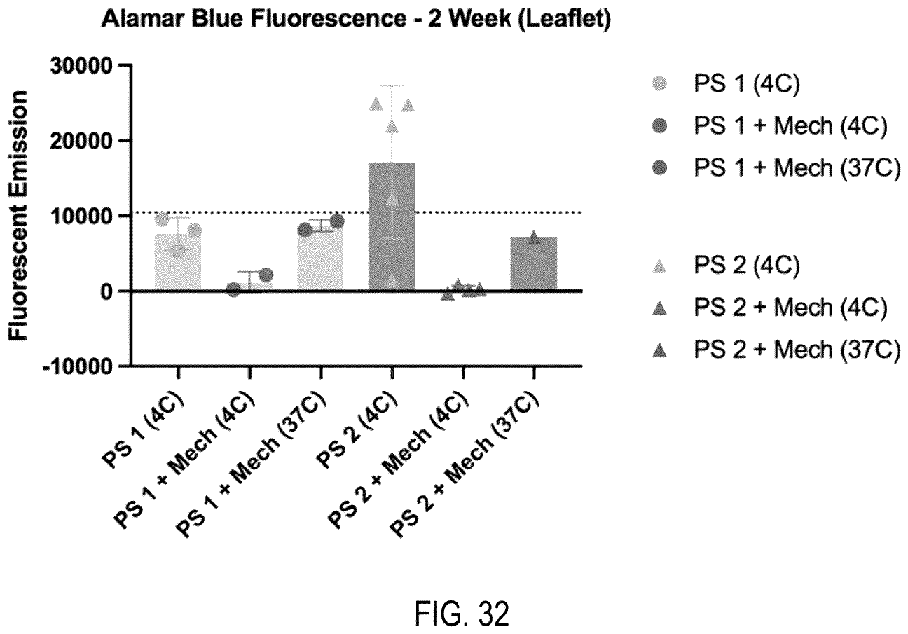

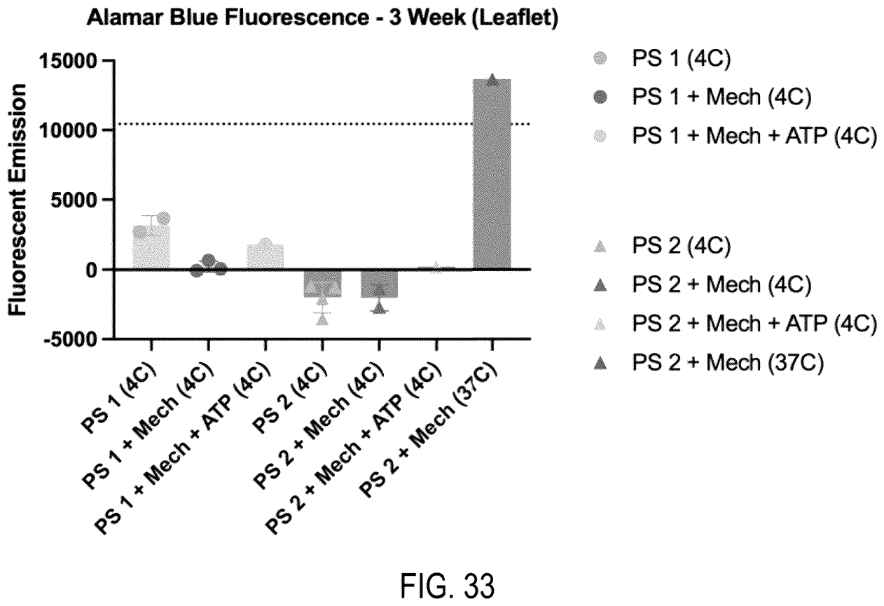

FIG. 32 illustrates the results of whole leaflet viability testing after two weeks of storage. Testing showed that PS1 and PS2 were able to maintain valve leaflet viability up to 2 weeks in storage. FIG. 33 illustrates the results of whole leaflet viability testing after three weeks of storage. Three week-storage was found to be associated with a reduction in whole-tissue viability, prompting incorporation of proposed additives as described hereinabove into the preservation solution.

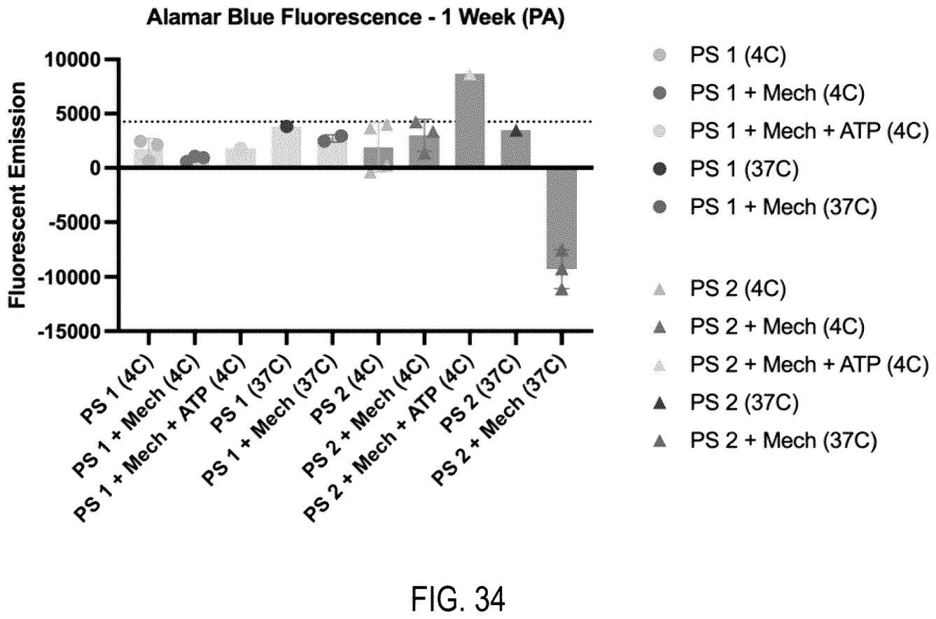

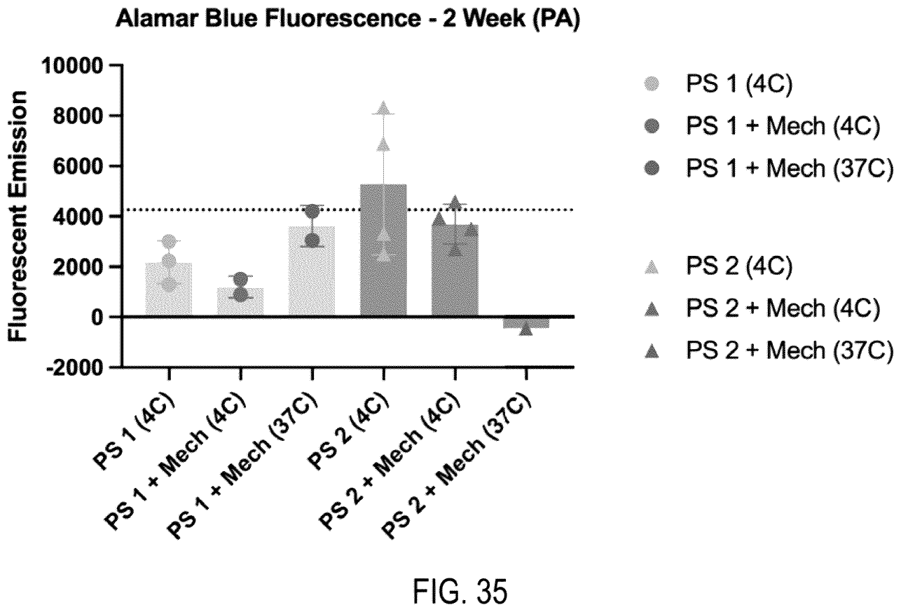

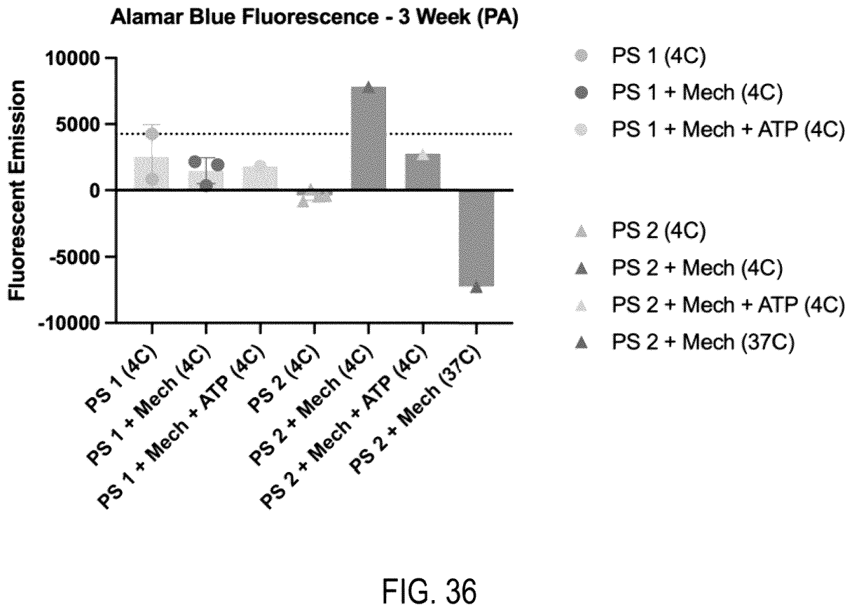

FIG. 34 illustrates the results of PA viability testing after one week of storage. It was found that the viability of pulmonary artery biopsies is slightly reduced from baseline following 1 week of storage. It is believed that this is due to thicker tissue, less diffusion when compared with the valve leaflets. FIG. 35 illustrates the results of PA viability testing after two weeks of storage. Viability of pulmonary artery biopsies was found to remain below baseline after 2 weeks of storage. FIG. 36 illustrates the results of PA viability testing after three weeks of storage. PA samples demonstrate reduced viability following 3 weeks of storage. It is believed that proposed additives as described hereinabove into the preservation solution will improve viability.

### Example B

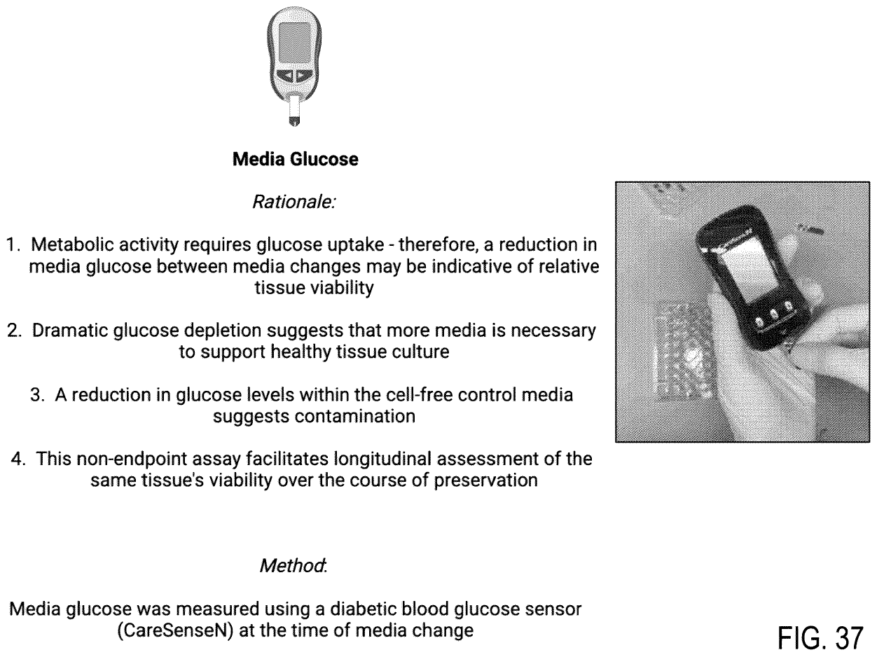

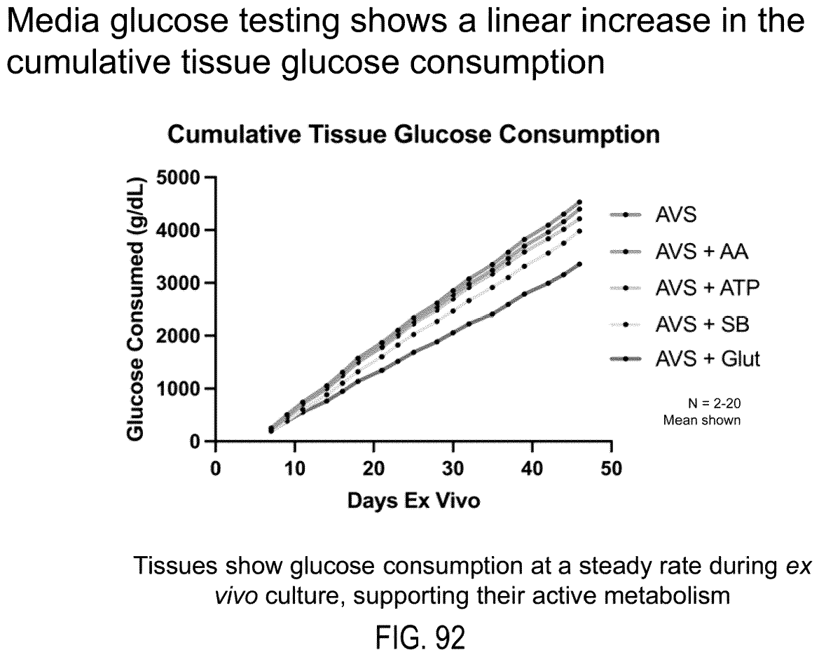

Monitoring tissue glucose uptake between media changes was performed to provide insight as to the metabolic activity of stored valvular tissue, shown in FIG. 37. Without being bound to a particular theory, it is understood that metabolic activity requires glucose uptake and that monitoring tissue glucose uptake between media changes can provide insight as to the metabolic activity of stored valvular tissue. Therefore, a reduction in media glucose between media changes is believed to be indicative of relative tissue viability. Dramatic glucose depletion suggests that more media is necessary to support healthy tissue culture. A reduction is glucose level with the cell-free control media suggests contamination. This non-endpoint assay facilitates longitudinal assessment of the same tissue's viability over the course of preservation. In this example, media glucose was measured using a diabetic blood glucose sensor (CareSenseN) at the time of media change. Results shown in FIG. 92 demonstrate that tissues show glucose consumption at a steady rate during ex vivo culture, supporting their active metabolism.

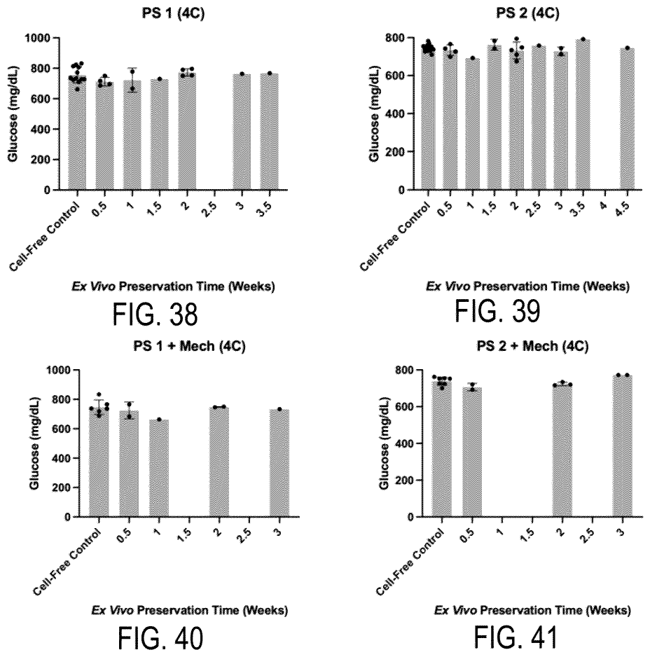

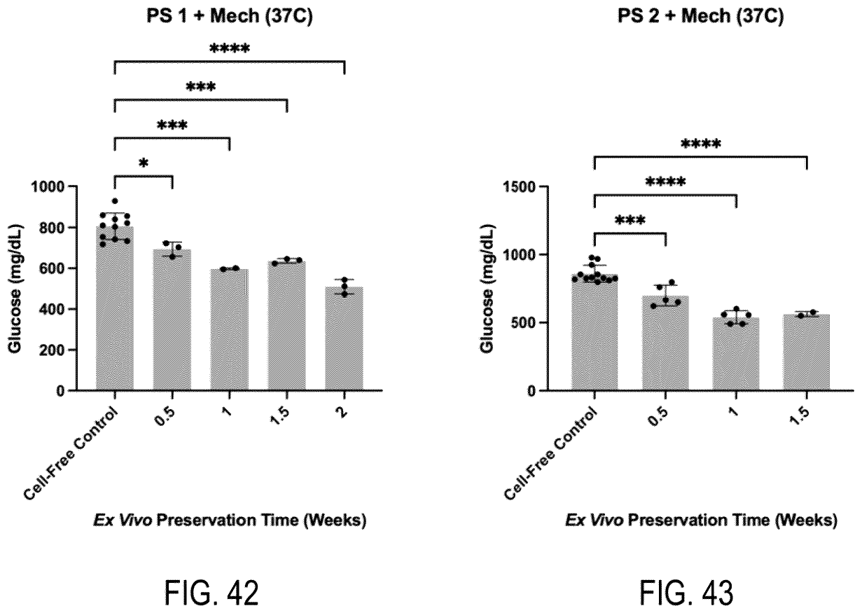

FIGS. 38-39 illustrate culture media glucose levels for fridge-stored valve leaflets using PS1 and PS2, respectively. Culture media glucose levels were found to remain constant over time in fridge-stored samples, indicating that tissues are not taking-up glucose. This may be suggestive of paused or greatly reduced tissue metabolism. FIGS. 40-41 illustrate culture media glucose levels for fridge-stored valve leaflets using PS1 and PS2 with mechanical stimulation, respectively. A lack of tissue glucose uptake is similarly seen in fridge-stored valves under mechanical stimulation. FIGS. 42-43 illustrate culture media glucose levels for incubator-stored valve leaflets using PS1 and PS2 with mechanical stimulation, respectively. In incubated samples, mechanical stimulation is associated with lower media glucose values over time, indicating increased tissue glucose uptake with increased ex vivo preservation time. This may be associated with improved tissue viability.

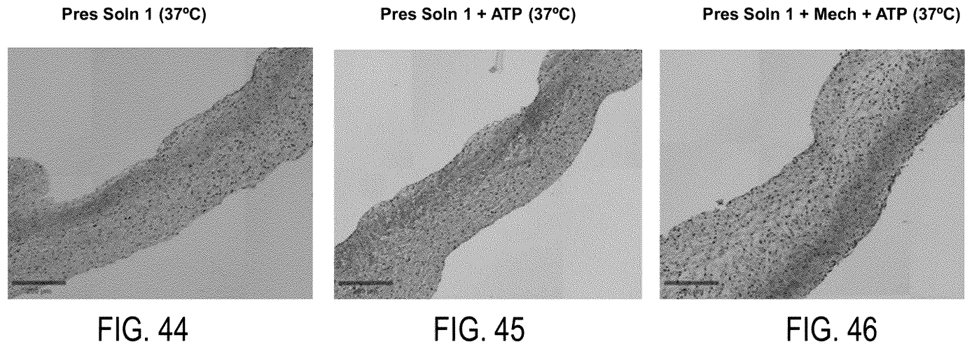

FIGS. 44-46 illustrate porcine leaflet architecture after one-week storage for PS1 at 37° C., PS1 with ATP at 37° C., and PS1 with mechanical stimulation and ATP at 37° C. This data suggests that the leaflet's characteristic trilayered architecture can be preserved through 1 week of the preliminary storage protocol.

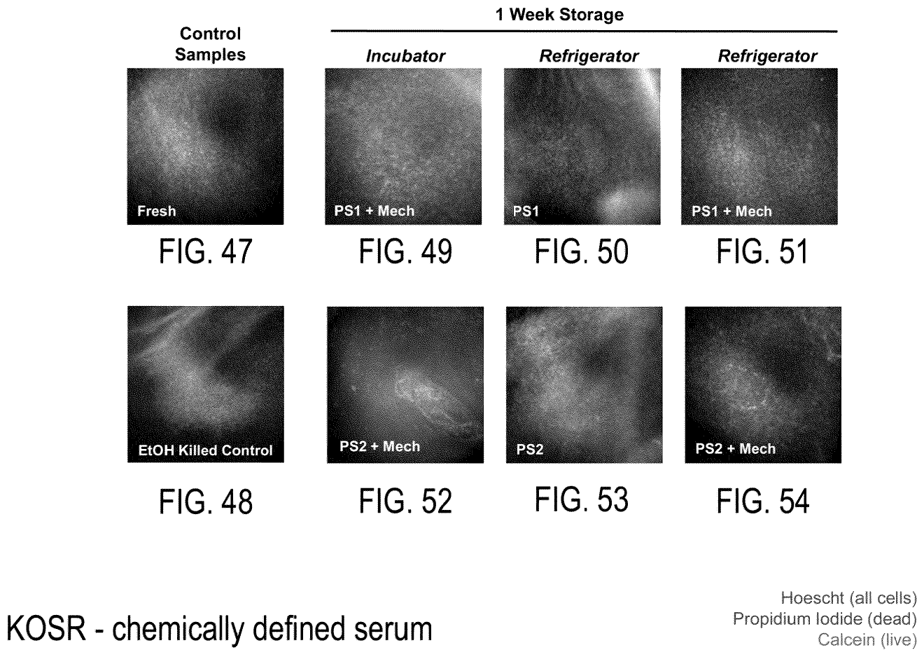

FIGS. 47-54 illustrate imaging tests (Hoechst, Propidium Iodide and Calcein) to show the presence of live and dead cells in porcine pulmonary valve leaflets after one week of storage. Control samples included fresh tissue (FIG. 47) and EtOH killed control tissue (FIG. 48). Imaging was performed on samples with PS1 with mechanical stimulation and 37° C. (incubator) storage (FIG. 49), PS1 and 4° C. (refrigerator) storage (FIG. 50) and PS1 with mechanical stimulation and 4° C. (refrigerator) storage (FIG. 51). Imaging was performed on samples with PS2 with mechanical stimulation and 37° C. (incubator) storage (FIG. 52), PS2 and 4° C. (refrigerator) storage (FIG. 53) and PS2 with mechanical stimulation and 4° C. (refrigerator) storage (FIG. 54). All preservation conditions show presence of viable cells on the leaflet surface after 1 week of storage.

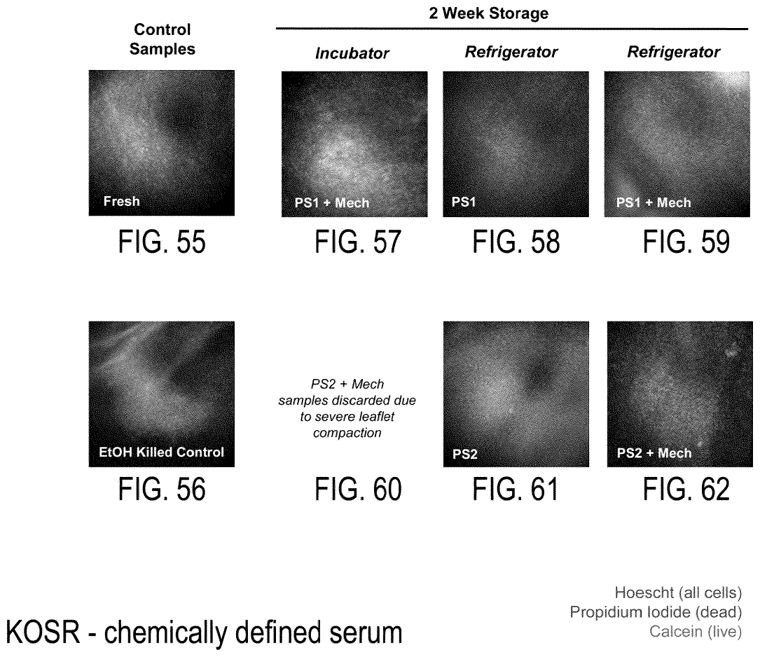

FIGS. 55-62 illustrate fluorescence images of porcine pulmonary valve leaflets after two weeks of storage. Hoechst (all cells), Propidium Iodide (dead cells) and Calcein (live cells) dyes were to show the presence of live and dead cells. Control samples included fresh tissue (FIG. 55) and EtOH killed control tissue (FIG. 56). Imaging was performed PS1 with mechanical stimulation and 37° C. (incubator) storage (FIG. 57), PS1 and 4° C. (refrigerator) storage (FIG. 58) and PS1 with mechanical stimulation and 4° C. (refrigerator) storage (FIG. 59). Imaging was performed PS2 with mechanical stimulation and 37° C. (incubator) storage (FIG. 60), PS2 and 4° C. (refrigerator) storage (FIG. 61) and PS2 with mechanical stimulation and 4° C. (refrigerator) storage (FIG. 62). Live/dead imaging of the leaflet surface demonstrated that mechanical stimulation at 4° C. is associated with increased cell death.

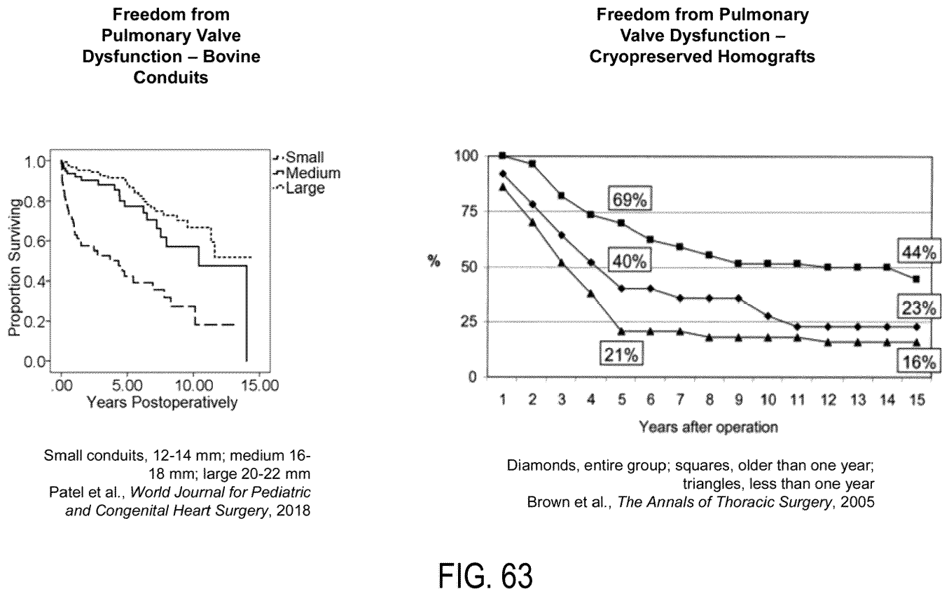

As shown in FIG. 63, common risk factors for valve dysfunction and failure include smaller valve/conduit size, younger age at time of surgery, and complexity of congenital heart disease. Common failure modes include calcification and fibrosis. Younger patients receiving smaller grafts are at highest risk of reintervention or reoperation following a tissue-based valve replacement. Survival results for pediatric patients are shown as freedom from pulmonary valve dysfunction in recipients of bovine conduits (left graph) and cryopreserved homograft (right panel).

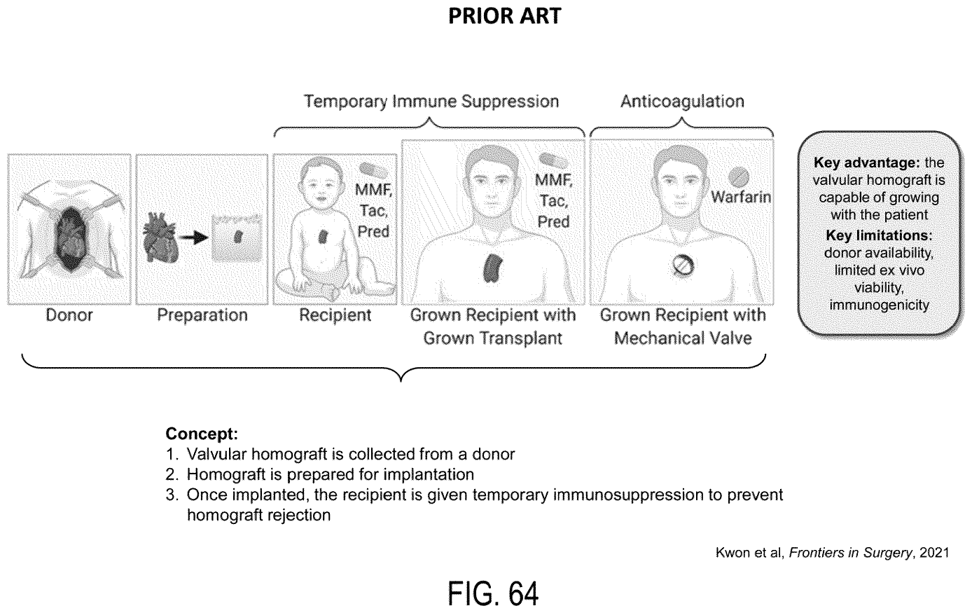

As shown in FIG. 64, recent work highlights “partial heart transplants” as a means of offering living valvular homograft. A key advantage of homografts is that the valvular homograft is capable of growing with the patient. However, key limitations include limited donor availability, limited ex vivo viability, and immunogenicity. Without withing to be bound to theory, the key concept is that: valvular homograft is collected from a donor, and the homograft is prepared for implantation. Once implanted, the recipient is given temporary immunosuppression to prevent homograft rejection.

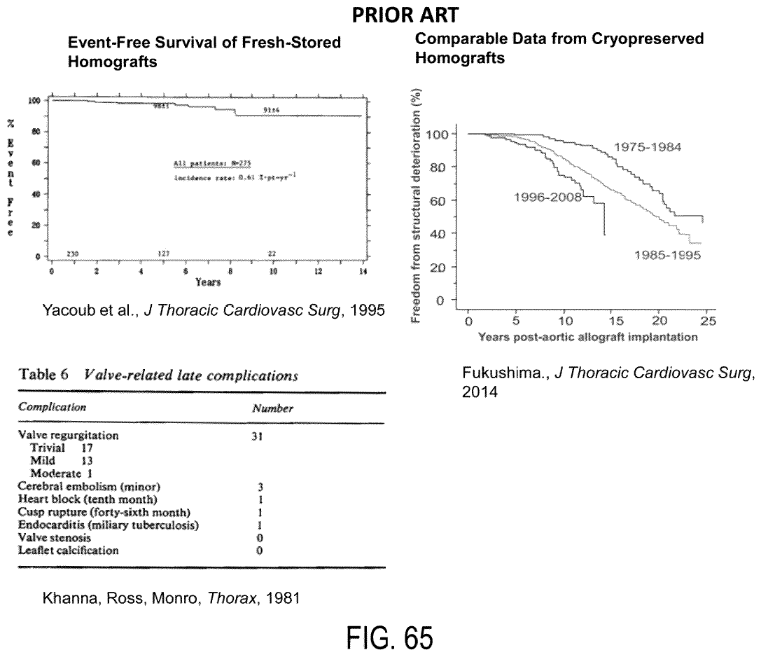

FIG. 65 provides illustrative indications of “fresh” homograft performance. Results indicate that “fresh” homografts have demonstrated freedom from structural deterioration matching or surpassing that of cryopreserved homografts.

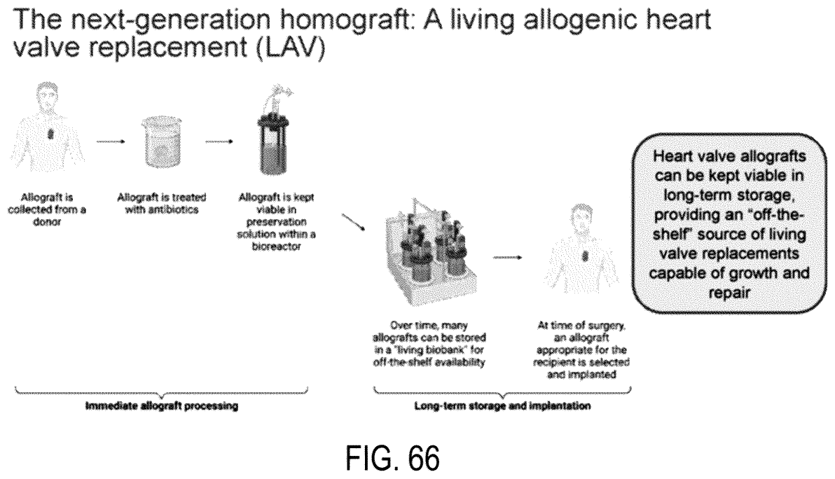

FIG. 66 provides exemplary depictions of a living allogenic heart valve replacement.

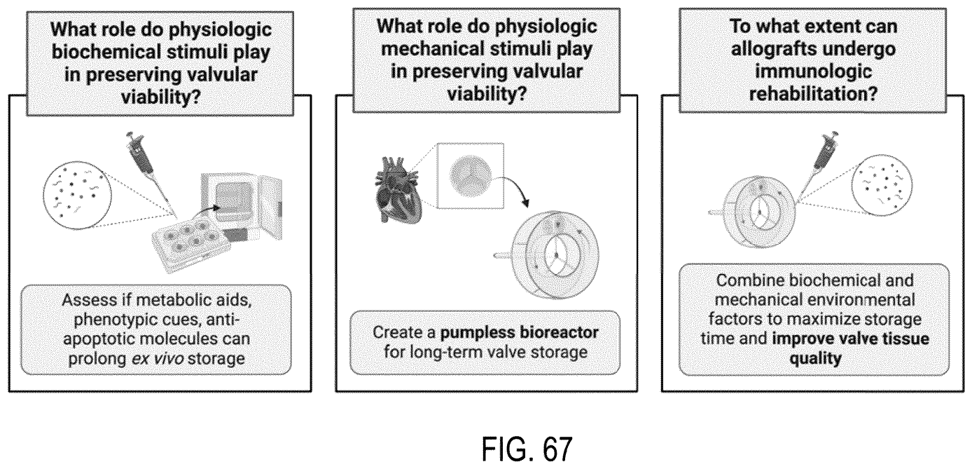

FIG. 67 provides considerations related to preservation of valve allograft tissue physiology. Without wishing to be bound to theory, it was hypothesized that environmental control of key factors contributing to valve degradation, combined with biomimetic mechanical cues, maintains valve physiology ex vivo.

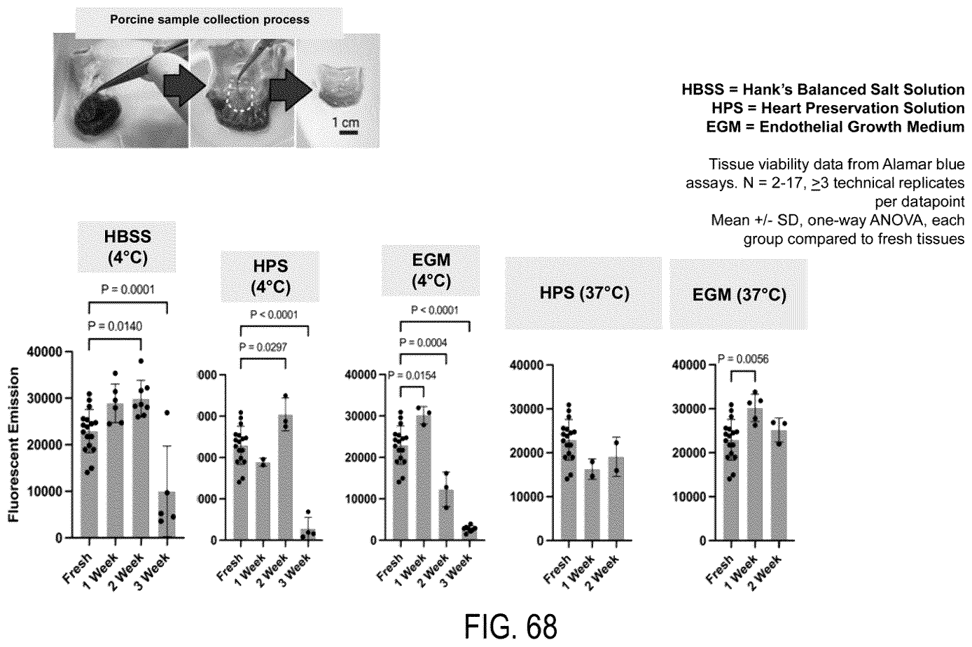

FIG. 68 provides illustrative information concerning porcine pulmonary allograft collection and viability testing. Results suggest living valvular tissue can be preserved for up to 2 weeks ex vivo under the conditions evaluated in FIG. 68.

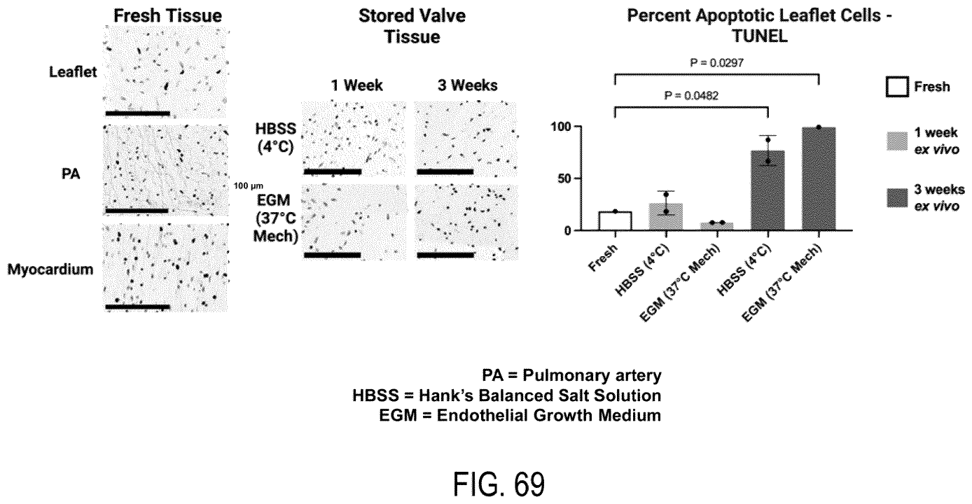

FIG. 69 provides an exemplary TUNEL analysis of valve leaflet viability.

The TUNEL staining results are consistent with Alamar blue analysis of valvular viability.

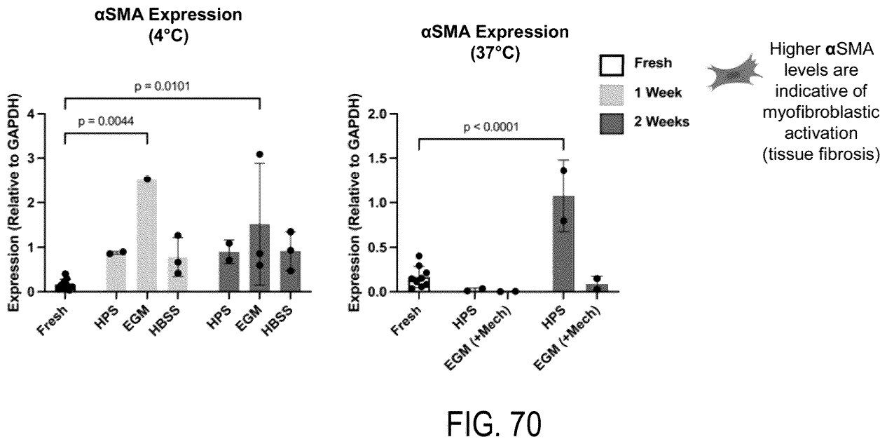

FIG. 70 provides an exemplary phenotypic analysis of resident valve leaflet cells. Smooth muscle α-actin (αSMA) expression levels were evaluated in tissue stored at hypothermic conditions (4° C.) and at normothermic conditions (37° C.). Higher αSMA levels are indicative of activation of myofibroblasts. Results indicate that αSMA expression levels associated with “fresh” tissue can be preserved when the tissue is stored normothermic temperatures and in the presence of EGM (endothelial growth medium).

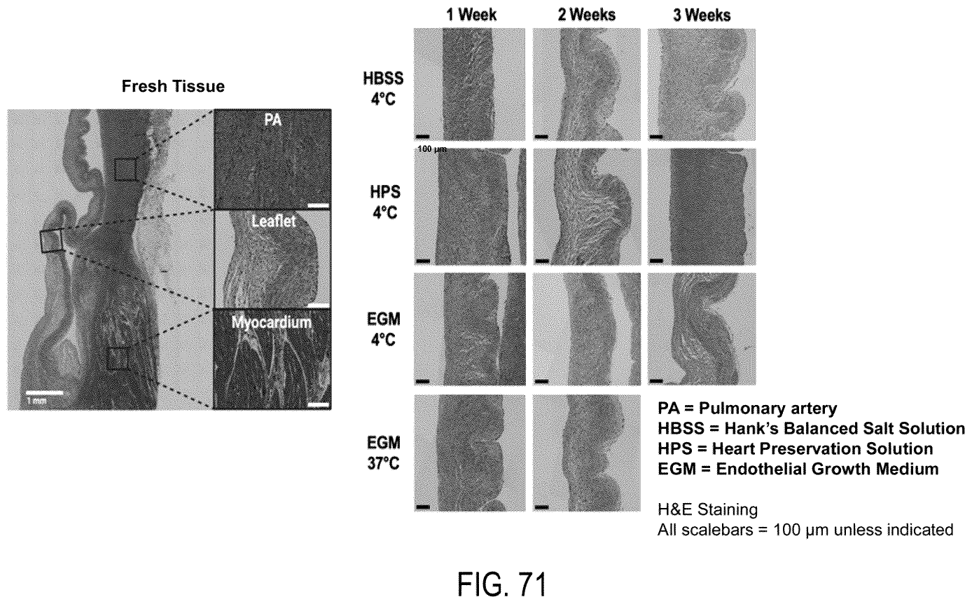

FIG. 71 provides exemplary histologic assessments of valve leaflet architecture preservation. Based on this histological assessment, developing a stratified scoring strategy for valve leaflet histology was determined to be the next step.

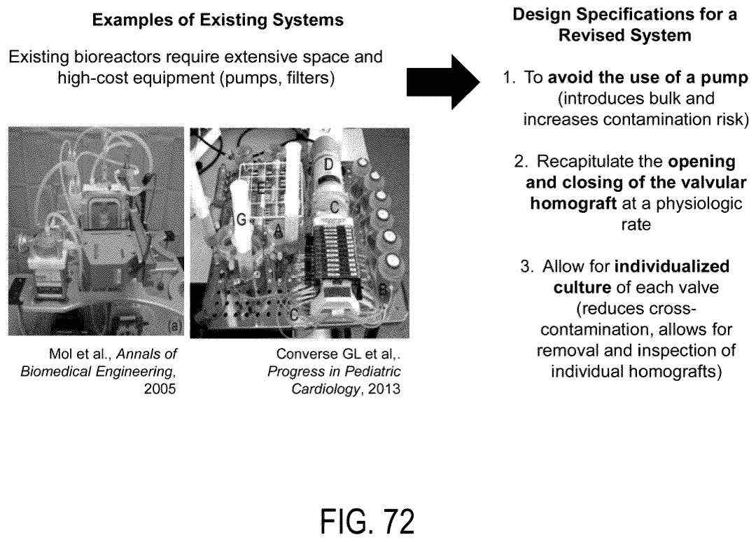

FIG. 72 provides background information concerning creation of a biomimetic mechanical environment for long-term storage. In evaluating existing bioreactors, a key design criterion for the biomimetic mechanical environment was identified as creating a simplified, pump-less bioreactor recapitulating the fluid-dynamic conditions required to open and close the valve. The design specifications for a revised system included: avoiding the use of a pump (introduces bulk and increases contamination risk): recapitulating the opening and closing of the valvular homograft at a physiologic rate; and allowing for individualized culture of each valve.

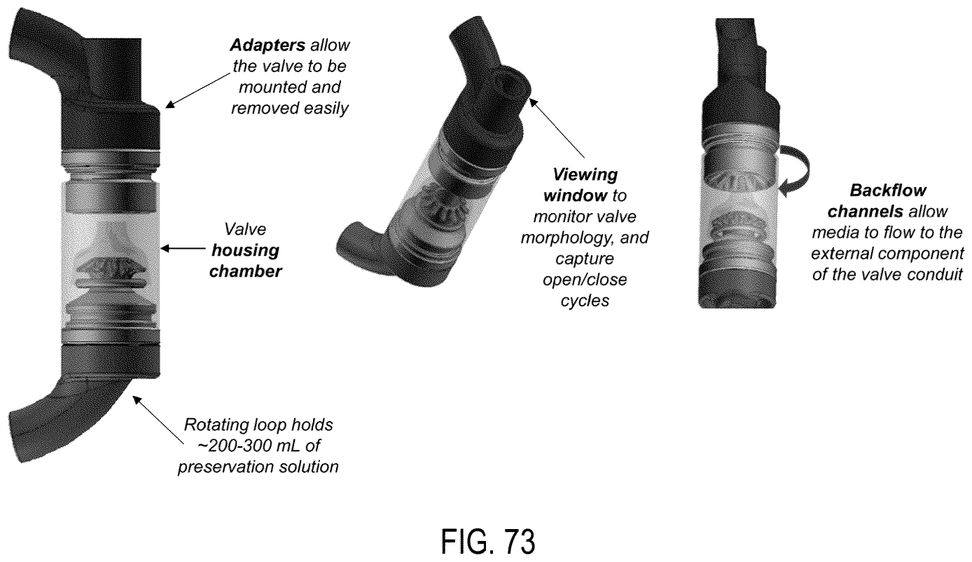

FIG. 73 provides a depiction of an exemplary bioreactor according to the present disclosure. The bioreactor does not require the use of a pump to circulate a composition contained within the bioreactor loop. Furthermore, by inducing open/close cycles at a physiologic rate, the rotating loop-bioreactor preserves a key structure-function relationship for valvular tissue. In some embodiments, the tissue chamber has a diameter in the range of from about 1 cm to about 10 cm. In some embodiments, the tissue chamber has a diameter of from about 2 cm to about 3 cm, from about 3 cm to about 4 cm, from about 4 cm to about 5 cm, from about 5 cm to about 6 cm, from about 6 cm to about 7 cm, from about 7 cm to about 8 cm, from about 8 cm to about 9 cm, from about 9 cm to about 10 cm, including any and all increments therebetween.

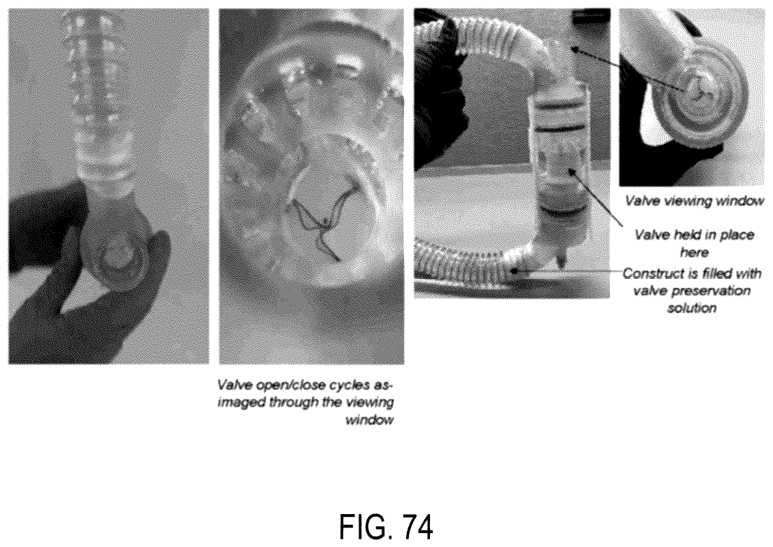

FIG. 74 provides an illustration of an embodiment of a bioreactor according to the present disclosure. The bioreactor can induce valvular open/close cycles without a pump.

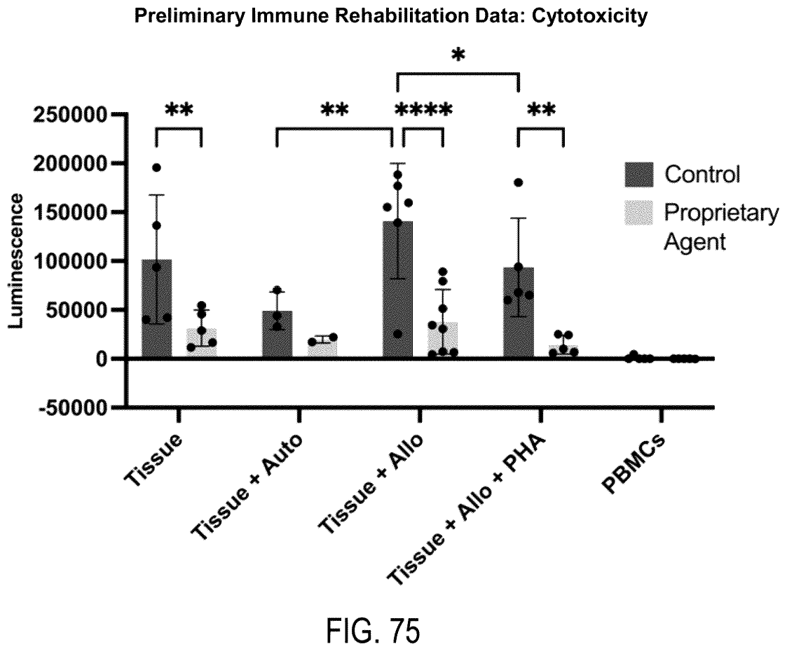

FIG. 75 provides information concerning further considerations related to living valvular tissue for rehabilitating and reducing the intrinsic immunogenicity of living valvular tissue. Without wishing to be bound to theory, the general concept is to reduce homograft ischemic injury, and reduce homograft immunogenicity through exposure to bioactive agents during extended storage. In order to achieve these goals, parameter-controlled analysis of a specific bioactive agent was performed using rat-derived valvular homografts. Results, shown in FIG. 75 demonstrate that incubation with the proprietary agent, (e.g., an anti-inflammatory cytokine) is associated with reduced release of cytotoxic marker LDH as compared to control conditions.

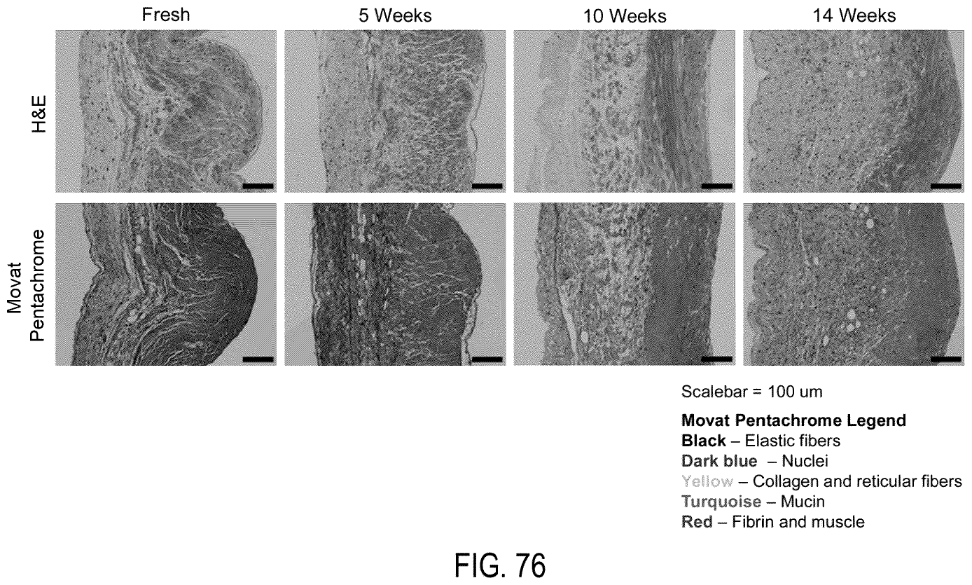

As shown in FIG. 76, aortic leaflet histological sections stained with both H&E and Movat pentachrome stains demonstrate significant microarchitectural shifts over the course of weeks-long storage in electrolyte solution (commercially-available Hanks Balanced Salt Solution). This emphasizes the need for the novel solution herein described. Sections obtained from fresh tissue, or tissue stored for 5 weeks, 10 weeks, or 14 weeks were stained.

### Example C

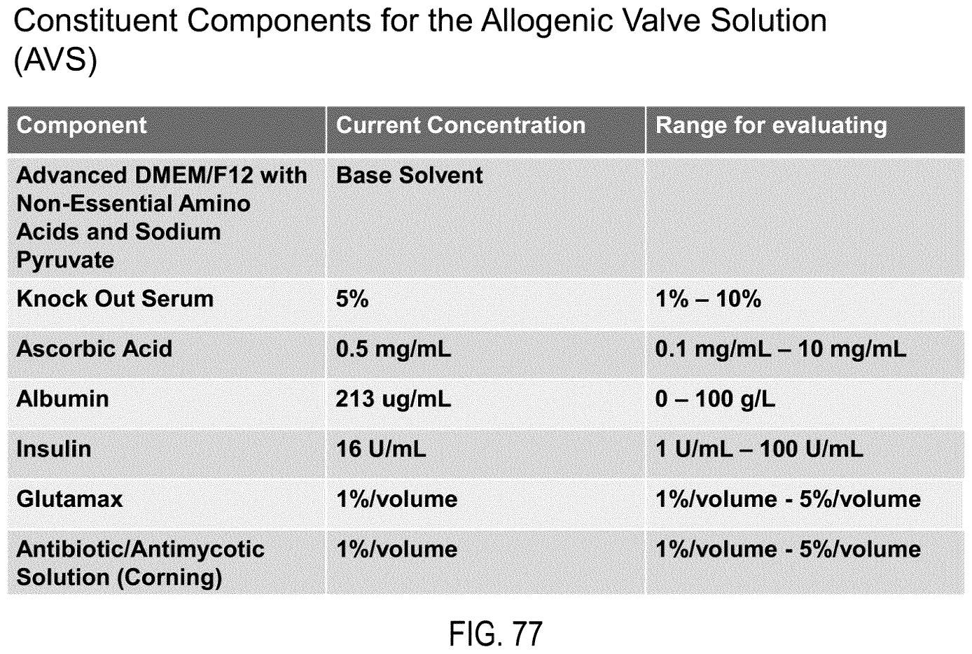

Another culture solution was also prepared for optimizing stored tissue viability, referred to herein as Allogenic Valve Solution (AVS). The components of the AVS are shown in FIG. 77 and include Advanced DMEM/F12 with Non-Essential Amino Acids and Sodium Pyruvate as a basal media. The AVS includes knock-out serum, ascorbic acid, albumin, insulin, 1-glutamine, and an antibiotic/antimycotic solution. The AVS includes knockout serum prepared at 5%. Other suitable amounts of knock-out serum (GIBCO-THERMOFISHER) can include amounts in the range of from about 1% to about 10%. The AVS includes ascorbic acid at a concentration of 0.5 mg/mL. Other suitable concentrations of ascorbic acid can include 0.1 mg/mL-10 mg/mL. The AVS can include albumin at a concentration of 213 μg/mL. Other suitable concentration of albumin can include 0-100 g/L. The AVS can include insulin at a concentration of 16 U/L. Other suitable concentrations of insulin can include 1 U/L-100 U/L, including all intermediate ranges and sub-ranges. The AVS includes 1-glutamine (GlutaMAX, GIBCO-THERMOFISHER) at a concentration of 1%/volume. Other suitable concentrations of 1-glutamine can include 1%/volume-5%/volume. The AVS includes an antibiotic/antimycotic solution (CORNING) at a concentration of 1%/volume. Other suitable concentrations of antibiotic/antimycotic solution can include 1%/volume-5%/volume.

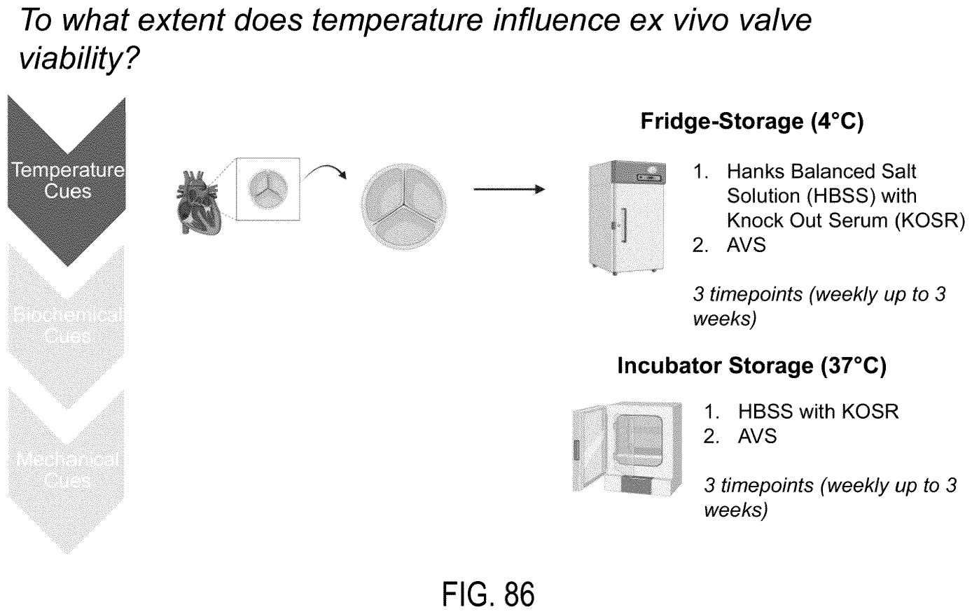

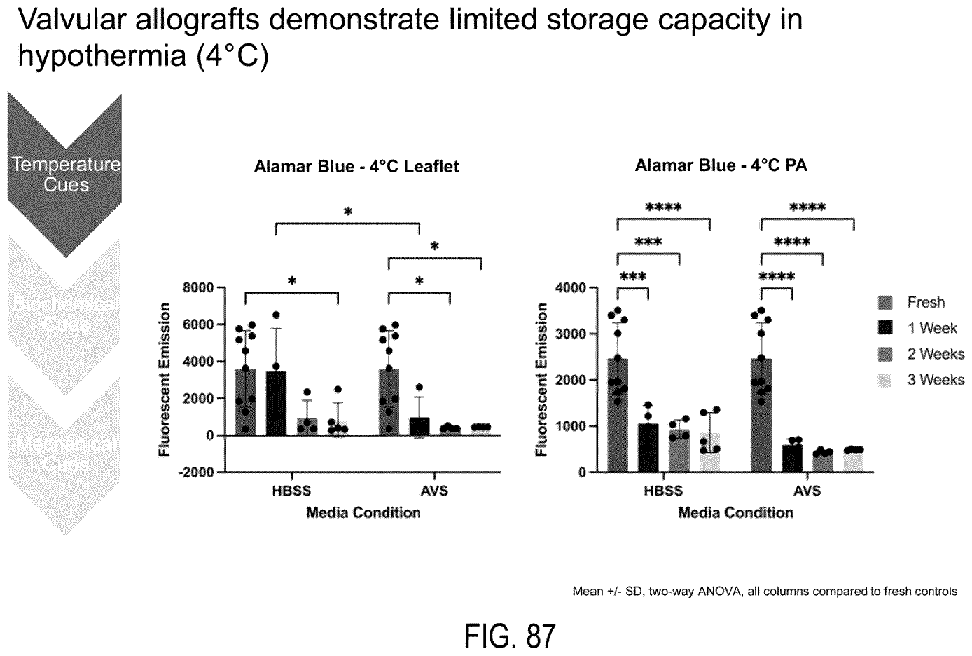

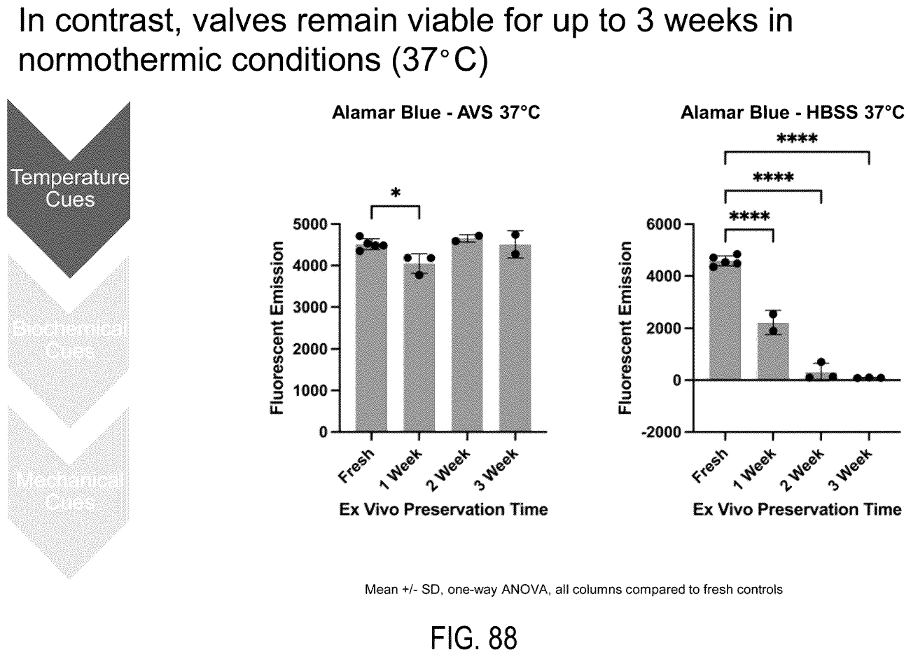

The role of temperature on tissue viability using AVS solution was also evaluated. Hank's Balances Salt Solutions (HBSS) was used as a media control. Tissue was stored under hypothermic (4° C.) conditions or normothermic (37° C.) conditions and evaluated weekly for up to 3 weeks, as outlined in FIG. 86. Valvular allografts demonstrated limited storage capacity in hypothermia (4° C.), shown in FIG. 87. However, valves remained viable for up to 3 weeks in normothermic conditions (37° C.), shown in FIG. 88.

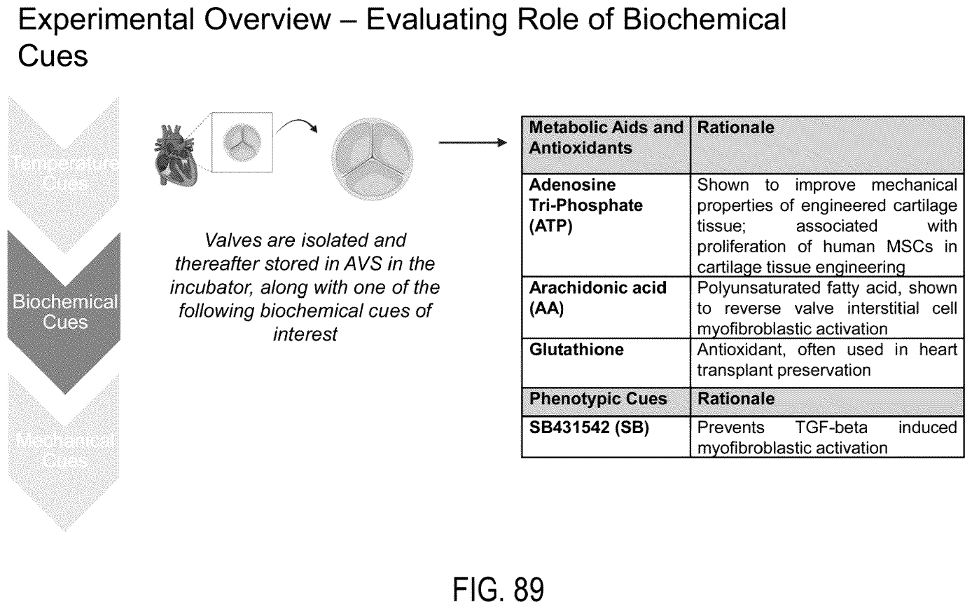

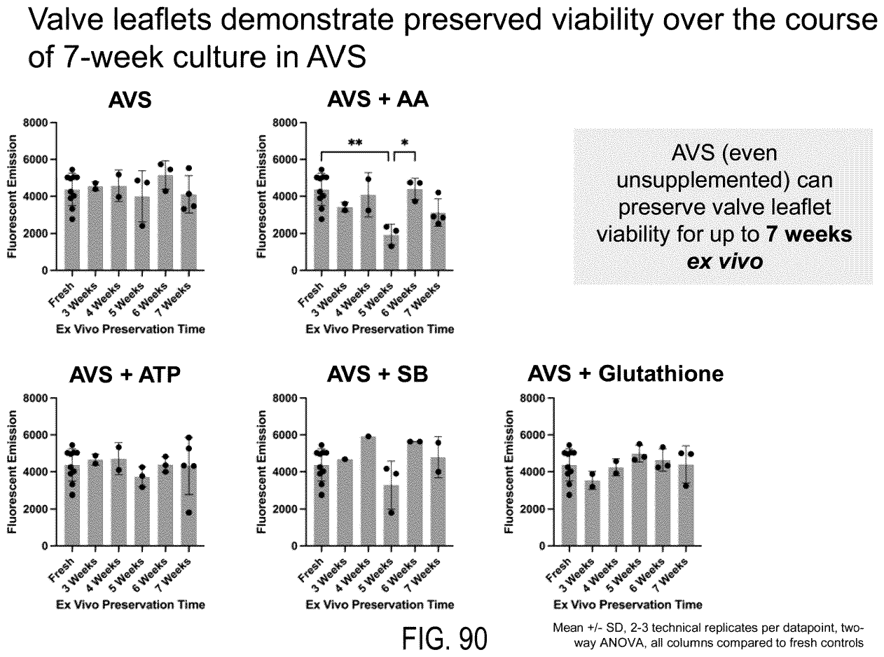

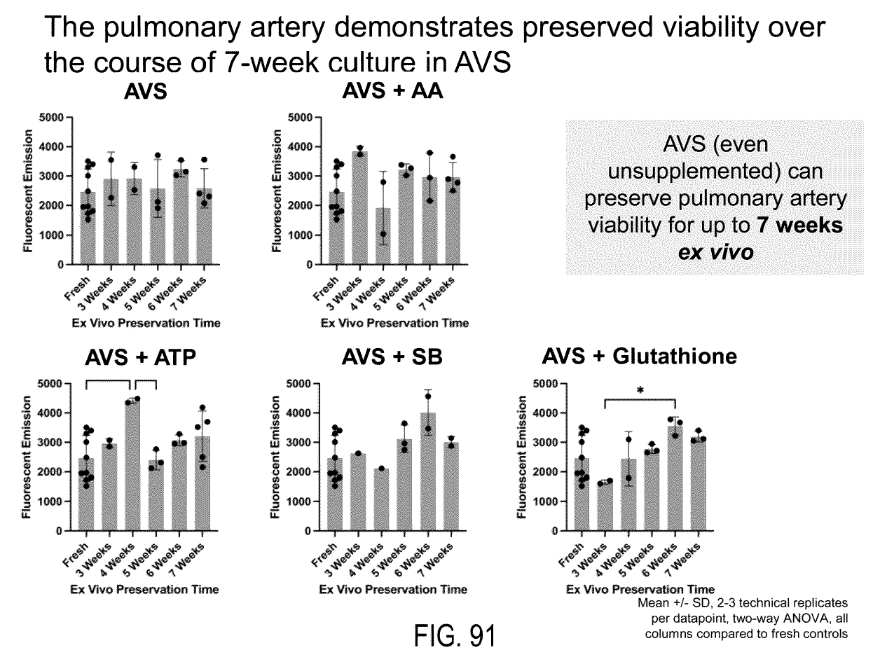

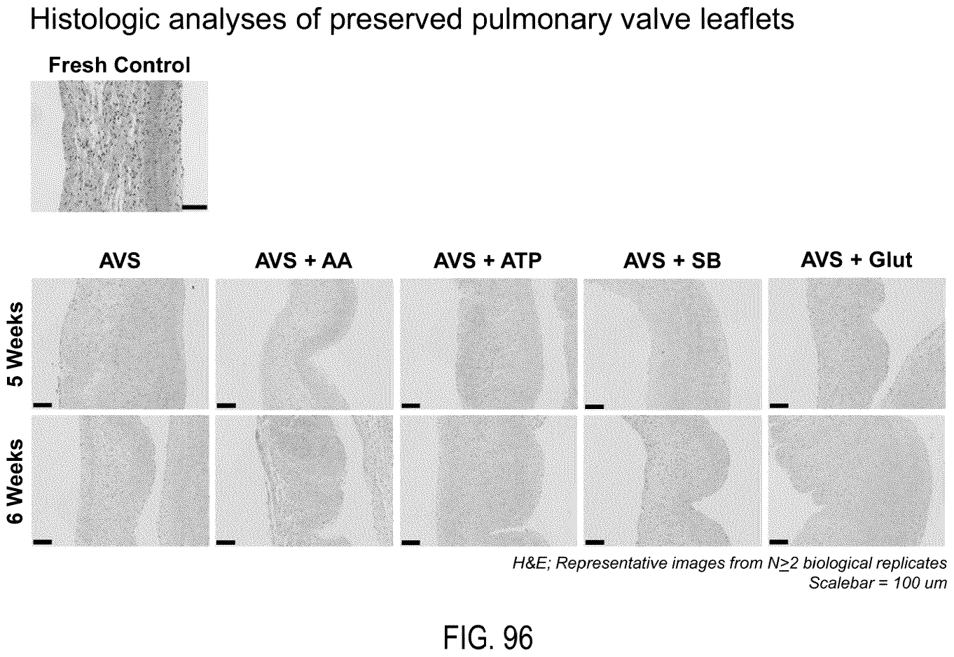

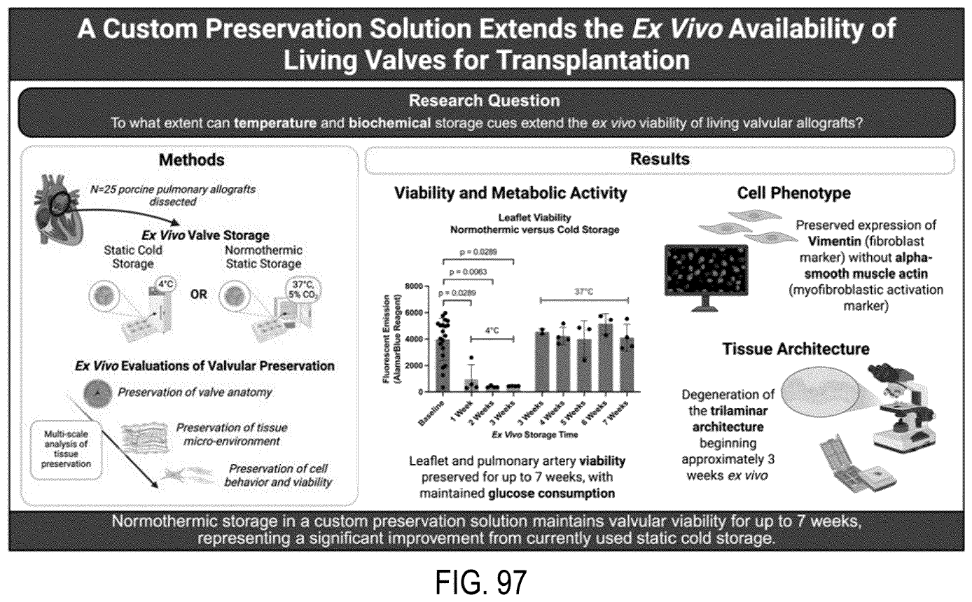

The role of biochemical cues using AVS solution was evaluated. Valve tissues and pulmonary artery tissues were isolated and stored in AVS in normothermia (37° C.). The AVS was supplemented with one of the biochemical cues of interest as outlined in FIG. 89. The biochemical cues included metabolic aids (e.g., ATP, arachidonic acid), antioxidants (e.g., glutathione), and phenotypic cues (SB431542). Results shown in FIGS. 90 and 91 demonstrate that valve leaflets (FIG. 90) and pulmonary arteries (FIG. 91) demonstrate preserved viability over the course of 7-week culture in AVS even without supplemented biochemical cues. Media glucose testing showed a linear increase in the cumulative tissue glucose consumption, demonstrating the stored tissues were metabolically active, shown in FIG. 92. Histological analysis was also performed, shown in FIG. 96, demonstrating viability of tissues stored for 5 weeks or 6 weeks in AVS with or without biochemical supplements.

### Example D

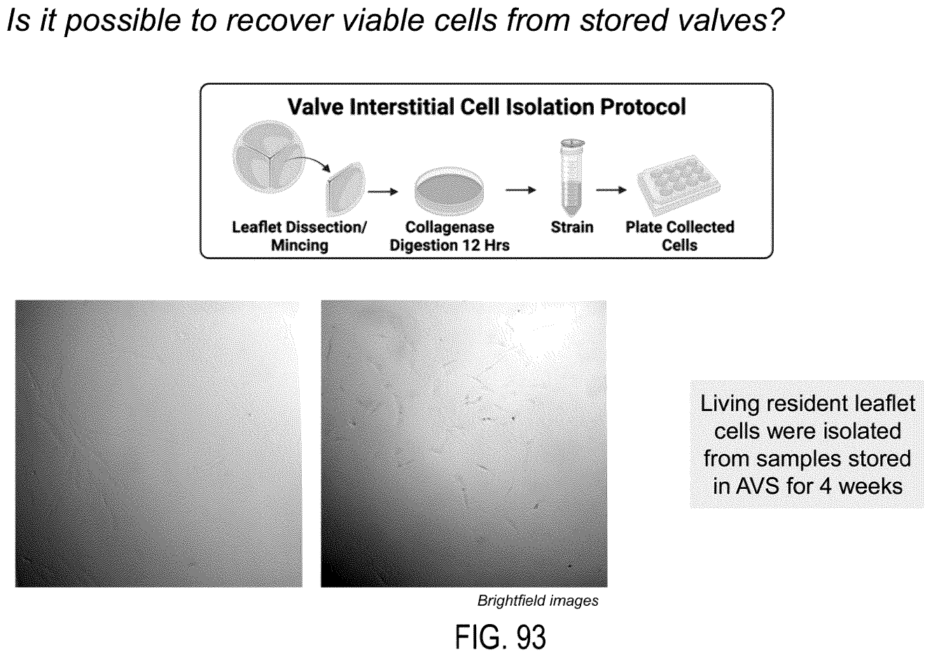

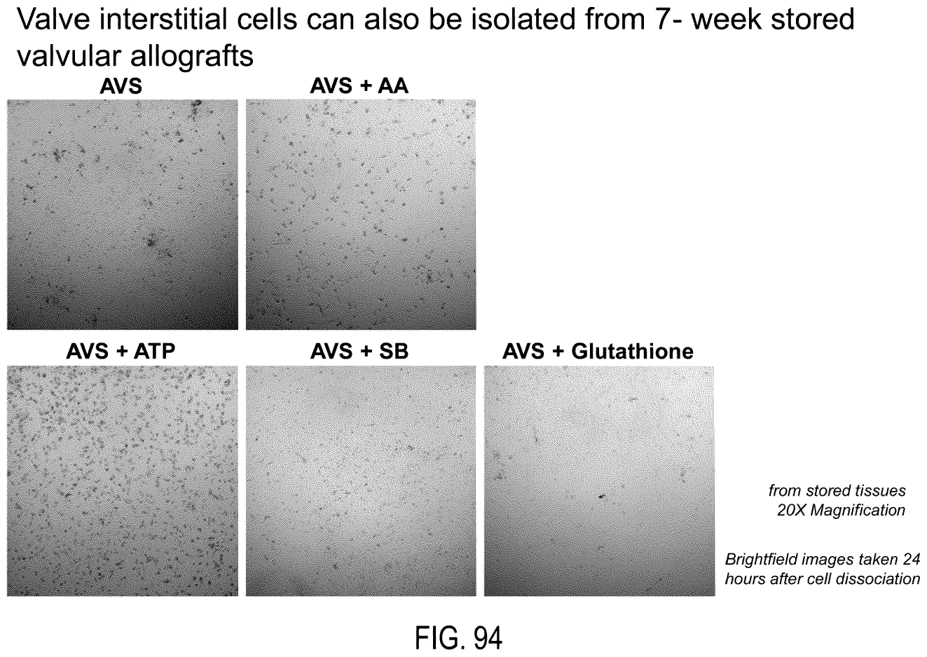

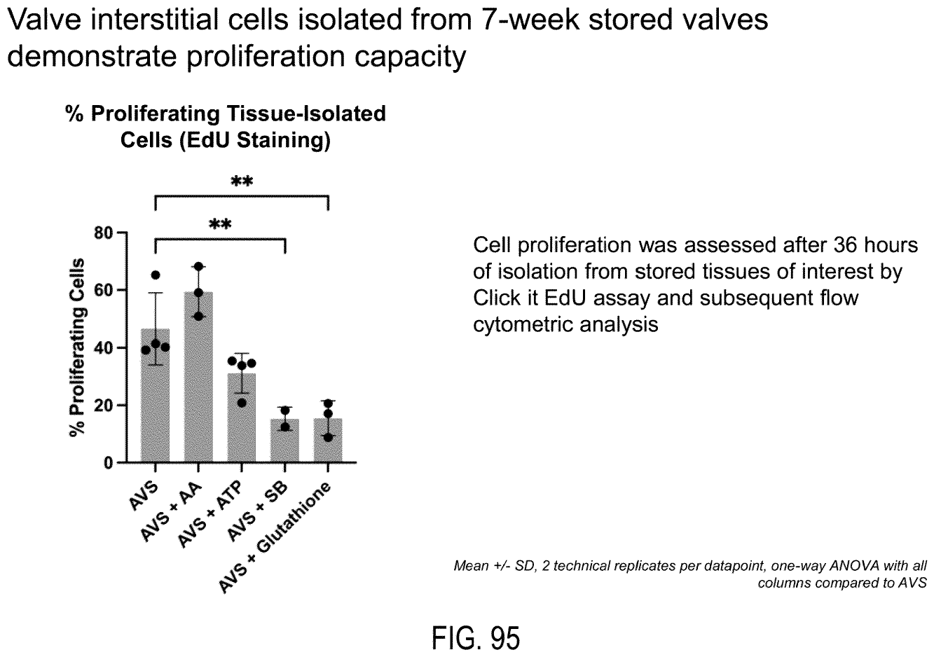

It was next evaluated whether viable cells could be isolated from stored tissue. Stored valve leaflets were dissected and minced. The tissue slurry was digested with collagenase for 12 hours. The digested tissue slurry was then strained to filter out undigested tissue fragments and the supernatants were plates onto tissue culture plastic dishes, as outlined in FIG. 93. Results, shown in FIG. 93 demonstrate that living valve leaflet cells were isolated from tissue samples stored in AVS for 4 weeks. Results shown in FIG. 94 demonstrate that valve interstitial cells can also be isolated from valvular allografts stored for 7 weeks in AVS with or without biochemical supplements. As shown in FIG. 95, cells isolated from valvular allografts stored for 7 weeks demonstrate proliferation capacity. Cell proliferation was assessed after 36 hours of isolation from stored tissues of interest by Click it EdU assay and subsequent flow cytometric analysis.

While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps can be carried out in any desired order (and any desired steps can be added and/or any desired steps can be eliminated).

While heart valve transplantation (HVT) delivers living, growth-capable valves, its clinical implementation is restricted by transplant logistics and donor availability. We developed a preservation solution for the ex vivo storage of living valves that maintains tissue viability for at least 7 weeks.

Porcine pulmonary roots (N=25) were dissected, treated with a clinically used antibiotic cocktail, and stored in a custom-made preservation solution (Valve Preservation Solution, VPS) or Hanks' Balanced Salt Solution (HBSS) with serum for up to 7 weeks. Tissues were preserved in normothermia (37° C., 5% CO₂) and hypothermia (4° C., control group). Tissue preservation was monitored weekly by evaluating viability (AlamarBlue), metabolic activity (media glucose), histologic tissue preservation (H&E, Movat Pentachrome), and valve cell phenotype (immunostaining).

Cold-stored valves (VPS and HBSS) demonstrate significantly reduced viability within 1-2 weeks ex vivo. In contrast, normothermic storage in VPS preserves leaflet viability for 7 weeks, with consistent glucose metabolism. Immunostaining of normothermic VPS-stored valve leaflets shows a quiescent cell phenotype with little expression of alpha-smooth muscle actin or proliferation markers (relative to baseline), and baseline-level leaflet Vimentin expression. Despite preserved cellular viability, leaflet microarchitectural integrity was only maintained for 3 weeks of ex vivo storage.

Normothermic preservation of living valves can support tissue viability for at least 7 weeks ex vivo. With additional preclinical validation, stored living valves could act as a tissue source for HVT, with key advantages in enhanced availability and resource-efficiency.

### Introduction

Despite the global need, there is no heart valve replacement option with long-term durability or growth-capacity. Clinical outcomes underscore the urgent need to develop a cardiac valve replacement capable of growth and remodeling in pediatric patients. Despite their widespread use, cryopreserved human allografts demonstrate failure modes of calcification and fibrosis. Moreover, as many as 75% of the valves implanted in infants fail within 5 years. There is a 40% in-hospital mortality rate following implantation of allograft aortic valves in infants, and an early mortality of 49.4% for recipients of a truncal valve replacement. Primary risk factors for structural degeneration of valvular allografts include young age and small allograft size, with patients often requiring multiple reoperations throughout their lifetime to repair or replace outgrown or structurally degenerated prostheses.

Failure modes associated with cryopreserved and bioprosthetic valves have been attributed to the non-living nature of these tissues. Bioprosthetic valves demonstrate calcific, non-calcific, and fatigue-induced structural deterioration, which is accelerated in children due to interactions between the host serum and implanted biomaterial. Mechanical valves require life-long anticoagulation which increases the recipient's risk of bleeding and thromboembolism, and size limitations inhibit their use in neonates.

Heart valve transplantation (HVT) is a means of delivering a replacement valve with physiologic growth, self-repair, and remodeling mechanisms. The concept underlying HVT is valvular allograft procurement from a donor, followed by semi-emergent implantation of the living valve into the recipient, in a transplant fashion, with immunosuppressive therapy. The presence of living resident valve cells is anticipated to impart long-term durability and growth potential.

The improved performance of living valves underlies the Ross procedure, where pulmonary autografts replace the patient's aortic valve. Pulmonary autografts demonstrate growth and self-repair, with exceptional durability. However, the Ross procedure is unavailable to patients with absent or abnormal pulmonary valves and often requires reintervention due to pulmonary autograft dilation or degeneration of the right ventricular to pulmonary artery conduit used to replace the native pulmonary valve. The growth and durability of valves implanted with orthotopic heart transplants (OHTs) supports the enhanced performance of living valvular grafts as compared to currently available prostheses. In OHTs, the valvular microstructure remains physiologic with preserved interstitial cellularity and valvular endothelium.

Clinical findings have reinforced the encouraging performance of HVTs in pediatric patients, including those only six days old, over several months of follow-up. HVTs exhibit adaptive growth, with longitudinally increasing valvular diameters which positively correlate with increases in the valve recipient's body surface area. Data from multiple centers demonstrate the promise of semilunar HVTs in treating patients with unique congenital heart diseases (CHDs) who present for HVT with heterogeneous primary indications and surgical histories. Clinically, multicenter studies demonstrating HVT's safety and durability are a pre-requisite to more widespread implementation of this technique in the congenital cardiac surgery community.

In parallel, there are important logistic limitations which must be overcome to improve the spatiotemporal availability of HVT: (i) restricted allograft availability, (ii) limited ex vivo viability, and (iii) the cost, time and resource constraints related to minimizing tissue ischemic time. Historically, restricted donor availability and uncertainty about the viability of fresh valvular allografts has led surgeons to adopt cryopreserved allografts.

When a donor valve can be procured, its use is dependent on finding a size-matched recipient, and the time-sensitive nature of fresh tissue transplantation is resource-intensive. These limitations are currently a major barrier to the widespread adoption of this potentially transformative surgical option for children and adults who need a heart valve replacement. There remains a critical need for a preservation strategy that keeps valvular allografts alive, enabling their off-the-shelf availability. We hypothesize that normothermic storage in a custom preservation solution can meet this need by maintaining the viability of living allogeneic valves ex vivo.

### Methods

### Sample Preparation

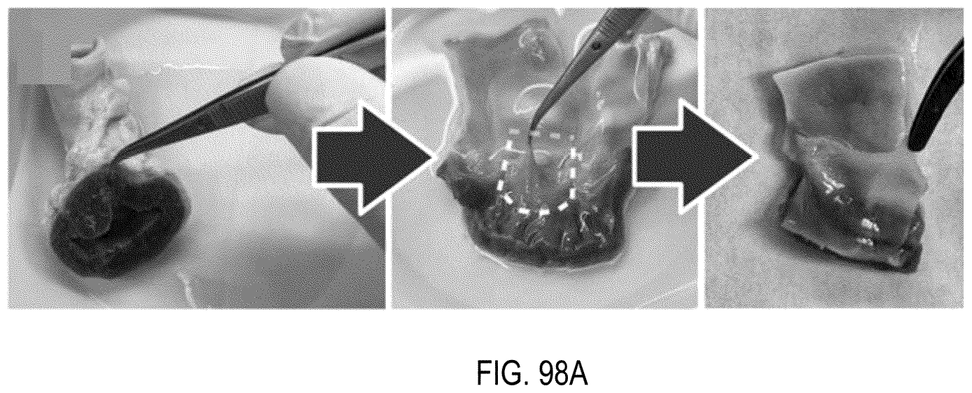

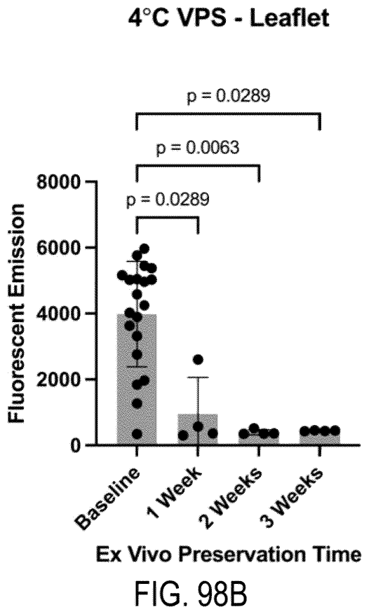

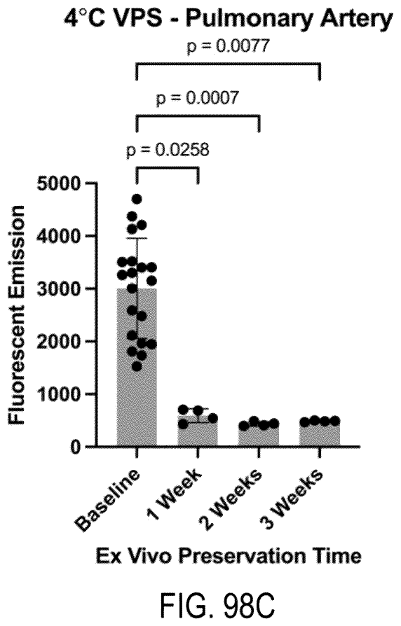

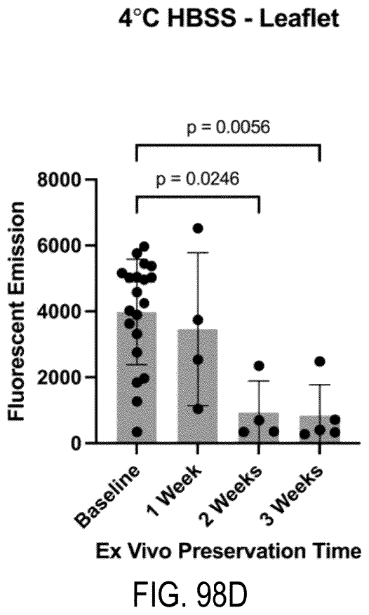

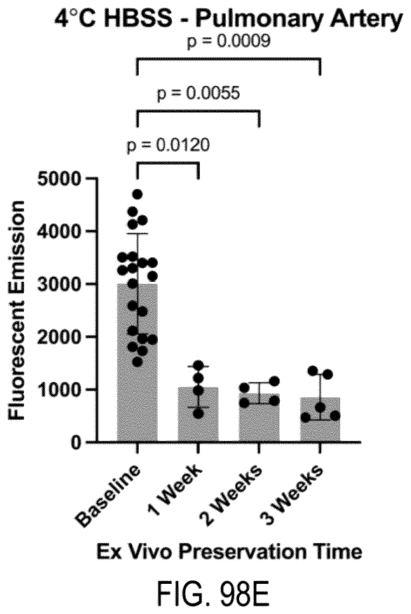

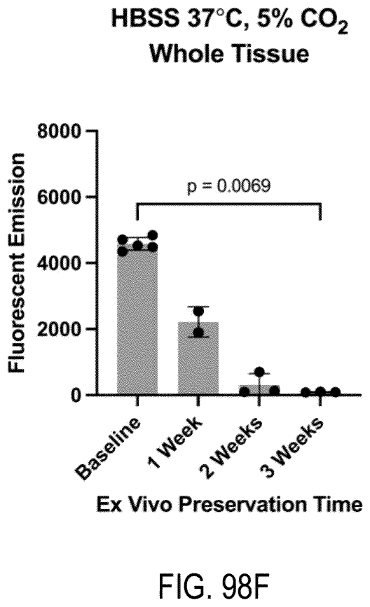

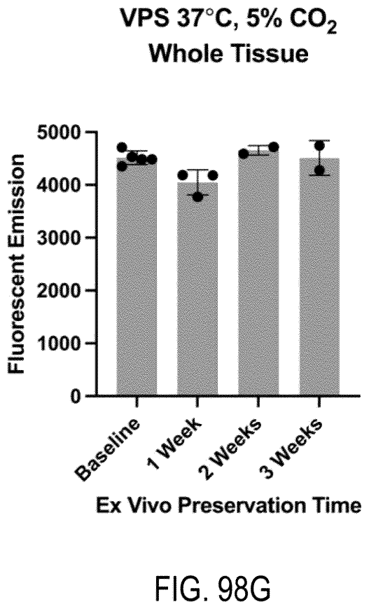

Porcine hearts (N=25, sourced from Animal Biotech Industries: Doylestown, PA) were procured and shipped overnight. On arrival, the pulmonary allografts were harvested. A porcine model was chosen as swine are widely available, to allow for adequate sample sizes and longitudinal testing. Porcine cardiac tissue shares anatomic similarities with human cardiac tissue, and pigs are the preferred large animal model for testing HVT. The pulmonary allograft was selected as it represents the valve most often requiring replacement in children with CHD. Allografts were dissected free of connective tissue and excess myocardium and split into equally sized specimens which included (i) half a valvular leaflet, (ii) a myocardial cuff, and (iii) 1.5 cm of the pulmonary vascular wall (FIG. 98A). Allograft specimens from each heart were evenly and randomly distributed among experimental groups (FIG. 104).

Specimens were treated with antibiotics using a clinically relevant protocol from the European BioBank for valvular allografts consisting of Lincomycin HCl (120 μg/mL: Sigma), Polymyxin B Sulfate (124 μg/mL: ThermoFisher) and Vancomycin (50 μg/mL: ThermoFisher) in nutrient media. Following receipt, samples were incubated for 72 hours in antibiotic solution that was replaced every 24 hours, mimicking the upper limit of static cold storage in clinical practice. Throughout antibiotic treatment, samples were kept on a mechanical rocker at 4° C.

To evaluate the impact of antibiotic treatment on tissue viability, N=3 porcine hearts (Animal Biotech Industries: Doylestown, PA) were procured and shipped overnight. On receipt, samples were incubated on a mechanical rocker at 4° C. in Valve Preservation Solution, with or without antibiotics. Viability testing was performed on receipt (baseline: 24 hours ex vivo), 24 hours post-receipt (48 hours ex vivo), 48 hours post-receipt (72 hours ex vivo), and 72 hours post-receipt (96 hours ex vivo).

### Sample Storage

Two media compositions were evaluated: Hanks Balanced Salt Solution (HBSS) with 5% Knock Out Serum (KOSR: Gibco), and a proprietary Valve Preservation Solution (VPS) developed in-house. VPS contains Advanced DMEM/F12 (Gibco), 5% KOSR (Gibco), 1% GlutaMAX™ Supplement (ThermoFisher), Human Albumin (0.5 mg/mL; Sigma), Ascorbic Acid (213 μg/mL; Sigma), Insulin from bovine pancreas (Sigma), and 1% Antibiotic/Antimycotic (ThermoFisher). Samples were stored at 4° C. (refrigerator) or at 37° C. with 5% CO₂ (cell culture incubator), with media changes every 2-3 days.

For normothermic-stored tissues, a parameter-controlled study was performed to evaluate whether specific metabolic aids could improve the preservation of valvular tissue when added to VPS. The following conditions were evaluated: VPS with Arachidonic Acid (AA: 10 μg/mL, Sigma), VPS with Adenosine 5′-triphosphate (ATP) disodium salt hydrate (60 μM, Sigma), and VPS with reduced L-Glutathione (5 mM, Sigma).

### Viability Testing

Sample viability was tested weekly, in an “endpoint” fashion (tissues exposed to AlamarBlue were not placed back in storage). AlamarBlue testing was performed either in a “whole tissue” fashion, or by separating leaflet and pulmonary artery (PA) tissues. For the former, the entire sample was immersed in 3 mL of solution (90% media, 10% AlamarBlue reagent: ThermoFisher) and incubated for 4 hours on a mechanical rocker, in a cell culture incubator. Following incubation, at least two 100 μL samples of supernatant were transferred to a black-well clear bottom plate (Corning) and fluorescence was measured using an excitation/emission spectrum of 530-560/590 nanometers on a Synergy H2 Hybrid Plate Reader (BioTek). For individual assessment of leaflet and PA tissues, samples of the stored valvular grafts were diced into 2-3 replicate squares (each 3 mm²) and individually incubated in 175 μL of solution for 4 hours on a mechanical rocker, in a cell culture incubator on a 96-well cell culture plate. Following incubation, 100 μL of supernatant was transferred to black-well clear bottom plate (Corning) and read on a plate reader as above.

Glucose consumption was measured via the CareSens® N Blood Glucose Test Strip system. Tissue glucose consumption was calculated by quantifying the difference in glucose concentration between tissue-conditioned media and tissue-free control media at each media change. Tissue-free control media were incubated in the same conditions and for the same amount of time as the tissue-conditioned media. Supernatant lactate and lactate dehydrogenase levels were measured from tissue-conditioned media using the LDH-Glo™ Cytotoxicity Assay (Promega) and Lactate-Glo™ Assay (Promega), performed as per manufacturer's instructions. Luminescence was measured on a Synergy H2 Hybrid Plate Reader (BioTek).

Following 4 weeks of ex vivo storage, valve interstitial cells (VICs) were isolated by mincing valvular leaflets, followed by overnight treatment with 0.2 mg/ml of Collagenase II (Worthington) dissolved in VIC media: Advanced DMEM/F12 (Gibco) with 10% Fetal Bovine Serum (FBS: ThermoFisher), 1% GlutaMAX™ Supplement (2 mMol: ThermoFisher), and 1% antibiotic/antimycotic (ThermoFisher). Samples were incubated in a cell culture incubator on a low-adhesion plate (Corning) agitated on an orbital rocker. Following incubation, the solution was filtered through a 40 μm cell strainer (Corning) and centrifuged at 1200 RPM for 5 minutes. The cell pellet was resuspended in VIC media, with subsequent media changes every 2-3 days. Passages were performed by washing the cell culture well with phosphate buffered saline (PBS), followed by treatment with Trypsin 0.25% with EDTA (ThermoFisher) for 5 minutes in a cell culture incubator. The resultant cell suspension was centrifuged at 1200 RPM for 5 minutes and replated at a ˜1:4 ratio in VIC media.

### Histologic Analysis

Stored valvular tissues were fixed in 4% paraformaldehyde (Sigma) at 4° C. overnight and then transferred to 70% ethanol. Tissues were then paraffin-embedded and sliced into 5 μm sections at the Herbert Irving Comprehensive Cancer Center Molecular Pathology Shared Resource at Columbia University.

For immunofluorescent staining, tissue sections were deparaffinized by sequential treatments with CitriSoly™ (Fisher) and rehydration in serial dilutions of ethanol to distilled water. Heat-induced antigen retrieval was performed using 10 mM sodium citrate buffer (pH 6), and tissue slices were subsequently washed for 5 minutes in washing buffer (0.025% Triton™-X. Sigma, in PBS). Tissues were permeabilized for 20 minutes in 0.25% Triton™-X in PBS and then incubated for 3 hours at room temperature in blocking solution (5% FBS in PBS). Next, sections were incubated in primary antibody solution (5% FBS in PBS) at 4° C. overnight with the following primary antibodies: anti-Vimentin (ab24525, Abcam), anti-alpha smooth muscle actin (NBP1-30894, NovusBio), anti-KI67 (ab15580), Abcam), and anti-von Willebrand Factor (ab11713, Abcam). Tissues underwent 3 10-minute washes in washing buffer, after which they were incubated in secondary antibody (Invitrogen) solution (5% FBS in PBS) for 2 hours at room temperature. Samples once again underwent three 10-minute washes, and were mounted using ProLong™ Diamond Antifade Mountant with DAPI (P36962, Invitrogen) prior to imaging on a 900 Confocal Laser Scanning Microscope (Zeiss) or an ECLIPSE Ti2 Series Confocal Microscope (Nikon).

Immunohistochemical staining was done at the Herbert Irving Comprehensive Cancer Center Molecular Pathology Shared Resource, with tissue sections prepared as above. Sections underwent TUNEL, Hematoxylin & Eosin, and Movat Pentachrome staining. Brightfield scanning was performed on a BX61VS scanner (Olympus). Morphometric analysis of valvular microarchitecture was performed, including evaluation of dead cells on TUNEL and cellularity of the valve leaflet and PA. TUNEL stains were quantified by counting the number of live and dead (positive) stained cells in N=3 evenly distributed regions of interest (ROI) of 0.02 mm² in the leaflet or PA. Counts were averaged to create a single percent of live cells per sample. Cellularity was measured by averaging the number of nuclei at N=3 evenly distributed ROI in the leaflet or PA. The thickness of the leaflet and PA were averaged from H&E cross-sections at N=10 locations. Finally, the relative thickness of extracellular matrix (ECM) components was averaged from measurements at N=5 locations per sample. Morphometric analyses were performed using ImageJ and were blinded, except for measurements of leaflet and PA thickness.

### Statistics

AlamarBlue data, glucose consumption, lactate and lactate dehydrogenase release, and morphometric analyses were plotted and analyzed using GraphPad Prism 10 (GraphPad Software, LLC). Statistical analysis for >2 groups was performed via Kruskal-Wallis test with Dunn's multiple comparisons test, comparing each timepoint to the baseline or first-available value. A p-value of <0.05 was considered statistically significant.

### Results

Valvular leaflets showed preserved viability following antibiotic pre-treatment, whereas the PA tends towards reduced viability following 48 hours of antibiotic treatment (72 hours ex vivo) (FIG. 105). When stored in VPS at 4° C., valvular grafts demonstrated significant loss in leaflet and PA viability within 1 week ex vivo (p=0.029 and p=0.026 versus the initial (baseline) viability: N=4-20, FIGS. 98B-C). Valve leaflets and PA sections showed a near-total loss of viability within 2 weeks ex vivo when stored in HBSS with KOSR at 4° C. (p=0.025 and p=0.0055 versus baseline viability: N=4-20, FIGS. 98D-E). Similarly, when stored in HBSS with KOSR at 37° C., 5% CO₂, valves showed significantly reduced whole tissue viability by three weeks (p=0.0069 versus baseline viability, N=2-5, FIG. 98F). Combining a normothermic environment with VPS uniquely preserved whole tissue viability for 3 weeks (N=2-5, FIG. 98G).

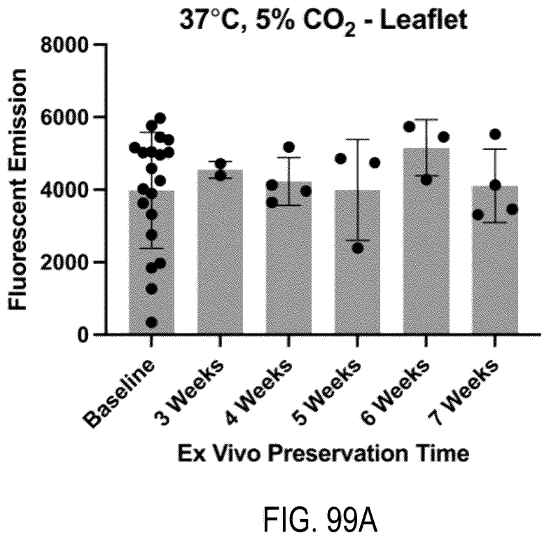

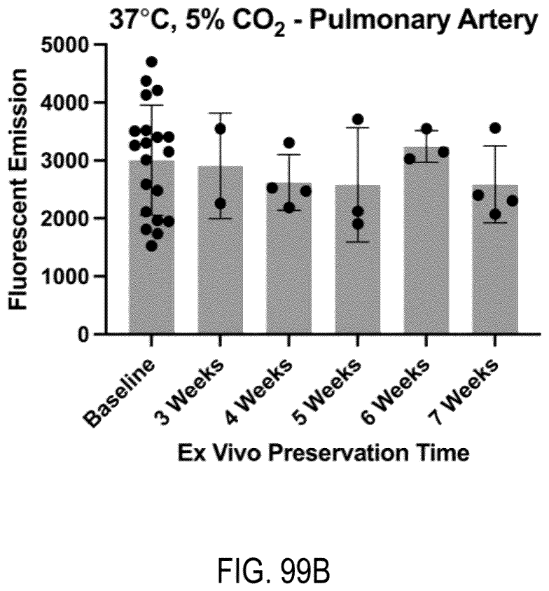

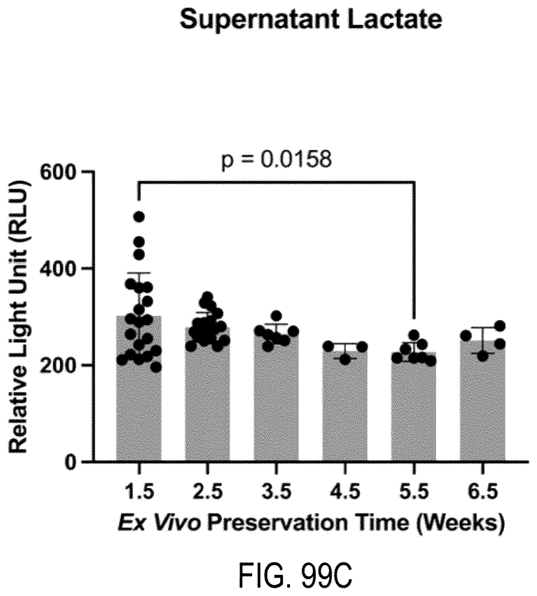

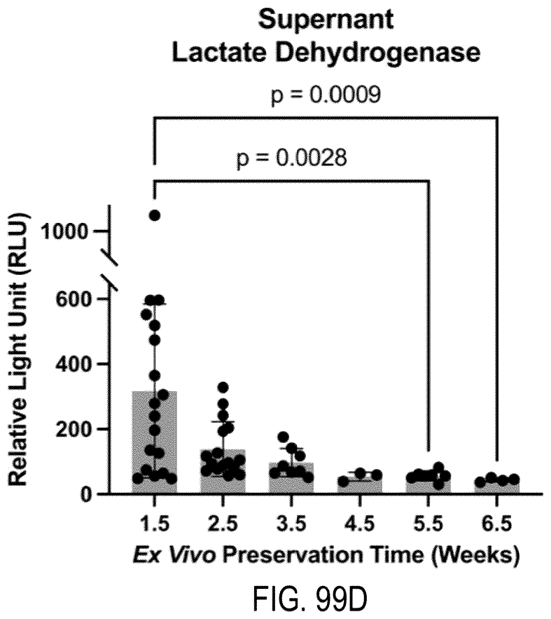

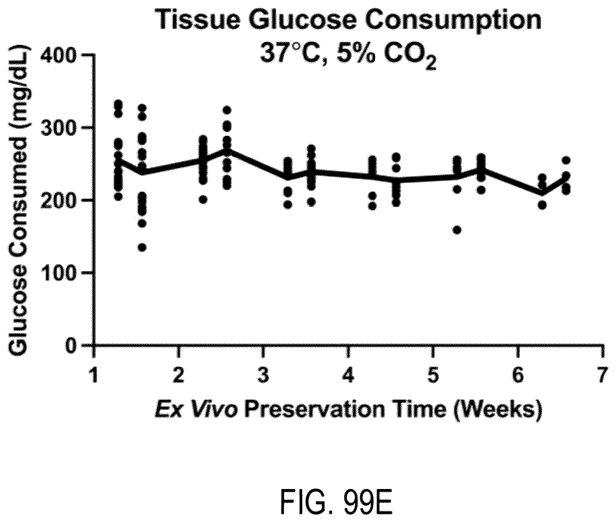

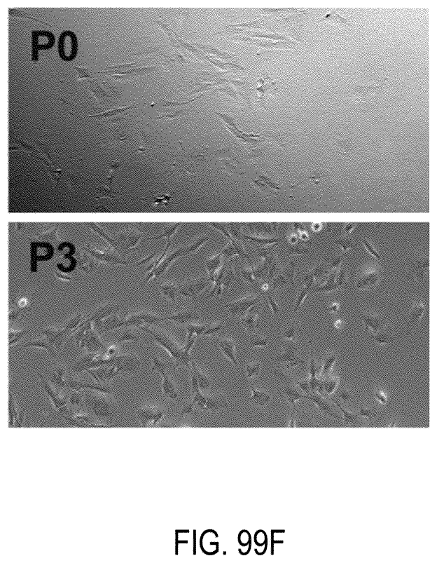

At extended storage timepoints, tissues stored in VPS at 37° C., 5% CO₂ (cell culture incubator) demonstrated preserved viability of the leaflet and PA for up to 7 weeks (no significant differences versus baseline viability, N=2-20; FIGS. 99A-B). Variability in viability between tissues decreased from baseline throughout normothermic storage. Supernatant lactate levels remained relatively constant over time (N=4-20, FIG. 99C). In contrast, the lactate dehydrogenase levels in culture-conditioned VPS significantly decreased within the first weeks of storage (p=0.0028 at 5.5 versus 1.5 weeks ex vivo, N=4-20, FIG. 99D). Glucose consumption-measured as a marker of metabolic activity-was consistent among normothermic-stored valvular tissues and demonstrated decreased variability over time (N=4-20, FIG. 99E). At 4 weeks ex vivo, VICs isolated from valve leaflets that had been stored in VPS in normothermic conditions were viable, proliferative, and could be passaged over 5 times (FIG. 99F).

We next tested whether individual metabolic aids (Adenosine Triphosphate, Arachidonic Acid, and Glutathione) would extend the valve's longitudinal viability. We found no improvement in longitudinal tissue viability in these conditions, suggesting that VPS alone is sufficient to sustain tissue metabolism and protect viability (N=2-20: FIGS. 100A-F). Similarly, no metabolic aids were associated with an increase in glucose consumption over time (N=2-20); FIGS. 100G-I). No major differences were observed in the valvular microarchitecture between these conditions at 5- and 6-weeks ex vivo (N=2-3; FIG. 100J). Therefore, for the remaining studies, we focused on tissues stored in VPS alone.

On Hematoxylin & Eosin (H&E) stain, the valve leaflet's physiologic trilaminar leaflet architecture was appreciable throughout the first 3 weeks ex vivo, with independent fibrosa, spongiosa, and ventricularis layers (N=1-3, FIG. 101A). From 3 to 6 weeks ex vivo, loss of the trilaminar leaflet architecture was observed: by 6 weeks ex vivo, most of the leaflet cross section was composed of collagen (N=1-3: FIG. 101A). Leaflet thickness and cellularity did not change significantly throughout storage (N=1-3, FIG. 101C, 101D). TUNEL staining for DNA fragmentation demonstrated interstitial cell death from baseline to 2 weeks ex vivo, with viability remaining at ˜70% thereafter (N=1-3: FIG. 101B, 101E).

On Movat Pentachrome staining, valvular leaflets stored up to 3 weeks ex vivo demonstrated preserved ECM composition, with physiologic presence of elastin (black) in the ventricularis layer, glycosaminoglycans (blue) in the spongiosa layer, and collagen (yellow) in the fibrosa layer at thicknesses similar to those in the baseline leaflet cross-section (N=2: FIG. 102A). However, by 6 weeks ex vivo, the proportion of the leaflet composed of collagen increased to 58% (from 31% at baseline), with a corresponding decrease in the thickness of the proteoglycan/glycosaminoglycan layer to 16% of the leaflet (from 44% at baseline). The thickness of elastin in the ventricularis position was preserved (N=2: FIG. 102B).

At the level of cell phenotype, VICs demonstrated preserved expression of Vimentin (fibroblast identity marker) throughout storage, without upregulation of α-Smooth Muscle Actin (marker of myofibroblastic activation). VICs did not demonstrate significantly upregulated KI67 expression (proliferative marker) throughout storage (N=2: FIG. 103). The layer of valve endothelial cells surrounding the leaflet remained intact through 2 weeks ex vivo, after which valve endothelial cells were inconsistently observed (N=2, FIG. 103).

Histologic cross-sections of the PA demonstrated preserved thickness and cellularity throughout 6 weeks of ex vivo preservation (N=2-3, FIG. 107D, 107E). TUNEL stains revealed a steadily decreasing percentage of live cells over the course of storage with 70% of cells alive at 3 weeks and 45% of cells alive at 6 weeks (N=1-3, FIG. 107F). Movat Pentachrome staining demonstrated consolidation of elastin fibers throughout the PA cross-section by 3 weeks ex vivo (N=2, FIG. 108).

### Discussion

The recent introduction of HVT into the congenital cardiac armamentarium has revealed the potential of living, growth-capable valves in overcoming the limitations of currently available valve prostheses. While fresh valve transplants are capable of growth, they are not available off-the-shelf. On-demand availability is crucial to providing the right valve to the patient when physiologically indicated, maximizing donor tissue availability and decreasing the costs and resources required for semi-emergent valve transplantations. This is emphasized by the fact that 18% of donor hearts and valves intended for neonates are discarded due to transplant logistics.

Cryopreservation allows for off-the-shelf availability: however, it is correlated with reduced cell viability and pathologic modifications to the valvular ECM. Heart valve tissue engineering relies on the formation of physiologic neo-tissue, which is a highly variable process that can result in adverse remodeling and valve failure.

Here, we demonstrate that static cold storage (previously used for homovital allografts, and standard practice in transplantation) is insufficient to preserve ex vivo valvular tissue viability for more than 1 week ex vivo. This was the case both when storing valvular allografts in HBSS (the electrolyte solution used to store homovital allografts at some institutes), as well as when storing valves in preservation solution. Our observations emphasize the limitations of the currently used methods. They also highlight the importance of considering alternate storage environments (varying temperature, biochemical, and mechanical cues) to improve the ex vivo availability of living valves.

We therefore turned to normothermic storage of valvular allografts. This was motivated by the success of the TransMedics Organ Care System in extending acceptable ex vivo preservation time for heart transplants, and by the promise of novel advances including xenogenic cross-circulation and ex vivo lung perfusion in expanding donor lung availability. These systems represent normothermic environments wherein tissues are perfused and metabolically supported. We developed a custom preservation solution inspired by the following elements: (i) traditional culture medium for VICs, (ii) existing cardiothoracic organ preservation solutions, and (iii) additives to support tissue physiology and metabolism (e.g., ascorbic acid for neutralization of reactive oxygen species, albumin for endothelial protection).

Our results demonstrate, for the first time, that the ex vivo viability of valvular allografts is significantly extended in a normothermic environment. Similarly to normothermic storage strategies for the heart and lungs, our study suggests that the extended viability of valvular tissue is a result of combining physiologic temperature with a nutrient-laden solution capable of promoting tissue homeostasis.

While in normothermic storage, preserved glucose uptake and lactate release, with concomitantly preserved cellularity, cumulatively suggest that tissue metabolic activity was sustained. AlamarBlue viability and glucose consumption showed markedly reduced inter-tissue variability for stored allografts as compared to baseline tissues, supporting our strategy's ability to maintain tissue homeostasis over time. Baseline variability may be the result of early tissue damage induced by transport and dissection. This would be consistent with the high early lactate dehydrogenase release, which rapidly decreased in the first weeks following the transition to normothermic storage.

At 4 weeks ex vivo, VICs derived from ex vivo stored leaflets were viable and remained capable of proliferation. This is promising for in vivo function, as VIC proliferation represents a key homeostatic function required for self-repair and remodeling of injured or growing valves.

Histologic staining further validated AlamarBlue and glucose-level results. TUNEL quantification showed preservation of leaflet viability, with some cell death in the first two weeks ex vivo and maintained viability thereafter. VICs retained Vimentin expression, without evidence of myfibroblastic activation. This is indicative of preserved VIC quiescence—an important point, as leaflet fibrosis and retraction are key failure modes of tissue engineered valves. The loss of valve endothelial cells at 2 weeks ex vivo represents a potential limitation to this strategy. However, it may also contribute positively to in vivo function. Neo-endothelialization of acellular valvular leaflets has been observed in vivo for tissue engineered heart valve replacements. Furthermore, loss of the donor endothelium may reduce the risk of an adverse host immune response, as endothelial cells have been hypothesized to strongly contribute to the immunogenicity of valvular grafts.

PA sections similarly demonstrated preserved thickness and cellularity, without significant alterations to the tissues' architecture. Consolidation of elastin strands was visible by 3 weeks ex vivo: in vivo studies will be required to evaluate the implications on the PA's function. We observed a gradual loss in the percent of live cells within the PA on TUNEL staining, which is consistent with the limitations of oxygen diffusion in the thicker PA tissue. The extent to which this cell death influences graft function will depend on whether adjacent recipient PA cells recellularize the graft in vivo—as is seen in decellularized valvular grafts and tissue engineered heart valves. In addition, recent work from Aykut, Turek, and Overbey et al. highlights that even 50% tissue viability may support preserved valvular growth capacity in vivo. We did not evaluate the preservation of myocardial tissue, as it is not expected to remain viable on reimplantation and is frequently trimmed as much as possible to prevent an adverse host immune response.

Taken together, these results represent a significant improvement from the current practice of cold storage. Indeed, no dedicated strategy for the storage of living valves exists. Donated valvular allografts most often remain in the donor heart (in traditional static cold storage or in a SherpaPak) until they are prepared for reimplantation in the recipient. Valves are not expected to remain fully viable for more than 3-4 days, and most tissues are allocated for transplantation within 24 hours due to concerns of viability loss. Our findings highlight the potential for longer-term storage of valvular allografts (>1 week). Clinically, we envision the implementation of this storage strategy in situations where a valvular allograft is procured, and a recipient is not immediately available.

A primary design specification of our preservation strategy is to facilitate clinical translation by using only commercially available components. This was inspired by clinically approved preservation solutions such as Custodiol and University of Wisconsin solution. As the components of our preservation solution are all available off-the-shelf, they provide a practical and translationally relevant foundation which can be further developed with clinically approved and/or chemically defined additives. This is in contrast with blood products, which are subject to donor availability, blood-typing, and anticoagulation. Tools such as the Organ Care System may introduce logistic, cost-related, and regulatory hurdles if applied to valvular allografts. Preservation of the entire heart could introduce distribution challenges if the valves are destined to two unique recipients or if the donor myocardium is diseased (often the case for domino donors). Dying myocardium in prolonged normothermic storage may also adversely affect the physiology of the stored valvular allografts.

There are important limitations to this technique. While the leaflet's viability was largely preserved, there were definite changes in the tissue physiology over time. H&E and Movat Pentachrome stains demonstrated shifts to the trilaminar leaflet architecture after 3 weeks ex vivo, highlighting the shortcomings of static normothermic storage. The characteristic layers of the valvular leaflet became less distinguished, as the leaflet became more uniformly collagenous with loss of the glycosaminoglycans and proteoglycans typically residing in the spongiosa layer. Maintenance of the tissue microarchitecture is important, as valve structure is key to its in vivo function, and alterations to the tissue architecture (due to storage conditions or valve disease) are associated with reduced function. Moving forward, it will be critical to evaluate the potential for integrating additional biochemical supplements or mechanical conditioning to improve functional tissue preservation.

We hypothesize that the observed shifts to the tissue microarchitecture may be a result of the loss of physiologic mechanical stimulation. Mechanosensitive resident VICs respond to environmental signals to maintain tissue structure. Mechanical stimulation has been shown to preserve the structure and ECM composition of primary valvular leaflet sections during ex vivo culture, and has been key to the development of physiologic tissue in heart valve tissue engineering.

The start of the ex vivo storage period included a 72-hour period of antibiotic incubation, mimicking the upper limit of the short-term cold storage preceding heart valve transplants clinically. We observed a tendency towards reduced PA viability following just 72 hours of cold storage, in the presence and absence of antibiotics. The rapid loss in PA viability in cold storage may be associated with increased metabolic demand of activated smooth muscle cells, versus quiescent fibroblasts in the valvular leaflet. This is coupled with the increased thickness of the PA (about 3 times that of the leaflet: FIG. 101 and FIG. 107) which requires vascular perfusion for oxygenation. While the requirement for antibiotic incubation introduces a limitation to our study, it provides important insights regarding the individual allograft tissues' viability following ex vivo cold storage, previously explored at shorter timeframes. It will be necessary to repeat these studies using freshly harvested, sterile tissue to eliminate the effects of reduced viability due to shipping- and antibiotic treatment-related cold storage.

Viability of the stored tissues were benchmarked against fresh valves procured within 24 hours prior to receipt, representing metabolically active, living valvular tissue. However, additional controls accounting for the possibility of metabolic reprogramming following exposure to the storage microenvironment are of interest.

Finally, although clinical data support the hypothesis that living valves will grow, the true growth-capacity of ex vivo stored living allogeneic valves has not been evaluated in large animal models (e.g., growing piglet model). The physiologic criteria for “acceptable” donor HVT tissue have yet to be defined clinically. Therefore, a critical ongoing question is which physiologic parameters must be preserved to ensure continued function of the valvular graft. In vivo studies are essential to answering this question given the complexity of the valvular environment and the unpredictability of the graft-host interface. For example, mechanical properties are maintained in cryopreserved valves, vet this does not translate into preserved function in vivo. On the other hand, loss of up to 50% viability in heart valve transplants does not preclude continued growth capacity in piglets. Identifying which parameters determine durability will require controlled studies investigating the effect of unique physiologic elements including microarchitecture, mechanical properties, and viability on long-term durability and function of the implanted graft. This will, in turn, inform acceptable clinical warm and cold ischemic times and guide the continued development of ex vivo preservation strategies.

### CONCLUSIONS

We demonstrate the capacity to preserve valvular allograft tissue viability for up to 7 weeks ex vivo, with maintained metabolic activity and interstitial cell phenotype. This preservation timeline significantly outcompetes the current standard of static cold storage. Microarchitectural shifts were observed throughout this timeframe, and it is anticipated that mechanical conditioning will improve results by promoting conservation of the valvular microstructure and ECM composition. These outcomes represent key preclinical support towards developing a strategy for preserving living allogeneic valves. If validated in vivo, such a strategy may significantly extend the availability of valve sources for transplantation by enabling their off-the-shelf availability. Given the promise of HVT, this may, in turn, significantly positively impact patient outcomes while reducing healthcare costs.

### Aspects

The following Aspects are illustrative only and do not limit the scope of the present disclosure or the appended claims. Any part or parts of any one or more Aspects can be combined with any part or parts of any one or more other Aspects.
Aspect 1. A tissue culture system, comprising: a support configured to be rotated about an axis of rotation; and one or more culture vessels configured to be mounted to at least a portion of the support, a culture vessel comprising: (i) a chamber configured to receive a biological tissue, and (ii) a conduit in communication with the chamber and configured to facilitate a flow of a composition, wherein the support is configured to cause rotation of the one or more culture vessels during rotation of the support, and wherein the conduit is configured to allow for flow of the composition through the chamber during rotation of the culture vessel.
Aspect 2. The tissue culture system of Aspect 1, wherein at least a portion of the support is substantially tubular.
Aspect 3. The tissue culture system of any one of Aspects 1-2, wherein at least a portion of the culture vessel is substantially annular.
Aspect 4. The tissue culture system of any one of Aspects 1-3, wherein the culture vessel is configured to rotate about the axis of rotation of the support during rotation of the support.
Aspect 5. The tissue culture system of any one of Aspects 1-4, wherein at least a portion of the culture vessel is configured to be mounted to the support circumferentially.
Aspect 6. The tissue culture system of any one of Aspects 1-5, wherein the conduit of the culture vessel extends between a first end and a second end, and each of the first end of the conduit and the second end of the conduit is configured to be secured to the chamber of the culture vessel.
Aspect 7. The system of Aspect 6, wherein the conduit of the culture vessel defines a first opening at the first end of the conduit and a second opening at the second end of the conduit, and wherein each of the first opening and the second opening is in fluid communication with the chamber of the culture vessel.
Aspect 8. The tissue culture system of any one of Aspects 6-7, wherein one or more of the first end of the conduit of the culture vessel and the second end of the conduit of the culture vessel includes a first coupling member, wherein the chamber of the culture vessel includes a second coupling member, and wherein the first coupling member of the conduit is configured to be releasably coupled to the second coupling member of the chamber.
Aspect 9. The tissue culture system of any of Aspects 1-8, wherein the chamber of the culture vessel includes a platform configured to secure at least a portion of the biological tissue within the chamber.
Aspect 10. The tissue culture system of any of Aspects 1-9, wherein one or more of the chamber and the conduit of the culture vessel is configured to facilitate unidirectional flow of the composition through the chamber.
Aspect 11. The tissue culture system of any of Aspects 1-10, wherein the biological tissue is an allograft.
Aspect 12. The tissue culture system of Aspect 11, wherein the allograft is a valve of a heart of an animal.
Aspect 13. The tissue culture system of Aspect 11, wherein the allograft is a valve of a human.
Aspect 14. The tissue culture system of any one of Aspects 1-13, wherein the tissue culture system is configured to effect flow of the composition along the biological tissue at a velocity magnitude of within about 30% of a physiological velocity magnitude experienced by the biological tissue in vivo.
Aspect 15. The tissue culture system of any one of Aspects 1-14, wherein the tissue culture system is configured to effect flow of the composition along the biological tissue at a maximum flow velocity magnitude of from about 0.25 m/s to about 1.5 m/s, optionally from about 0.5 m/s to about 1.25 m/s. In some configurations the tissue culture system can be configured to effect flow of the composition along the biological tissue at a flow velocity magnitude of from about 0 m/s to about 2 m/s. The velocity magnitude can vary depending on where a reactor is in its turning cycle, for example, if the reactor is actively turning or restarting the cycle.
Aspect 16. The tissue culture system of any one of Aspects 1-14, wherein the tissue culture system is configured to effect pulsatile flow of the composition along the biological tissue at a rate within about 30% of a physiological flow pulse frequency experienced by the biological tissue in vivo.
Aspect 17. The tissue culture system of Aspect 15, wherein the tissue culture system is configured to effect pulsatile flow of the composition along the biological tissue at a rate of from about 10 to 150 pulses per minute, optionally from about 40 to about 180 pulses per minute.
Aspect 18. The tissue culture system of any one of Aspects 1-17 wherein the tissue culture system is configured to effect flow of the composition along the biological tissue that effects a shear stress on the composition of within about 30% of a physiological shear stress experienced by the biological tissue in vivo.
Aspect 19. The tissue culture system of any one of Aspects 1-18 wherein the tissue culture system is configured to effect flow of the composition along the biological tissue that effects a shear stress on the composition of about 5 dynes/cm² to about 50 dynes/cm², optionally from about 10 dynes/cm² to about 25 dynes/cm².
Aspect 20. The tissue culture system of any one of Aspects 1-19 wherein the tissue culture system is configured to effect flow of the composition along the biological tissue that effects a hydrostatic pressure on the composition of within about 30% of a physiological pressure-which can be a maximum physiological pressure-experienced by the biological tissue in vivo.
Aspect 21. The tissue culture system of any one of Aspects 1-20 wherein the tissue culture system is configured to effect hydrostatic pressure of the composition along the biological tissue that effects a cyclic maximum transvalvular pressure gradient of about 10 mm Hg to about 100 mm Hg, optionally from about 30 mm Hg to about 50 mm Hg.
Aspect 22. The tissue culture system of any of Aspects 1-21, wherein the tissue culture system is configured to perform a rotation schedule that gives rise to flow of the composition across the biological tissue.
Aspect 23. The tissue culture system of any one of Aspects 1-22, further comprising a sensor train configured to determine any one or more of a velocity magnitude of the composition within the tissue culture system, a shear stress within tissue culture system, a pressure within the tissue culture system, and a frequency of a pulsatile flow within the tissue culture system.
Aspect 24. The tissue culture system of any one of Aspects 1-23, further comprising a control train configured to effect any one or more of (1) flow of the composition within about 30% of a physiological velocity magnitude experienced by the biological tissue in vivo, (2) pulsatile flow of the composition along the biological tissue at a rate within about 30% of a physiological flow pulse frequency experienced by the biological tissue in vivo, (3) flow of the composition along the biological tissue that effects a stress on the composition of within about 30% of a physiological shear stress experienced by the biological tissue in vivo, and (4) flow of the composition along the biological tissue that effects a shear on the composition of within about 30% of a physiological shear stress experienced by the biological tissue in vivo.
Aspect 25. A culture vessel configured for culturing a biological tissue, the culture vessel comprising: a chamber configured to receive a biological tissue; and a conduit in communication with the chamber and configured to facilitate a circumferential flow of a composition, wherein at least a portion of the chamber and the conduit are configured to be mounted to a support, and wherein the chamber and the conduit are configured to be rotated about an axis of rotation so as to cause the composition to flow through the chamber.
Aspect 26. The culture vessel of Aspect 25, wherein at least a portion of the culture vessel is substantially annular.
Aspect 27. The culture vessel of any of Aspects 25-26, wherein the conduit of the culture vessel extends between a first end and a second end, and wherein each of the first end of the conduit and the second end of the conduit is secured to the chamber of the culture vessel.
Aspect 28. The culture vessel of Aspect 25, wherein the conduit of the culture vessel defines a first opening at the first end of the conduit and a second opening at the second end of the conduit, and wherein each of the first opening and the second opening are in fluid communication with the chamber of the culture vessel.
Aspect 29. The culture vessel of any one of Aspects 25-28, wherein one or more of the chamber and the conduit of the culture vessel is configured to facilitate unidirectional flow of the composition through the chamber.
Aspect 30. The culture vessel of any one of Aspects 25-29, further comprising a unidirectional valve configured to permit passage of the composition in a single direction.
Aspect 31. The culture vessel of any one of Aspects 25-30, wherein the chamber comprises at least one member configured to engage with the biological tissue.
Aspect 32. The culture vessel of Aspect 31, wherein the member comprises a projection.
Aspect 33. The culture vessel of any one of Aspects 25-32, wherein the culture vessel comprises at least one channel configured to direct composition communicated from an interior portion of the biological tissue to an exterior portion of the biological tissue.
Aspect 34. The culture vessel of Aspect 33, wherein the at least one channel is characterized as a backflow channel.
Aspect 35. The culture vessel of any one of Aspects 33-34, wherein the biological tissue is a heart valve, and wherein the at least one channel is configured to direct composition communicated from an interior portion of the heart valve to an exterior portion of the heart valve.
Aspect 36. The culture vessel of any one of Aspects 25-34, wherein the culture vessel comprises composition disposed therein. Such a composition can be a composition according to the present disclosure, for example a composition according to any one of Aspects 45-52.
Aspect 37. A method for culturing biological tissue, the method comprising: rotating an annular culture vessel having a biological tissue retained therein so as to give rise to motion of a fluid composition within the annular culture vessel, the rotating giving rise to flow of the fluid composition along the biological tissue.
Aspect 38. The method of Aspect 37, wherein the flow of the fluid composition recapitulates fluid flow conditions experienced by the biological tissue in vivo.
Aspect 39. The method of Aspect 38, wherein the flow of the fluid composition recapitulates any one or more of (1) flow of the composition within about 30% of a physiological velocity magnitude experienced by the biological tissue in vivo, (2) pulsatile flow of the composition along the biological tissue at a rate within about 30% of a physiological flow pulse frequency experienced by the biological tissue in vivo, (3) flow of the composition along the biological tissue that effects a stress on the composition of within about 30% of a physiological shear stress experienced by the biological tissue in vivo, and (4) flow of the composition along the biological tissue that effects a shear on the composition of within about 30% of a physiological shear stress experienced by the biological tissue in vivo.
Aspect 40. The method of any of Aspects 37-39, wherein the biological tissue is secured within a chamber of the annular culture vessel.
Aspect 41. The method of any one of Aspects 37-40, wherein the rotation is in accordance with a predetermined rotation schedule.
Aspect 42. The method of any one of Aspects 37-41, wherein the rotation is bidirectional and/or oscillatory.
Aspect 43. The method of any one of Aspects 37-42, wherein the biological tissue is heart tissue.
Aspect 44. The method of any one of Aspects 37-42, wherein the rotation gives rise to from 40 to 180 pulses per minute of fluid composition flowing along the biological tissue.
Aspect 45. A composition for tissue preservation, comprising: a base solvent: at least one of an animal serum and an animal-free serum: an antioxidant: insulin, an insulin analog, or an insulin mimetic: a glutamine: at least one of an antibiotic and an antimycotic; and optionally at least one of albumin and ascorbic acid.

In one embodiment, the composition comprises Advanced DMRM/F12, KOSR, GlutaMAX™, human albumin, ascorbic acid, insulin, and antibiotic/antimycotic.

In an exemplary embodiment, the base solvent is a medium, such as Dulbecco's Modified Eagle Medium. In some embodiments, additives and supplements are also used including, Glucose, L-Glutamine, Phenol red, Sodium pyruvate, HEPES, Bovine serum albumin, Fetal bovine serum, KnockOut serum (THERMOFISHER™), CDM-HD (KD Bio), Panexin CD (Ilex Life Sciences), FastGro Synthetic (MP Biomedicals), or an alternative chemically-defined replacement to fetal bovine serum, and dextran. In some embodiments, basal antibiotics are used, including penicillin, streptomycin, vancomycin, imipenem, amphotericin, gentamicin, cefotaxime, fluconazole, polymyxin, lincomycin.

In another embodiment, the base solvent is Advanced DMEM/F12 with Non-Essential Amino Acids and Sodium Pyruvate. In some embodiments, additives and supplement are used including Knock Out Serum, ascorbic acid, albumin, insulin, L-glutamine (GLUTAMAX), and an antibiotic and antimycotic solution.

The basal preservation solution can include additional supplements, such as signaling factors, hormones, steroids, small molecules: cell-derived materials, anti-apoptotic molecules or compounds: immuno-modulatory molecules or compounds: metabolic aids, and antioxidants; and phenotypic cues. In some embodiments, signaling factors, hormones, steroids, small molecules include fibroblast growth factor 2, vascular endothelial growth factor, bone morphogenic protein 2, retinoic acid. TGFB inhibitors (i.e., SB-431542. SB 525334, SB 505124, dorsomorphin, LY 364947, LY3200882), bone morphogenic protein 1 inhibitors (i.e., K02288, LDN212854), Y-27632, SIRT1 activator 3, muscone, nitric oxide, dexamethasone, hydrocortisone, and/or other corticosteroids, ascorbic acid, L-ascorbic acid 2-phosphate, insulin, thyroid hormone (T3) or other physiological hormones, and caffeine. In some embodiments, cell-derived materials include iPS-derived extracellular vesicles: placenta-derived extracellular vesicles: valve endothelial cell-derived extracellular vesicles: valve interstitial cell-derived extracellular vesicles: cardiomyocyte-derived extracellular vesicles: microRNAs. In some embodiments, immuno-modulatory molecules or compounds include IL-4: IL-6: IL-10; IL-11: IL-13: IL-1 antagonist: bilirubin; carbon monoxide; and mesenchymal stem cell conditioned media. In some embodiments anti-apoptotic molecules or compounds include Necrostatin-1: broad-spectrum caspase inhibitor: individual caspase inhibitors; and autophagy inhibitors. In some embodiments, metabolic aids include ATP, arachidonic acid (AA). supplemental oxygen, and supplemental carbon dioxide. In some embodiments, antioxidants include glutathione. In some embodiments, phenotypic cues include SB431542 (SB). One can provide oxygen supplementation to the composition, for example via one or more of synthetic blood, red blood cells, oxygen carriers, and exogenous oxygen delivery. Additional additives can also include dextran and Albumax™ or other lipid-rich BSA.
Aspect 46. The composition of Aspect 45, wherein the animal-free serum comprises a knock-out serum replacement.
Aspect 47. The composition of any one of Aspects 45-46, further comprising a supplement, the supplement optionally comprising: glucose, L-glucose, dextran, phenol red, sodium pyruvate, and HEPES.
Aspect 48. The composition of Aspect 57, wherein the supplement comprises any one or more of a cell-derived molecule, a cell-derived compound, an immunomodulatory molecule, an immunomodulatory compound, an anti-apoptotic molecule, an anti-apoptotic compound, a metabolic acid molecule, and a metabolic acid compound.
Aspect 49. The composition of any one of Aspects 45-48, wherein the antibiotic comprises anyone or more of penicillin, streptomycin, vancomycin, imipenem, amphotericin, gentamicin, cefotaxime, fluconazole, polymyxin, and lincomycin.
Aspect 50. The composition of any one of Aspects 45-49, further comprising any one or more of a signaling molecule and a signaling compound.
Aspect 51. The composition of any one of Aspects 45-50, wherein the composition comprises any one or more of (1) from about 1 to 10 vol % of the at least one of an animal serum and an animal-free serum, (2) from about 0.1 to about 10 mg/mL ascorbic acid, (3) from about 0 to 1 mg/mL albumin, (4) from about 1 to about 100 U/L insulin, (5) from about 1 to about 5 vol % of at least one of L-alanyl-L-glutamine dipeptide and/or L-glutamine, and (6) from about 1 to about 5 vol % of the at least one of an antibiotic and an antimycotic.

Insulin can be present, for example, at from about 1 U/L to about 100 U/L, from about 1 U/L to about 50 U/L, from about 1 U/L to about 35 U/L, from about 1 U/L to about 25 U/L, or from about 1 U/L to about 10 U/L, as non-limiting, example concentrations. It should be understood that all intermediate ranges and sub-ranges from about 1 U/L to about 100 U/L are within the scope of the present disclosure. An insulin analog or an insulin mimetic can be present at the foregoing concentrations provided in the context of insulin: an insulin analog or insulin mimetic can also be present at a concentration that provides an equivalent effect as the foregoing concentrations provided in the context of insulin.
Aspect 52. The composition of any one of Aspects 45-51, wherein the composition comprises from about 0.01 to about 250 μg/mL albumin.

As an example, a composition can include a base solvent: at least one of an animal serum and an animal-free serum: ascorbic acid, L-ascorbic acid 2-phosphate, and/or one or more other antioxidants: insulin and/or one or more insulin-mimetic trace metals, including, for example zinc: at least one of L-alanyl-L-glutamine dipeptide and L-glutamine: at least one of an antibiotic and an antimycotic agent; and optionally albumin. It should be understood that ascorbic acid, L-ascorbic acid 2-phosphate are example antioxidants, and that other antioxidants can be used. L-glutamine and L-alanyl-L-glutamine dipeptide are considered suitable, non-limiting glutamines. As described, an animal-free serum can comprise a knock-out serum replacement.

Also as described, a composition can include a supplement: a supplement can comprise any one or more of glucose, L-glucose, dextran, phenol red, sodium pyruvate, and HEPES. A supplement can also include any one or more of a cell-derived molecule, a cell-derived compound, an immunomodulatory molecule, an immunomodulatory compound, an anti-apoptotic molecule, an anti-apoptotic compound, a metabolic aid molecule, and a metabolic aid compound: a supplement can also include a signaling factor, a hormone, a steroid, a small molecule, and/or a phenotypic cue.

Signaling factors, hormones, steroids, and/or small molecules that can be included in the disclosed compositions include fibroblast growth factor 2, vascular endothelial growth factor, bone morphogenic protein 2, retinoic acid, TGFB inhibitors (i.e., SB-431542, SB 525334, SB 505124, dorsomorphin, LY 364947, LY3200882), bone morphogenic protein 1 inhibitors (i.e., K02288, LDN212854), Y-27632, SIRT1 activator 3, muscone, nitric oxide, dexamethasone, hydrocortisone, ascorbic acid, insulin, thyroid hormone (T3), and caffeine.

Cell-derived materials that can be present in the disclosed compositions include include iPS-derived extracellular vesicles: placenta-derived extracellular vesicles: valve endothelial cell-derived extracellular vesicles; valve interstitial cell-derived extracellular vesicles: cardiomyocyte-derived extracellular vesicles: microRNAs.

Immuno-modulatory molecules or compounds that can be present in the disclosed compositions include IL-4; IL-6; IL-10; IL-11: IL-13: IL-1 antagonist: bilirubin; carbon monoxide; and mesenchymal stem cell conditioned media.

Anti-apoptotic molecules or compounds that can be present in the disclosed compositions include Necrostatin-1: broad-spectrum caspase inhibitor; individual caspase inhibitors; and autophagy inhibitors.

Metabolic aids that can be present in the disclosed compositions include ATP, arachidonic acid (AA), supplemental oxygen (in the form of synthetic blood, oxygen carriers, or direct oxygen supplementation), and supplemental carbon dioxide. In some embodiments, antioxidants include glutathione. In some embodiments, phenotypic cues include SB431542 (SB).
Aspect 53. A method, comprising storing a tissue in a composition according to any one of Aspects 45-52.
Aspect 54. The method of Aspect 53, wherein the tissue comprises an allograft.
Aspect 55. The method of Aspect 54, wherein the tissue comprises heart valve tissue.
Aspect 56. The method of any one of Aspects 53-55, wherein the storing is normothermic.
Aspect 57. The method of any one of Aspects 53-56, wherein the storing is for up to about 3 weeks.
Aspect 58. The method of any one of Aspects 53-56, wherein the storing is for up to about 7 weeks.
Aspect 59. The method of any one of Aspects 53-58, wherein after storing for about 4 weeks, the tissue exhibits a change of less than about 10% in at least one of AlamarBlue cell viability, glucose consumption, lactate release, TUNEL testing, thickness, and cellularity.

What is claimed:

1. A tissue culture system, comprising:
a support configured to be rotated about an axis of rotation; and
one or more culture vessels configured to be mounted to at least a portion of the support, a culture vessel comprising: (i) a chamber configured to receive a biological tissue, and (ii) a conduit in communication with the chamber and configured to facilitate a flow of a composition,
wherein the support is configured to cause rotation of the one or more culture vessels during rotation of the support, and
wherein the conduit is configured to allow for flow of the composition through the chamber during rotation of the culture vessel.

2. The tissue culture system of claim 1, wherein (a) at least a portion of the support is substantially tubular, (b) wherein at least a portion of the culture vessel is substantially annular, or both (a) and (b).

3. The tissue culture system of claim 1, wherein the culture vessel is configured to rotate about the axis of rotation of the support during rotation of the support.

4. The tissue culture system of claim 1, wherein the conduit of the culture vessel extends between a first end and a second end, and each of the first end of the conduit and the second end of the conduit is configured to be secured to the chamber of the culture vessel.

5. The tissue culture system of claim 1, wherein one or more of the chamber and the conduit of the culture vessel is configured to facilitate unidirectional flow of the composition through the chamber.

6. The tissue culture system of claim 1, wherein (a) the tissue culture system is configured to effect flow of the composition along the biological tissue at a velocity magnitude of within about 30% of a physiological velocity magnitude experienced by the biological tissue in vivo, (b) the tissue culture system is configured to effect flow of the composition along the biological tissue at a maximum velocity magnitude of from about 0.25 to about 1.5 m/s, optionally from about 0.5 to about 1.25 m/s, or both (a) and (b).

7. The tissue culture system of claim 1, wherein (a) the tissue culture system is configured to effect pulsatile flow of the composition along the biological tissue at a rate within about 30% of a physiological flow pulse frequency experienced by the biological tissue in vivo, (b) the tissue culture system is configured to effect pulsatile flow of the composition along the biological tissue at a rate of from about 10 to 180 pulses per minute, optionally from about 60 to about 120 pulses per minute, or both (a) and (b).

8. The tissue culture system of claim 1, wherein (a) the tissue culture system is configured to effect flow of the composition along the biological tissue that effects a stress on the composition of within about 30% of a physiological shear stress experienced by the biological tissue in vivo, (b) the tissue culture system is configured to effect flow of the composition along the biological tissue that effects a shear on the composition of about 5 dynes/cm² to about 50 dynes/cm², optionally from about 10 dynes/cm² to about 25 dynes/cm², or both (a) and (b).

9. The tissue culture system of claim 1, wherein (a) the tissue culture system is configured to effect flow of the composition along the biological tissue that effects a hydrostatic pressure on the composition of within about 30% of the physiological maximum pressure experienced by the biological valve tissue in vivo, (b) the tissue culture system is configured to effect hydrostatic pressure of the composition along the biological tissue that effects a pressure on the composition of about 10 mm Hg to about 100 mm Hg, optionally from about 30 mm Hg to about 50 mm Hg, or both (a) and (b).

10. The tissue culture system of claim 1, wherein the tissue culture system is configured to perform a rotation schedule that gives rise to flow of the composition across the biological tissue.

11. The tissue culture system of claim 1, further comprising (a) a sensor train configured to determine any one or more of a velocity magnitude of the composition within the tissue culture system, a shear stress within tissue culture system, a pressure within the tissue culture system, and a frequency of a pulsatile flow within the tissue culture system, (b) a control train configured to effect any one or more of (1) flow of the composition within about 30% of a physiological velocity magnitude experienced by the biological tissue in vivo, (2) pulsatile flow of the composition along the biological tissue at a rate within about 30% of a physiological flow pulse frequency experienced by the biological tissue in vivo, (3) flow of the composition along the biological tissue that effects a stress on the composition of within about 30% of a physiological shear stress experienced by the biological tissue in vivo, and (4) flow of the composition along the biological tissue that effects a shear on the composition of within about 30% of a physiological shear stress experienced by the biological tissue in vivo, or both (a) and (b).

12. A culture vessel configured for culturing a biological tissue, the culture vessel comprising:
a chamber configured to receive a biological tissue; and
a conduit in communication with the chamber and configured to facilitate a circumferential flow of a composition,
wherein at least a portion of the chamber and the conduit are configured to be mounted to a support, and
wherein the chamber and the conduit are configured to be rotated about an axis of rotation so as to cause the composition to flow through the chamber,
optionally wherein at least a portion of the culture vessel is substantially annular.

13. The culture vessel of claim 12, wherein (a) the conduit of the culture vessel extends between a first end and a second end, and wherein each of the first end of the conduit and the second end of the conduit is secured to the chamber of the culture vessel, (b) the conduit of the culture vessel defines a first opening at the first end of the conduit and a second opening at the second end of the conduit, and wherein each of the first opening and the second opening are in fluid communication with the chamber of the culture vessel, or both (a) and (b).

14. The culture vessel of claim 12, wherein one or more of the chamber and the conduit of the culture vessel is configured to facilitate unidirectional flow of the composition through the chamber, the culture vessel optionally comprising a unidirectional valve configured to permit passage of the composition in a single direction, the chamber optionally comprising at least one member configured to engage with the biological tissue, the member optionally comprising a projection.

15. The culture vessel of claim 12, wherein the culture vessel comprises at least one channel configured to direct composition communicated from an interior portion of the biological tissue to an exterior portion of the biological tissue, the at least one channel optionally characterized as a backflow channel.

16. A method for culturing biological tissue, the method comprising:
rotating an annular culture vessel having a biological tissue retained therein so as to give rise to motion of a fluid composition within the annular culture vessel,
the biological tissue optionally secured within a chamber of the annular culture vessel; and
the rotating giving rise to flow of the fluid composition along the biological tissue,
the flow of the fluid composition optionally recapitulating fluid flow conditions experienced by the biological tissue in vivo.

17. The method of claim 16, wherein the flow of the fluid composition recapitulates any one or more of (1) flow of the composition within about 30% of a physiological velocity magnitude experienced by the biological tissue in vivo, (2) pulsatile flow of the composition along the biological tissue at a rate within about 30% of a physiological flow pulse frequency experienced by the biological tissue in vivo, (3) flow of the composition along the biological tissue that effects a stress on the composition of within about 30% of a physiological shear stress experienced by the biological tissue in vivo, and (4) flow of the composition along the biological tissue that effects a shear on the composition of within about 30% of a physiological shear stress experienced by the biological tissue in vivo.

18. The method of claim 16, wherein the composition comprises any one or more of (1) from about 1 to 10 vol % of the at least one of an animal serum and an animal-free serum, (2) from about 0.1 to about 10 mg/mL ascorbic acid, (3) from about 0 to 1 mg/mL albumin, (4) from about 1 to about 100 U/L insulin, (5) from about 1 to about 5 vol % of the at least one of L-alanyl-L-glutamine dipeptide and L-glutamine, and (6) from about 1 to about 5 vol % of the at least one of an antibiotic and an antimycotic.

19. A method, comprising: storing a tissue in a composition according to claim 18, the tissue optionally comprising an allograft, the storing optionally being normothermic.

20. A composition for tissue preservation, comprising:
a base solvent;
at least one of an animal serum and an animal-free serum;
an antioxidant;
insulin, an insulin analog, or an insulin mimetic;
a glutamine;
at least one of an antibiotic and an antimycotic; and
optionally at least one of albumin and ascorbic acid.
